(19) United States
(12) Patent Application Publication
(10) Pub. No.: US 2013/0224151 A1
(43) Pub. Date: Aug. 29, 2013

(54) Use of FAAH Inhibitors for Treating Abdominal, Visceral and Pelvic Pain

(71) Applicants: James Philip Pearson, Cambridge, MA (US); Mark G. Currie, Sterling, MA (US); Yvette Tache, Los Angeles, CA (US); Muriel Larauche, Los Angeles, CA (US)

(72) Inventors: James Philip Pearson, Cambridge, MA (US); Mark G. Currie, Sterling, MA (US); Yvette Tache, Los Angeles, CA (US); Muriel Larauche, Los Angeles, CA (US)

(73) Assignees: UNITED STATES OF AMERICA, Washington, DC (US); IRONWOOD PHARMACEUTICALS, INC, Cambridge, MA (US)

(21) Appl. No.: 13/637,761

(22) PCT Filed: Mar. 31, 2011
(86) PCT No.: PCT/US11/30835

§ 371 Date: May 15, 2013

Related U.S. Application Data
(60) Provisional application No. 61/319,493, filed on Mar. 31, 2010.

(51) Int. Cl. A61K 31/404; A61K 45/06; A61K 31/4439; A61K 31/325; C07D 209/22; C07D 401/12

(52) U.S. Cl. CPC A61K 31/404; C07D 209/22; C07D 401/12; A61K 31/4439; A61K 31/325; A61K 45/06
USPC 424/85.6; 514/419; 514/339; 514/484; 514/327; 514/161; 514/226.5; 514/411; 514/243; 514/20.5; 514/171; 424/133.1; 514/11.7

(57) ABSTRACT

The present disclosure relates to methods of using fatty acid amide hydrolase (FAAH) inhibitors alone or in combination for the treatment or prevention of abdominal, visceral or pelvic pain. Also described herein are pharmaceutical compositions comprising a FAAH inhibitor, alone or in combination with an additional therapeutic agent for the treatment of abdominal, visceral or pelvic pain.

### PRIORITY CLAIM

This application claims priority to U.S. Provisional Application Ser. No. 61/319,493, filed on Mar. 31, 2010. The entire contents of the aforementioned application are herein incorporated by reference.

### GOVERNMENT SUPPORT

This invention was created in the performance of a Cooperative Research and Development Agreement with the Department of Veterans Affairs, an agency of the U.S. Government, which has certain rights in this invention.

### TECHNICAL FIELD

The present disclosure relates to methods of using fatty acid amide hydrolase (FAAH) inhibitors and pharmaceutically acceptable salts thereof, alone or in combination with one or more additional therapeutic agents, for the treatment or prevention of visceral, abdominal and pelvic pain associated with various diseases. The disclosure is also directed to pharmaceutical compositions comprising FAAH inhibitors for use in the treatment and or prevention of visceral, abdominal and pelvic pain associated with those diseases.

### BACKGROUND

Pain is a major ailment affecting the general population and the most common reason for physician consultations in the US. Chronic pain affects an estimated 86 million American adults to some degree and it is estimated that primary and secondary expenditures associated with pain average about $100 billion annually in the United States alone. Pain is a major symptom in many medical conditions, and can significantly interfere with a person's quality of life and general functioning.

Abdominal, visceral or pelvic pain may be caused by a number of diseases and can be chronic or acute in nature. In addition, abdominal, visceral and pelvic pain may affect various body systems (e.g. gastrointestinal, liver, pancreas, urological, gynecological, etc).

Irritable bowel syndrome (IBS, including all its variants, such as IBS-d, IBS-c and IBS-a), is the most common cause of recurrent, intermittent abdominal pain and it affects up to 20% of the population. Other types of abdominal, visceral or pelvic pain may be associated with inflammation (such as in inflammatory bowel disease, pelvic inflammatory disease, pancreatitis), trauma, cancer (such as a result of obstructions caused by colorectal cancer), hernias, vascular disease (such as in occlusive intestinal ischemia), exaggerated pain sensitivity (e.g., bladder, bowel, prostate or uterine pain), and gynecological conditions (such as in dysmenorrhea or endometriosis).

Additional compounds and pharmaceutical compositions for the treatment and/or prevention of abdominal, visceral and/or pelvic pain are therefore highly desirable.

### SUMMARY

In one aspect, the invention provides a method of treating or preventing abdominal pain, visceral pain or pelvic pain in a patient in need thereof, comprising administering a therapeutically or prophylactically effective amount of a FAAH inhibitor, alone or in combination with a therapeutically or prophylactically effective amount of one or more additional therapeutic agents to said patient. It also provides a method for the use of a FAAH inhibitor, or a pharmaceutically acceptable salt thereof, for the manufacture of a medicament for the treatment or prevention of abdominal pain, visceral pain or pelvic pain.

In another aspect, the invention provides pharmaceutical compositions comprising a FAAH inhibitor, alone or in combination with one or more additional therapeutic agents, for use in the treatment of abdominal, visceral or pelvic pain. In another aspect, the invention provides a pharmaceutical composition comprising a Mu opioid receptor agonist, a 5HT3 antagonist, an anti-diarrheal compound, a bile acid sequestrant, a mast cell stabilizer, or any combination of these therapeutic agents thereof, in combination with a FAAH inhibitor, for the treatment or prevention of abdominal pain, visceral pain or irritable bowel syndrome.

In a further aspect, the invention provides a kit comprising at least two separate unit dosage forms (A) and (B), wherein (A) is a therapeutic agent, a combination of more than one therapeutic agent, a pharmaceutically acceptable salt thereof, or a pharmaceutical composition thereof, and (B) is a FAAH inhibitor, pharmaceutically acceptable salt thereof, or a pharmaceutical composition thereof.

### BRIEF DESCRIPTION OF THE DRAWINGS

FIG. 1 shows the effects of the FAAH inhibitor compound A, the FAAH inhibitor compound B and vehicle control on basal sensitivity in the colorectal distension model.

FIG. 2 shows the effects of the FAAH inhibitor compound A and a vehicle control on stress-induced visceral hypersensitivity in rats.

FIG. 3 shows the effects of the FAAH inhibitor compound B and a vehicle control on stress-induced visceral hypersensitivity in rats.

FIGS. 4A, 4B, 4C and 4D show the effects of the FAAH inhibitor URB597, compound A and compound B pre-treatments versus vehicle on cortagine-induced visceral hypersensitivity to colorectal distension (CRD) in rats.

FIG. 4E shows the effects on colorectal distension (CRD) of the FAAH inhibitor URB597 pre-treatments compared with vehicle pre-treatments on cortagine-induced visceral hypersensitive or vehicle-treated control rats.

FIGS. 5A, 5B and 5C show the endocannabinoids N-arachidonoyl-ethanolamide (AEA, anandamide), N-oleoyl-ethanolamide (OEA), and N-palmitoyl-ethanolamide (PEA) levels in the brain (FIG. 5A), jejunum (FIG. 5B) and ascending colon (FIG. 5C) after a single administration of the FAAH inhibitor URB597 in cortagine-induced visceral hypersensitive rats.

FIGS. 5D-5F shows the endocannabinoids N-arachidonoyl-ethanolamide (AEA, anandamide), N-oleoyl-ethanolamide (OEA), and N-palmitoyl-ethanolamide (PEA) levels in the brain (FIG. 5D), jejunum (FIG. 5E) and ascending colon (FIG. 5F) after a single administration of the FAAH inhibitor URB597 in vehicle-treated or cortagine-induced visceral hypersensitive rats.

FIGS. 6A and 6B show the effects of the FAAH inhibitor URB 597 and a vehicle control on basal sensitivity in the colorectal distension model.

FIGS. 7A and 7B show the effects of the FAAH inhibitor URB 597 and a vehicle controls on stress-induced visceral hypersensitivity in rats.

FIGS. 8A-8J provides references and structures for exemplary known FAAH inhibitors.

The figures are provided by way of examples and are not intended to limit the scope of the present invention.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls. The compounds described herein may be defined by their chemical structures and/or chemical names. Where a compound is referred to by both a chemical structure and a chemical name, and the chemical structure and chemical name conflict, the chemical structure is determinative of the compound's identity.

The term “halo” or “halogen” refers to any radical of fluorine, chlorine, bromine or iodine.

As used herein, the term “cyano” refers to —CN or —C≡N.

The term “hydroxyl” or “hydroxy” refers to —OH.

The term “alkyl” refers to a hydrocarbon chain that may be a straight chain or branched chain, containing the indicated number of carbon atoms. For example, C₁-C₁₂ alkyl indicates that the group may have from 1 to 12 (both inclusive) carbon atoms in it (i.e., 1, 2, 3, 4, 5, 6, 7, 8, 9, 10, 11 or 12). The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Examples of “arylalkyl” or “aralkyl” include, but are not limited to, benzyl and 9-fluorenyl groups.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “alkoxy” refers to an —O-(alkyl) radical. Thus, for example, alkoxy or alkoxyl can refer to groups of 1, 2, 3, 4, 5, 6, 7 or 8 carbon atoms of a straight, branched, cyclic configuration and combinations thereof attached to the parent structure through an oxygen atom. Examples include, but are not limited to, methoxy, ethoxy, propoxy, isopropoxy, cyclopropyloxy, cyclohexyloxy and the like. Lower-alkoxy refers to groups containing one to four carbons.

The term “cycloalkyl” as employed herein includes saturated monocyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to cyclopropyl, cyclobutyl, cyclopentyl, norbornyl, cyclohexyl and adamantyl.

The term “carbocycle” as employed herein includes saturated, partially unsaturated or unsaturated monocyclic, bicyclic, tricyclic, or polycyclic hydrocarbon groups having 3 to 12 carbons, wherein any ring atom capable of substitution can be substituted by a substituent. Carbocycles can be aromatic, e.g., a phenyl ring is an example of a carbocycle. A subset of the carbocycles is the non-aromatic carbocycles.

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Example rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of —OR^{o} as in Formula D₁:

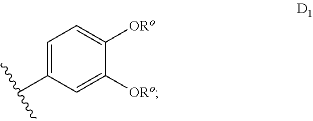

the two occurrences of —OR^{o}, wherein R^{o} is, for example Me, are taken together with the carbon atoms to which they are bound to form a fused 6-membered oxygen containing ring as in Formula D₂:

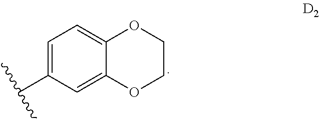 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

The term “substituents” refers to a group “substituted” on an alkyl, cycloalkyl, alkenyl, alkynyl, heterocyclyl, heterocycloalkenyl, cycloalkenyl, aryl, or heteroaryl group or other group at any atom of the group. The group can be singly or multiply substituted and where multiply substituted, the substituents are independent. Suitable substituents include, without limitation: F, Cl, Br, I, alkyl, alkenyl, alkynyl, alkoxy, acyloxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)n alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents. In some cases the substituents are selected from: F, Cl, Br and I. In other cases the substituents are selected from: halogen, optionally independently halogen substituted C₁-C₃ alkyl, optionally independently halogen substituted C₁-C₃ alkoxy, hydroxy, cyano, nitro and amino. In some cases the substituents are selected from aryl groups. In some cases the substituents are selected from heteroaryl groups. In some cases the substituents are selected from: halogen, hydroxy, and C₁-C₃ alkyl. In some cases, the substituents are selected from: halogen, hydroxy, and C₁-C₃ alkyl and C₁-C₃ alkoxyl.

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces the use of isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Example isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ³²P, ³³P, ³⁵S, ¹⁸F, ³⁶Cl, ¹²³I and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures known to those having ordinary skill in the art, by substituting an isotopically labeled reagent for a non-isotopically labeled reagent.

### Embodiments

In one aspect, the present invention provides a method for the treatment or prevention of pain, such as for example, abdominal, visceral and pelvic pain, in a patient in need thereof, comprising administering a therapeutically or prophylactically effective amount of a FAAH inhibitor to said patient.

In some embodiments, the pain is visceral pain. In other embodiments, the pain is abdominal pain. In still other embodiments, the pain is pelvic pain. In some embodiments, the pain is selected from:
(a) gastrointestinal pain: stomach pain, rectal pain, bowel pain, intestinal pain, intestinal cramps, pain and/or discomfort associated with irritable bowel syndrome, pain and/or discomfort associated with inflammatory bowel disease; pain and/or discomfort associated with functional dyspepsia, pain and/or discomfort associated with functional abdominal pain, pain and/or discomfort associated with ulcerative colitis, Crohn's disease or celiac disease; chest pain associated with gastro-esophageal reflux disease;
(b) pancreas pain, liver pain; cardiac pain;
(c) urological, renal or gynecological pain: kidney pain, ureter pain, bladder pain, prostate pain, gynecological pain, ovarian pain, uterine pain, labor pain, vulvar pain, vaginal pain, dysmenorrhea, dyspareuinia, endometriosis, menstrual cramps, post-menopausal pelvic pain, pain and/or discomfort associated with vulvodynia, pain and/or discomfort associated with interstitial cystitis or painful bladder syndrome, pain and/or discomfort associated with prostatitis, pain associated with inflammatory pelvic disease.

In other embodiments, the pain is gastrointestinal pain and is selected from: stomach pain, rectal pain, bowel pain, intestinal pain, intestinal cramps, pain and/or discomfort associated with irritable bowel syndrome (IBS), pain and/or discomfort associated with inflammatory bowel disease (IBD), pain and/or discomfort associated with functional dyspepsia, pain and/or discomfort associated with functional abdominal pain, pain and/or discomfort associated with ulcerative colitis, Crohn's disease or celiac disease; chest pain associated with gastro-esophageal reflux disease.

In still other embodiments, the pain is selected from: pain and/or discomfort associated with irritable bowel syndrome or pain and/or discomfort associated with inflammatory bowel disease. In further embodiments, the pain and/or discomfort is associated with diarrhea-predominant IBS (IBS-d), constipation-predominant IBS (IBS-c) or alternating IBS (IBS-a). In yet further embodiments, the pain and/or discomfort is associated with IBS-d. In other embodiments, the pain and/or discomfort is associated with ulcerative colitis, Crohn's disease or celiac disease.

In some embodiments, the pain is urological, renal or gynecological pain and is selected from: kidney pain, ureter pain, bladder pain, prostate pain, gynecological pain, ovarian pain, uterine pain, labor pain, vulvar pain, vaginal pain, dysmenorrhea, dyspareuinia, endometriosis, menstrual cramps, post-menopausal pelvic pain, pain and/or discomfort associated with vulvodynia, pain and/or discomfort associated with interstitial cystitis or painful bladder syndrome, pain and/or discomfort associated with prostatitis, pain associated with inflammatory pelvic disease.

In other embodiments, the pain is abdominal, visceral or pelvic pain caused by cancer, by bacterial infections, viral infections, parasitic infections, surgery, trauma, medications, and exposure to noxious chemicals or digestive disorders. In still other embodiments, the pain is abdominal discomfort, soft-tissue pain, caused by pancreatitis, gallstones, diverticulitis, kidney stones, gastritis or referred pain.

In some embodiments, the patient is a human.

In another aspect, the present invention provides a method for the treatment or prevention of abdominal, visceral or pelvic pain, in a patient in need thereof, comprising administering a therapeutically or prophylactically effective amount of a FAAH inhibitor or a pharmaceutically acceptable salt thereof, in combination with at least one other therapeutic agent or pharmaceutically acceptable salt thereof, to said patient.

In some embodiments, the additional therapeutic agent or agents are selected from: a painkiller, a Mu opioid receptor agonist, a non-steroidal anti-inflammatory drug (NSAID), a pain relieving agent, an opiate receptor agonists, a cannabinoid receptor agonist, an anti-infective agent, a sodium channel blocker, an N-type calcium channel blocker, a local anesthetic, a VR1 agonist, an anti-inflammatory and/or immunosuppressive agent, an antidepressant, an anti-emetic agent, a corticosteroid, a proton pump inhibitor, a leukotriene antagonist, a nicotinic acetylcholine receptor agonist, a P2X3 receptor antagonist, a NGF agonist and antagonist, an NK1 and NK2 antagonist, a NMDA antagonist, a GABA modulator, an anti-cancer agent, an anti hyperlipidemia drug, an appetite suppressing agent, an anti-diabetic medication, a serotonergic and noradrenergic modulator, a GI agent, a GCC (Guanylate Cyclase C) agonist, a 5HT4 agonist, a 5HT3 antagonist, a bile acid sequestrant, a mast cell stabilizer or an anti-diarrheal compound.

In other embodiments:
(a) said painkiller is acetaminophen or paracetamol;
(b) said Mu opioid receptor agonist is loperamide;
(c) said non-steroidal anti-inflammatory drug is selected from: propionic acid derivatives (e.g., alminoprofen, benoxaprofen, bucloxic acid, carprofen, fenhufen, fenoprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (meclofenamic acid, mefe-namic acid, and tolfenamic acid), biphenyl-carboxylic acid derivatives, oxicams (isoxicam, meloxicam, piroxicam, sudoxicam and tenoxican), salicylates (acetyl salicylic acid, sulfasalazine), pyrazolones (apazone, bezpiperylon, feprazone, mofebutazone, oxyphenbutazone, phenylbutazone), or a COX-2 inhibitor, such as, for example, a COX-2 inhibitor in the coxibs family (celecoxib, deracoxib, valdecoxib, rofecoxib, parecoxib, nimesulide, etoricoxib);
(d) said other pain relieving agent is gabapentin, topical capsaicin, tanezumab, esreboxetine or pregabalin;
(e) said opiate receptor agonist is morphine, propoxyphene (Darvon™), tramadol, hydrocodone, oxycodoneor buprenorphin;
(f) said cannabinoid receptor agonist is Dronabinol™, A9-THC, CP-55940, WIN-55212-2, HU-210, cannabis, marijuana, marijuana extract, levonatradol, nabilone, ajulemic acid, Cannador® or Sativex™;
(g) said sodium channel blocker is carbamazepine, mexiletine, lamotrigine, lidocaine, tectin, NW-1029 or CGX-1002;
(h) said N-type calcium channel blocker is ziconotide, NMED-160, SPI-860; serotonergic and noradrenergic modulators such as SR-57746, paroxetine, duloxetine, clonidine, amitriptyline or citalopram; anticonvulsants such as gabapentin and pregalabin;
(i) said VR1 agonist and antagonist is NGX-4010, WL-1002, ALGRX-4975, WL-10001 or AMG-517;
(j) said anti-inflammatory and/or immunosuppressive agent is methotrexate, cyclosporin A (including, for example, cyclosporin microemulsion), tacrolimus, corticosteroids, statins, interferon beta, Remicade (Infliximab™), Enbrel (Etanercept™) or Humira (Adalimumab™);
(k) said antidepressant is an SSRIs (e.g., fluoxetine, citalopram, femoxetine, fluvoxamine, paroxetine, indalpine, sertraline, zimeldine), a combined SSRI and 5HT1A partial agonist (e.g., vilazodone), a tricyclic antidepressant (e.g., imipramine, amitriptiline, chlomipramine and nortriptiline), a therapeutic antidepressant (e.g., bupropion and amineptine) or an SNRIs (e.g., duloxetine, venlafaxine and reboxetine);
(l) said 5HT3 antagonist is ondansetron (Zofran™), granisetronmetoclopramide, ramosetron (Irribow™) or alosetron (Lotronex™);
(m) said corticosteroid is betamethasone, budesonide, cortisone, dexamethasone, hydrocortisone, methylprednisolone, prednisolone, prednisone or triamcinolone;
(n) said proton pump inhibitor is omeprazole, lansoprazole, rabeprazole, esomeprazole or pantroprazole;
(o) said leukotriene antagonist is zafirlukast, montelukast, pranlukast;
(oo) said 5-lipoxygenase inhibitors is zileuton or PF-04191834;
(p) said nicotinic acetylcholine receptor agonist is ABT-202, A-366833, ABT-594; BTG-102, A-85380 or CGX1204;
(q) said P2X3 receptor antagonist is A-317491, ISIS-13920 or AZD-9056;
(r) said NGF agonist and antagonist is tanezumab, RI-724, RI-1024, AMG-819, AMG-403 or PPH 207;
(s) said NK1 and NK2 antagonist is DA-5018, R-116301; CP-728663 or ZD-2249;
(t) said NMDA antagonist is NER-MD-11, CNS-5161, EAA-090, AZ-756, CNP-3381; potassium channel modulators is CL-888, ICA-69673 or retigabine;
(u) said GABA modulator is lacosamide or propofol;
(v) said anti-cancer agent is tyrosine kinase inhibitors imatinib (Gleevec/Glivec™) or gefitinib (Iressa™), fluorouracil, 5-FU (Adrucil™), bevacizumab (Avastin™), irinotecan (Camptosar™), oxaliplatin (Eloxatin™), cetuximab (Erbitux™), panitumumab (Vectibix™), leucovorin (Wellcovorin™) or capecitabine (Xeloda™);
(w) said anti hyperlipidemia drug is a statin, ezetimibe or niacin;
(x) said appetite suppressing agent is sibutramine, taranabant or rimonabant;
(y) said anti-diabetic medication is insulin, tolbutamide (Orinase™), acetohexamide (Dymelor™), tolazamide (Tolinase™), chlorpropamide (Diabinese™), glipizide (Glucotrol™), glyburide (Diabeta™, Micronase™, Glynase™), glimepiride (Amaryl™), gliclazide (DiamicronT), repaglinide (Prandin™), nateglinide (Starlix™), pramlintide (Symlin™) or exanatide (Byetla™);
(z) said serotonergic or noradrenergic modulator is SR-57746, paroxetine, duloxetine, clonidine, amitriptyline, citalopram, or flibanserin;
(aa) said GI agent is a laxative (e.g. lubiprostone (Amitiza™), Fybogel®, Regulan®, Normacol® and the like), a gastrointestinal agent used for the treatment of idiopathic chronic constipation and constipation-predominant IBS, a GI motility stimulant (e.g. domperidone, metoclopramide, mosapride, itopride) or an antispasmodic drug (e.g. anticholinergics, hyoscyamine or dicyclomine);
(bb) said GCC (Guanylate Cyclase C) agonists is linaclotide;
(cc) said 5HT4 agonist is tegasarod;
(dd) said bile acid sequestrant is questran, cholesevelan, sevelamer, cholestipol or cholestyramine;
(ee) said mast cell stabilizer is cromolyn or nedocromil; and
(ff) said anti-diarrhea compound is octreotide, an antiperistaltic agent (e.g. loperamide (Imodium™, Pepto Diarrhea™)), tamoxifen, a bulking agent, an anti-estrogen (e.g. droloxifene, TAT-59 orraloxifene), tormentil root extract (Potejntilla tormentilla) from the family Rosaceae, bismuth subsalicylate (e.g. Pepto-Bismol™), diphenoxylate, diphenoxylate with atropine (Lomotil™, Lomocot™), oat bran, psyllium, calcium carbonate or an astringent (e.g., tannins).

In another aspect, the present invention provides a kit comprising at least two separate unit dosage forms (A) and (B), wherein (A) is a therapeutic agent, a combination of two or more therapeutic agents, a pharmaceutically acceptable salt thereof, or a pharmaceutical composition thereof, and (B) is a FAAH inhibitor, pharmaceutically acceptable salt thereof, or a pharmaceutical composition thereof.

In another aspect, the present invention provides for the use of a FAAH inhibitor or a pharmaceutically acceptable salt thereof for the manufacture of a medicament for the treatment or prevention of abdominal, visceral or pelvic pain.

In another aspect, the invention provides pharmaceutical compositions comprising a FAAH inhibitor or a pharmaceutically acceptable salt thereof, alone or in combination with one or more therapeutic agents or pharmaceutically acceptable salts thereof, for use in the treatment of abdominal, visceral or pelvic pain. In some embodiments, said pharmaceutical composition comprises a painkiller, a Mu opioid receptor agonist, an anti-diarrheal compound, a 5HT3 antagonist or a bile acid sequestrant in combination with a FAAH inhibitor or a pharmaceutically acceptable salt thereof, for the treatment or prevention of visceral pain, abdominal pain or IBS. In other embodiments said pharmaceutical composition comprises loperamide in combination with a FAAH inhibitor or pharmaceutically acceptable salt thereof for the treatment or prevention of visceral pain, abdominal pain or IBS. In other embodiments the pharmaceutical composition comprises ramosetron, alosetron or ondansetron in combination with a FAAH inhibitor or pharmaceutically acceptable salt thereof for the treatment or prevention of irritable bowel syndrome (IBS), visceral pain or abdominal pain. In still other embodiments the pharmaceutical composition comprises a compound used for the treatment of stool consistency, or the urgency or frequency of bowel movements in combination with a FAAH inhibitor or pharmaceutically acceptable salt thereof for the treatment or prevention of irritable bowel syndrome, visceral pain or abdominal pain. In further embodiments the pharmaceutical composition comprises questran in combination with a FAAH inhibitor or pharmaceutically acceptable salt thereof for the treatment or prevention of irritable bowel syndrome, visceral pain or abdominal pain. In yet other embodiments the pharmaceutical composition comprises cromolyn or nedocromil in combination with a FAAH inhibitor or pharmaceutically acceptable salt thereof for the treatment or prevention of IBS, visceral pain or abdominal pain.

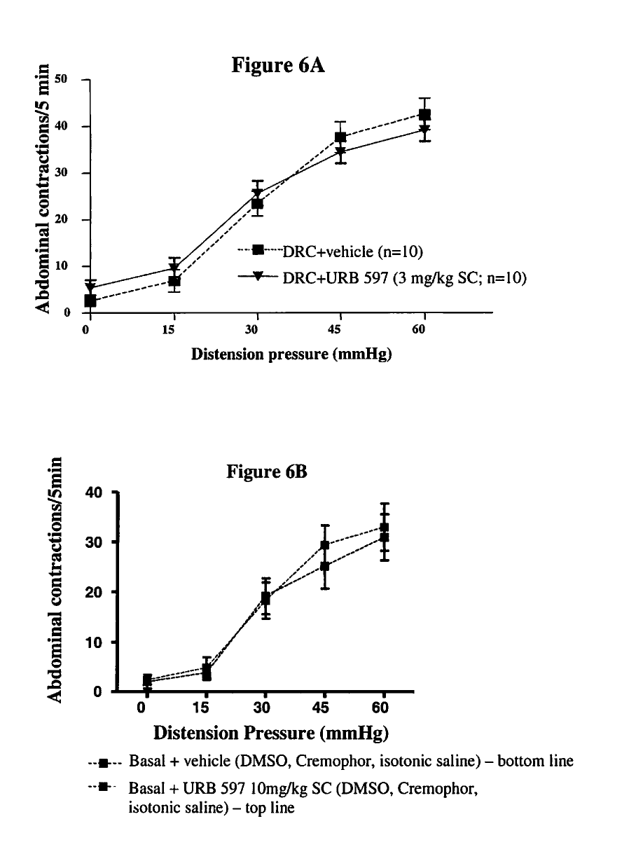

In some embodiments of the above methods, pharmaceutical compositions, kits and uses, the FAAH inhibitor is selected from those provided in FIG. 6.

In some embodiments of the above methods, pharmaceutical compositions, kits and uses, the FAAH inhibitor is SA-47, SA-72, BMS-1, Org-23295, OL-135, OL-92, URB-597, URB-532, URB-694, URB-524, LY2183240, OL-135, OMDM-119, OMDM-122, OMDM-132, α-KH-7, AA-5-HT, CAY-10401, PF-750, PF-3845, PF-622, BMS-469908, SSR-411298, TK-25, PF-04457845, JNJ-245, JNJ-28833155, JNJ-1661010, AM-374, URB-880, JP83, JP104, compound 210 from EP 2065369, compounds 1, 4 or 5 from WO2008/047229, compounds 18, 19, 21, 26, 52 or 59 from WO 2006/074025, compound 229 from WO 2009/151991, compound 129 from WO 2009/152025, compound 3 from WO2010/017079, example #5 from WO2010/101274 or compounds 1-11 from S. Pillarisetti et al., “Pain and beyond: fatty acid amides and fatty acid amide hydrolase inhibitors in cardiovascular and metabolic diseases”, Drug Discov. Today (2009), doi:10.1016/j.drudis.2009.08.002.

In some embodiments of the above methods, pharmaceutical compositions, kits and uses, the FAAH inhibitor is a compound disclosed in WO2010/141817, WO2010/141809, WO2010/135360, WO2010/130945, WO2010/130944, WO2010/130943, WO2010/124113, WO2010/117014, WO2010/118159, WO2010/118155, WO2010/089510, WO2010/074588, WO2010/074587, WO2010/0068453, WO2010/0068452, WO2010/064597, WO2010/058318, WO2010/059610, WO2010/055267, WO2010/053120, WO2010/049841, WO2010/039186, WO2010/017079, WO2010/010288, WO2010/007966, WO2010/005572, WO2010/101274, WO2009/154785, WO2009/109504, WO2009/084970, WO 2009/151991, WO 2009/152025, WO 2009/127943, WO 2009/127944, WO 2009/127946, WO 2009/127949, WO 2009/127948, WO 2009/126691, WO 2009/109743, WO 2009/105220, US 2009/0163508, EP 2065369, WO2008/157740, US 2009/0118503, US 2009/0111778, WO 2009/051666, US 2009/0030074, WO 2009/011904, WO2008/150492, WO2008/145839, WO2008/147553, US2010/41651, WO2008/745843, US2010/41670, WO2008/129129, US2009/099240, WO 2008/047229, WO 2008/153752, US 2008/0312226, WO2008/020866, WO 2008/022976, WO 2008/100977, WO2008/030752, WO2008/042892, WO2008/030532, US 2008/0045513, WO2008/021625, US2008/089845, US2008/119549, WO2007/098142, WO2007/020888, WO2007/070892, US2009/48263, WO2006/117461, US2008/103197, WO2006/044617, WO 2006/054652, WO 2006/074025, WO2006/117461, US2007/027141, US 2006/0173184, WO 2003/065989, WO 2004/033422, WO2004/033652, WO2004/053066, WO2004/099176, US2006/89344, WO 2006/088075, EP1923388, WO 2008/063300, WO 2005/090322, US 2009/0143365, WO 2007/140005, WO2007/005510, US2007/0004741, WO 2006/0258700 or WO 2007/061862.

In some embodiments of the above methods, pharmaceutical compositions, kits and uses, the FAAH inhibitor is a compound of Formula I:

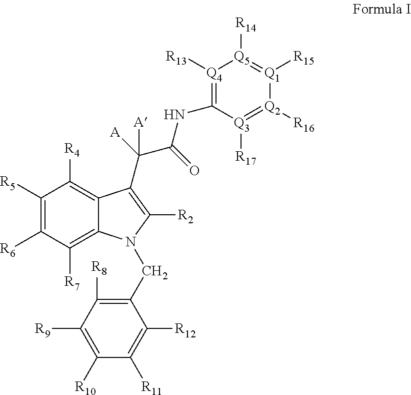

wherein:
each of Q1, Q2, Q3, Q4, and QS is independently N or C;
A and A′ are independently: hydroxyl or an optionally independently substituted C₁ to C₃ alkoxy or A and A′ taken together are ═O, ═N(OH) or ═NOCH₃ or A and A′ together with the carbon to which they are attached form a cyclic ketal containing a total of 4 or 5 carbon atoms which can be optionally independently substituted;
R₂ is halogen, hydroxyl, —NO₂, an optionally independently substituted C₁-C₅ alkyl, an optionally independently substituted C₁-C₅ alkoxy, an optionally independently substituted C₂-C₅ alkenyl, an optionally independently substituted C₂-C₅ alkynyl, —CN, —C(O)OH, an optionally independently substituted cyclopropyl, —C(O)NR₂ₐR_{2b}, or —NR₂ₐR_{2b}, wherein R₂ₐ and R_{2b} are independently H or C1-C3 alkyl;
each of R₄, R₅, R₆ and R₇ is independently: H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b} or —NRₐR_{b}; wherein Rₐ and R_{b} are independently H, an optionally independently substituted C₁-C₆ alkyl or an optionally independently substituted C₃-C₆ cycloalkyl;
each of R₈, R₉, R₁₀, R₁₁ and R₁₂ is independently: H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b} or —NRₐR_{b}; wherein Rₐ and R_{b} are independently H, an optionally independently substituted C₁-C₆ alkyl, or an optionally independently substituted C₃-C₆ cycloalkyl;
when Q₅ is C, R₁₄ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b} or —NRₐR_{b}; wherein Rₐ and R_{b} are independently H, an optionally independently substituted C₁-C₆ alkyl, or an optionally independently substituted C₃-C₆ cycloalkyl;
when Q₅ is N, R₁₄ is missing;
when Q₂ is C, R₁₆ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b} or —NRₐR_{b}; wherein Rₐ and R_{b} are independently H, optionally independently substituted C₁-C₆ alkyl, or an optionally independently substituted C₃-C₆ cycloalkyl;
when Q₂ is N, R₁₆ is missing;
when Q₁ is C, R₁₅ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b} or —NRₐR_{b}; wherein Rₐ and R_{b} are independently H, optionally independently substituted C₁-C₆ alkyl, or an optionally independently substituted C₃-C₆ cycloalkyl;
when Q₁ is N, R₁₅ is missing;
when Q₄ is C, R₁₃ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b} or —NRₐR_{b}; wherein Rₐ and R_{b} are independently H, optionally independently substituted C₁-C₆ alkyl, or an optionally independently substituted C₃-C₆ cycloalkyl;
when Q₄ is N, R₁₃ is missing;
when Q₃ is C, R₁₇ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b} or —NRₐR_{b}; wherein Rₐ and R_{b} are independently H, optionally independently substituted C₁-C₆ alkyl, or an optionally independently substituted C₃-C₆ cycloalkyl;
and
when Q₃ is N, R₁₇ is missing.

In some embodiments, when Q1, Q2, Q3, Q4, and Q5 are C; R2 is methyl; and A and A′ taken together are ═O, then (1) R₁₅ is not C(O)NH₂ and R₁₀ is not Cl; (2) R₈, R₉, R₁₀, R₁₁, and R₁₂ are not all H and R₁₃ and R₁₇ are not both methyl; and (3) R₈, R₉, R₁₀, R₁₁, R₁₂, R₁₃, R₁₄, R₁₅, R₁₆, R₁₇ are not all H, in said compound of Formula I or pharmaceutically acceptable salts thereof.

In further embodiments, the FAAH inhibitor is a compound of Formula A-2, Formula A-3 or Formula A-4, or a pharmaceutically acceptable salt thereof:

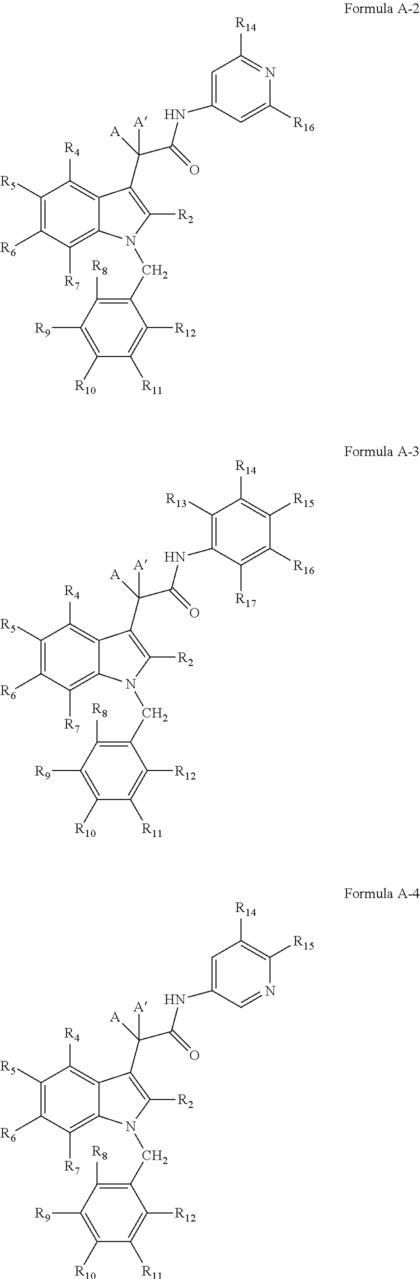

In further embodiments, the FAAH inhibitor is a compound of Formula A-5 or Formula A-7, or a pharmaceutically acceptable salt thereof:

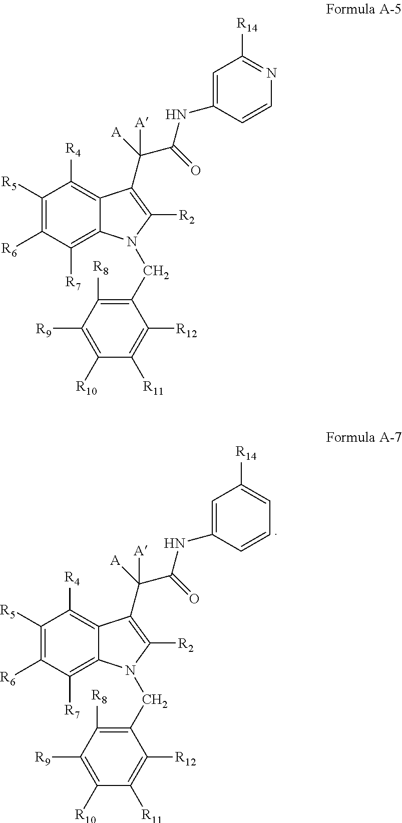

In some embodiments, A and A′ taken together are ═O in said compounds of formulae I and A-2 to A-7. In other embodiments, R₂ is an optionally independently halogen substituted C₁-C₃ alkyl or cyclopropyl in said compounds. In still further embodiments, R₂ is methyl in said compounds. In still other embodiments, one or two of R₈, R₉, R₁₀, R₁₁ and R₁₂ are halogen and the rest are H in said compounds. In further embodiments, R₁₀ is Cl or F and R₈, R₉, R₁₁ and R₁₂ are H. In yet further embodiments, R₄ and R₇ are H in said compound. In yet further embodiments, R₆ is H in said compounds. In yet further embodiments, R₅ is selected from: ethoxy, methoxy, ethyl, methyl, halogen and H in said compounds. In still further embodiments, R₅ is methoxy or methyl.

In yet further embodiments, each of R₁₃, R₁₅, R₁₆ and R₁₇ is independently selected from H, a halogen, —NO₂, —CN, —C(O)OH, hydroxyl, a C₁-C₅ alkyl, a C₂-C₅ alkenyl, a C₂-C₅ alkynyl, a C₁-C₅ alkoxy, —C(O)NRₐR_{b} or —NRₐR_{b}; wherein Rₐ and R_{b} are independently H, a C₁-C₆ alkyl, or a C₃-C₆ cycloalkyl in said compounds of Formula I and A-2 to A-4. In yet other embodiments, R₁₄ is halogen or an optionally independently substituted methoxy and both R₁₃ and R₁₇ are H in said compounds.

In still other embodiments, R₁₄ is halogen or an optionally independently substituted methoxy in said compounds of Formulae I and A-2 to A-7. In still further embodiments, R₁₄ is Cl, F or —OCH₃ in said compounds.

In some embodiments of the above methods, pharmaceutical compositions, kits and uses, the FAAH inhibitor is selected from the following, or a pharmaceutically acceptable salt thereof:
2-[1-(4-chlorobenzyl)-2-methyl-1H-indol-3-yl]-N-(2-chloropyridin-4-yl)-2-oxoacetamide
2-[1-(4-chlorobenzyl)-2-methyl-1H-indol-3-yl]-N-(3-methoxyphenyl)-2-oxoacetamide
2-[1-(4-chlorobenzyl)-5-methoxy-2-methyl-1H-indol-3-yl]-2-oxo-N-pyridin-2-ylacetamide
2-[2-chloro-1-(4-chlorobenzyl)-5-methoxy-1H-indol-3-yl]-2-oxo-N-pyridin-3-ylacetamide
2-[1-(4-chlorobenzyl)-5-methoxy-2-methyl-1H-indol-3-yl]-2-oxo-N-pyridin-4-ylacetamide
2-[1-(4-chlorobenzyl)-2,5-dimethyl-1H-indol-3-yl]-2-oxo-N-pyridin-4-ylacetamide
2-[1-(4-chlorobenzyl)-5-methoxy-2-methyl-1H-indol-3-yl]-2-oxo-N-phenylacetamide
2-[2-chloro-1-(4-chlorobenzyl)-5-methoxy-1H-indol-3-yl]-2-oxo-N-pyridin-4-ylacetamide
2-[2-chloro-1-(4-chlorobenzyl)-5-methoxy-1H-indol-3-yl]-2-oxo-N-pyrimidin-4-ylacetamide
2-[2-chloro-1-(4-chlorobenzyl)-5-methoxy-1H-indol-3-yl]-N-(2-chloropyridin-4-yl)-2-oxoacetamide
2-[1-(4-chlorobenzyl)-5-methoxy-2-methyl-1H-indol-3-yl]-N-(2-chloropyridin-4-yl)-2-oxoacetamide
2-[1-(4-chlorobenzyl)-5-ethoxy-2-methyl-1H-indol-3-yl]-2-oxo-N-phenylacetamide
2-[1-(4-chlorobenzyl)-5-methoxy-2-methyl-1H-indol-3-yl]-N-(3-methoxyphenyl)-2-oxoacetamide
2-[1-(4-chlorobenzyl)-2,5-dimethyl-1H-indol-3-yl]-2-oxo-N-phenylacetamide
2-[1-(2,4-dichlorobenzyl)-5-methoxy-2-methyl-1H-indol-3-yl]-2-oxo-N-pyridin-4-ylacetamide
2-[1-(4-chlorobenzyl)-5-methoxy-2-methyl-1H-indol-3-yl]-N-(3-chlorophenyl)-2-oxoacetamide
2-[1-(4-chlorobenzyl)-5-methoxy-2-methyl-1H-indol-3-yl]-2-oxo-N-pyrimidin-4-ylacetamide
2-[1-(4-chlorobenzyl)-2,5-dimethyl-1H-indol-3-yl]-2-oxo-N-pyridin-3-ylacetamide
2-[1-(4-chlorobenzyl)-5-methoxy-2-methyl-1H-indol-3-yl]-2-oxo-N-pyridin-3-ylacetamide
2-[1-(2,4-dichlorobenzyl)-5-methoxy-2-methyl-1H-indol-3-yl]-2-oxo-N-pyridin-3-ylacetamide
2-[1-(4-chlorobenzyl)-5-methoxy-2-methyl-1H-indol-3-yl]-N-(4-chlorophenyl)-2-oxoacetamide
2-[1-(4-chlorobenzyl)-5-methoxy-2-methyl-1H-indol-3-yl]-N-(4-methoxyphenyl)-2-oxoacetamide
2-[5-chloro-1-(4-chlorobenzyl)-2-methyl-1H-indol-3-yl]-2-oxo-N-pyridin-2-ylacetamide
2-[1-(4-chlorobenzyl)-2-isopropyl-5-methoxy-1H-indol-3-yl]-2-oxo-N-pyridin-4-ylacetamide
2-[1-(4-chlorobenzyl)-5-methoxy-2-methyl-1H-indol-3-yl]-N-(2-chlorophenyl)-2-oxoacetamide
2-[1-(4-chlorobenzyl)-2-isopropyl-5-methoxy-1H-indol-3-yl]-2-oxo-N-pyridin-3-ylacetamide
2-[1-(4-chlorobenzyl)-2-isopropyl-5-methoxy-1H-indol-3-yl]-2-oxo-N-phenylacetamide
2-[1-(4-chlorobenzyl)-5-methoxy-2-methyl-1H-indol-3-yl]-N-(2-methoxyphenyl)-2-oxoacetamide
2-[1-(4-chlorobenzyl)-5-ethoxy-2-methyl-1H-indol-3-yl]-2-oxo-N-pyridin-4-ylacetamide
2-[1-(4-chlorobenzyl)-2-methyl-1H-indol-3-yl]-2-oxo-N-pyridin-4-ylacetamide
2-[1-(4-chlorobenzyl)-5-hydroxy-2-methyl-1H-indol-3-yl]-2-oxo-N-phenylacetamide
2-[1-(4-chlorobenzyl)-2-methyl-1H-indol-3-yl]-2-oxo-N-pyridin-3-ylacetamide
2-[1-(4-chlorobenzyl)-2-methyl-1H-indol-3-yl]-2-oxo-N-phenylacetamide
N-(3-chlorophenyl)-2-[1-(2,4-dichlorobenzyl)-5-methoxy-2-methyl-1H-indol-3-yl]-2-oxoacetamide
2-[1-(2,4-dichlorobenzyl)-5-methoxy-2-methyl-1H-indol-3-yl]-N-(3-methoxyphenyl)-2-oxoacetamide
2-[1-(2,4-dichlorobenzyl)-5-methoxy-2-methyl-1H-indol-3-yl]-N-(5-methoxy-2-methylphenyl)-2-oxoacetamide
2-[1-(2,4-dichlorobenzyl)-5-methoxy-2-methyl-1H-indol-3-yl]-2-oxo-N-pyrimidin-4-ylacetamide
2-[1-(2,4-dichlorobenzyl)-5-methoxy-2-methyl-1H-indol-3-yl]-2-oxo-N-phenylacetamide
2-[1-(4-chlorobenzyl)-5-methoxy-2-methyl-1H-indol-3-yl]-N-(3-hydroxypyridin-2-yl)-2-oxoacetamide
2-[1-(4-chlorobenzyl)-2-methyl-1H-indol-3-yl]-2-oxo-N-pyrimidin-4-ylacetamide
2-[1-(4-chlorobenzyl)-2-methyl-1H-indol-3-yl]-N-(3-chlorophenyl)-2-oxoacetamide
2-[1-(4-chlorobenzyl)-5-hydroxy-2-methyl-1H-indol-3-yl]-2-oxo-N-pyridin-4-ylacetamide
2-[1-(4-chlorobenzyl)-5-methoxy-2-methyl-1H-indol-3-yl]-N-(2-fluorophenyl)-2-oxoacetamide
2-[1-(4-chlorobenzyl)-5-methoxy-2-methyl-1H-indol-3-yl]-N-(3,5-dichlorophenyl)-2-oxoacetamide
2-[1-(4-chlorobenzyl)-5-methoxy-2-methyl-1H-indol-3-yl]-N-(3-fluorophenyl)-2-oxoacetamide.
2-[1-(4-chlorobenzyl)-5-methoxy-2-methyl-1H-indol-3-yl]-N-(4-fluorophenyl)-2-oxoacetamide
2-[1-(4-chlorobenzyl)-5-methoxy-2-methyl-1H-indol-3-yl]-N-(6-methoxypyrimidin-4-yl)-2-oxoacetamide
2-[2-chloro-1-(4-chlorobenzyl)-5-methoxy-1H-indol-3-yl]-N-(3-chlorophenyl)-2-oxoacetamide
2-[2-chloro-1-(4-chlorobenzyl)-5-methoxy-1H-indol-3-yl]-N-(3-methoxyphenyl)-2-oxoacetamide
2-[5-chloro-1-(4-chlorobenzyl)-2-methyl-1H-indol-3-yl]-2-oxo-N-pyridin-4-ylacetamide
2-[5-chloro-1-(4-chlorobenzyl)-2-methyl-1H-indol-3-yl]-2-oxo-N-pyrimidin-4-ylacetamide
2-[5-chloro-1-(4-chlorobenzyl)-2-methyl-1H-indol-3-yl]-N-(2-chloropyridin-4-yl)-2-oxoacetamide
2-[5-chloro-1-(4-chlorobenzyl)-2-methyl-1H-indol-3-yl]-N-(3-chlorophenyl)-2-oxoacetamide
2-[5-chloro-1-(4-chlorobenzyl)-2-methyl-1H-indol-3-yl]-N-(3-methoxyphenyl)-2-oxoacetamide
2-(1-benzyl-2,5-dimethyl-1H-indol-3-yl)-N-(2-methoxypyridin-4-yl)-2-oxoacetamide
2-(1-benzyl-2-methyl-1H-indol-3-yl)-N-(2-methoxypyridin-4-yl)-2-oxoacetamide
2-(1-benzyl-5-methoxy-2-methyl-1H-indol-3-yl)-N-(2-methoxypyridin-4-yl)-2-oxoacetamide
2-[1-(2,4-dichlorobenzyl)-2,5-dimethyl-1H-indol-3-yl]-N-(2-methoxypyridin-4-yl)-2-oxoacetamide
2-[1-(2,4-dichlorobenzyl)-2,5-dimethyl-1H-indol-3-yl]-N-(3-fluorophenyl)-2-oxoacetamide
2-[1-(2,4-dichlorobenzyl)-2-methyl-1H-indol-3-yl]-N-(2-methoxypyridin-4-yl)-2-oxoacetamide
2-[1-(2,4-dichlorobenzyl)-5-fluoro-2-methyl-1H-indol-3-yl]-N-(2-methoxypyridin-4-yl)-2-oxoacetamide
2-[1-(2,4-dichlorobenzyl)-5-methoxy-2-methyl-1H-indol-3-yl]-N-(2-methoxypyridin-4-yl)-2-oxoacetamide
2-[1-(2,4-difluorobenzyl)-2,5-dimethyl-1H-indol-3-yl]-N-(2-methoxypyridin-4-yl)-2-oxoacetamide
2-[1-(2,4-difluorobenzyl)-2-methyl-1H-indol-3-yl]-N-(2-methoxypyridin-4-yl)-2-oxoacetamide
2-[1-(2,4-difluorobenzyl)-5-methoxy-2-methyl-1H-indol-3-yl]-N-(2-methoxypyridin-4-yl)-2-oxoacetamide
2-[1-(2-chloro-4-fluorobenzyl)-2,5-dimethyl-1H-indol-3-yl]-N-(2-methoxypyridin-4-yl)-2-oxoacetamide
2-[1-(2-chloro-4-fluorobenzyl)-2-methyl-1H-indol-3-yl]-N-(2-methoxypyridin-4-yl)-2-oxoacetamide
2-[1-(2-chloro-4-fluorobenzyl)-5-methoxy-2-methyl-1H-indol-3-yl]-N-(2-methoxypyridin-4-yl)-2-oxoacetamide
2-[1-(2-chlorobenzyl)-5-methoxy-2-methyl-1H-indol-3-yl]-N-(2-methoxypyridin-4-yl)-2-oxoacetamide
2-[1-(3-chlorobenzyl)-5-methoxy-2-methyl-1H-indol-3-yl]-N-(2-methoxypyridin-4-yl)-2-oxoacetamide
2-[1-(4-chloro-2-fluorobenzyl)-2,5-dimethyl-1H-indol-3-yl]-N-(2-methoxypyridin-4-yl)-2-oxoacetamide
2-[1-(4-chloro-2-fluorobenzyl)-2-methyl-1H-indol-3-yl]-N-(2-methoxypyridin-4-yl)-2-oxoacetamide
2-[1-(4-chloro-2-fluorobenzyl)-5-methoxy-2-methyl-1H-indol-3-yl]-N-(2-methoxypyridin-4-yl)-2-oxoacetamide
2-[1-(4-chlorobenzyl)-2,5-dimethyl-1H-indol-3-yl]-2-oxo-N-pyrimidin-4-ylacetamide
2-[1-(4-chlorobenzyl)-2,5-dimethyl-1H-indol-3-yl]-N-(2-chloropyridin-4-yl)-2-oxoacetamide
2-[1-(4-chlorobenzyl)-2,5-dimethyl-1H-indol-3-yl]-N-(2-methoxypyridin-4-yl)-2-oxoacetamide
2-[1-(4-chlorobenzyl)-2,5-dimethyl-1H-indol-3-yl]-N-(3-chlorophenyl)-2-oxoacetamide
2-[1-(4-chlorobenzyl)-2,5-dimethyl-1H-indol-3-yl]-N-(3-methoxyphenyl)-2-oxoacetamide
2-[1-(4-chlorobenzyl)-2-methyl-1H-indol-3-yl]-N-(2-methoxypyridin-4-yl)-2-oxoacetamide
2-[1-(4-chlorobenzyl)-5-ethoxy-2-methyl-1H-indol-3-yl]-N-(2-chloropyridin-4-yl)-2-oxoacetamide
2-[1-(4-chlorobenzyl)-5-ethoxy-2-methyl-1H-indol-3-yl]-N-(2-methoxypyridin-4-yl)-2-oxoacetamide
2-[1-(4-chlorobenzyl)-5-ethoxy-2-methyl-1H-indol-3-yl]-N-(3-methoxyphenyl)-2-oxoacetamide
2-[1-(4-chlorobenzyl)-5-fluoro-2-methyl-1H-indol-3-yl]-2-oxo-N-pyridin-4-ylacetamide
2-[1-(4-chlorobenzyl)-5-fluoro-2-methyl-1H-indol-3-yl]-2-oxo-N-pyrimidin-4-ylacetamide
2-[1-(4-chlorobenzyl)-5-fluoro-2-methyl-1H-indol-3-yl]-N-(2-chloropyridin-4-yl)-2-oxoacetamide
2-[1-(4-chlorobenzyl)-5-fluoro-2-methyl-1H-indol-3-yl]-N-(2-methoxypyridin-4-yl)-2-oxoacetamide
2-[1-(4-chlorobenzyl)-5-fluoro-2-methyl-1H-indol-3-yl]-N-(3-chlorophenyl)-2-oxoacetamide
2-[1-(4-chlorobenzyl)-5-fluoro-2-methyl-1H-indol-3-yl]-N-(3-methoxyphenyl)-2-oxoacetamide
2-[1-(4-chlorobenzyl)-5-methoxy-2-methyl-1H-indol-3-yl]-2-oxo-N-[3-(trifluoromethoxy)phenyl]acetamide
2-[1-(4-chlorobenzyl)-5-methoxy-2-methyl-1H-indol-3-yl]-2-oxo-N-[3-(trifluoromethyl)phenyl]acetamide
2-[1-(4-chlorobenzyl)-5-methoxy-2-methyl-1H-indol-3-yl]-N-(2,6-difluorophenyl)-2-oxoacetamide
2-[1-(4-chlorobenzyl)-5-methoxy-2-methyl-1H-indol-3-yl]-N-(2-ethoxypyridin-4-yl)-2-oxoacetamide
2-[1-(4-chlorobenzyl)-5-methoxy-2-methyl-1H-indol-3-yl]-N-(2-fluoropyridin-4-yl)-2-oxoacetamide
2-[1-(4-chlorobenzyl)-5-methoxy-2-methyl-1H-indol-3-yl]-N-(2-methoxypyridin-4-yl)-2-oxoacetamide
2-[1-(4-chlorobenzyl)-5-methoxy-2-methyl-1H-indol-3-yl]-N-(3-chloro-4-fluorophenyl)-2-oxoacetamide
2-[1-(4-chlorobenzyl)-5-methoxy-2-methyl-1H-indol-3-yl]-N-(3-ethoxyphenyl)-2-oxoacetamide
2-[1-(4-chlorobenzyl)-5-methoxy-2-methyl-1H-indol-3-yl]-N-(3-ethylphenyl)-2-oxoacetamide
2-[1-(4-chlorobenzyl)-5-methoxy-2-methyl-1H-indol-3-yl]-N-(3-fluoropyridin-4-yl)-2-oxoacetamide
2-[1-(4-chlorobenzyl)-5-methoxy-2-methyl-1H-indol-3-yl]-N-(3-methylphenyl)-2-oxoacetamide
2-[1-(4-chlorobenzyl)-5-methoxy-2-methyl-1H-indol-3-yl]-N-(4-methoxypyridin-2-yl)-2-oxoacetamide
2-[1-(4-chlorobenzyl)-5-methoxy-2-methyl-1H-indol-3-yl]-N-(5-methoxypyridin-2-yl)-2-oxoacetamide
2-[1-(4-chlorobenzyl)-5-methoxy-2-methyl-1H-indol-3-yl]-N-(6-ethoxypyridin-3-yl)-2-oxoacetamide
2-[1-(4-chlorobenzyl)-5-methoxy-2-methyl-1H-indol-3-yl]-N-(6-methoxypyridin-2-yl)-2-oxoacetamide
2-[1-(4-chlorobenzyl)-5-methoxy-2-methyl-1H-indol-3-y]-N-(6-methoxypyridin-3-yl)-2-oxoacetamide
2-[1-(4-fluorobenzyl)-2,5-dimethyl-1H-indol-3-yl]-N-(2-methoxypyridin-4-yl)-2-oxoacetamide
2-[1-(4-fluorobenzyl)-2-methyl-1H-indol-3-yl]-N-(2-methoxypyridin-4-yl)-2-oxoacetamide
2-[1-(4-fluorobenzyl)-5-methoxy-2-methyl-1H-indol-3-yl]-N-(2-methoxypyridin-4-yl)-2-oxoacetamide
2-[1-(4-fluorobenzyl)-5-methoxy-2-methyl-1H-indol-3-yl]-N-(3-fluorophenyl)-2-oxoacetamide
2-[2-chloro-1-(4-chlorobenzyl)-5-methoxy-1H-indol-3-yl]-N-(2-methoxypyridin-4-yl)-2-oxoacetamide
2-[2-chloro-1-(4-chlorobenzyl)-5-methyl-1H-indol-3-yl]-2-oxo-N-pyridin-4-ylacetamide
2-[2-chloro-1-(4-chlorobenzyl)-5-methyl-1H-indol-3-yl]-2-oxo-N-pyrimidin-4-ylacetamide
2-[2-chloro-1-(4-chlorobenzyl)-5-methyl-1H-indol-3-yl]-N-(2-chloropyridin-4-yl)-2-oxoacetamide
2-[2-chloro-1-(4-chlorobenzyl)-5-methyl-1H-indol-3-yl]-N-(2-methoxypyridin-4-yl)-2-oxoacetamide
2-[2-chloro-1-(4-chlorobenzyl)-5-methyl-1H-indol-3-yl]-N-(3-chlorophenyl)-2-oxoacetamide
2-[2-chloro-1-(4-chlorobenzyl)-5-methyl-1H-indol-3-yl]-N-(3-fluorophenyl)-2-oxoacetamide
2-[2-chloro-1-(4-chlorobenzyl)-5-methyl-1H-indol-3-yl]-N-(3-methoxyphenyl)-2-oxoacetamide
2-[2-chloro-1-(4-fluorobenzyl)-5-methoxy-1H-indol-3-yl]-2-oxo-N-pyridin-4-ylacetamide
2-[2-chloro-1-(4-fluorobenzyl)-5-methoxy-1H-indol-3-yl]-2-oxo-N-pyrimidin-4-ylacetamide
2-[2-chloro-1-(4-fluorobenzyl)-5-methoxy-1H-indol-3-yl]-N-(2-chloropyridin-4-yl)-2-oxoacetamide
2-[2-chloro-1-(4-fluorobenzyl)-5-methoxy-1H-indol-3-yl]-N-(2-methoxypyridin-4-yl)-2-oxoacetamide
2-[2-chloro-1-(4-fluorobenzyl)-5-methoxy-1H-indol-3-yl]-N-(3-chlorophenyl)-2-oxoacetamide
2-[2-chloro-1-(4-fluorobenzyl)-5-methoxy-1H-indol-3-yl]-N-(3-fluorophenyl)-2-oxoacetamide
2-[2-chloro-1-(4-fluorobenzyl)-5-methoxy-1H-indol-3-yl]-N-(3-methoxyphenyl)-2-oxoacetamide
2-[5-chloro-1-(4-chlorobenzyl)-2-methyl-1H-indol-3-yl]-N-(2-methoxypyridin-4-yl)-2-oxoacetamide
2-[5-chloro-1-(4-fluorobenzyl)-2-methyl-1H-indol-3-yl]-N-(2-methoxypyridin-4-yl)-2-oxoacetamide
2-[5-fluoro-1-(4-fluorobenzyl)-2-methyl-1H-indol-3-yl]-N-(2-methoxypyridin-4-yl)-2-oxoacetamide
2-[5-methoxy-1-(4-methoxybenzyl)-2-methyl-1H-indol-3-yl]-N-(2-methoxypyridin-4-yl)-2-oxoacetamide
2-[5-methoxy-2-methyl-1-(4-methylbenzyl)-1H-indol-3-yl]-N-(2-methoxypyridin-4-yl)-2-oxoacetamide
2-{5-methoxy-2-methyl-1-[4-(trifluoromethoxy)benzyl]-1H-indol-3-yl}-N-(2-methoxypyridin-4-yl)-2-oxoacetamide
2-{5-methoxy-2-methyl-1-[4-(trifluoromethyl)benzyl]-1H-indol-3-yl}-N-(2-methoxypyridin-4-yl)-2-oxoacetamide
N-(2-chloropyridin-4-yl)-2-[1-(2,4-dichlorobenzyl)-5-methoxy-2-methyl-1H-indol-3-yl]-2-oxoacetamide
N-(2-chloropyridin-4-yl)-2-[1-(4-fluorobenzyl)-5-methoxy-2-methyl-1H-indol-3-yl]-2-oxoacetamide
N-(2-chloropyridin-4-yl)-2-[5-methoxy-1-(4-methoxybenzyl)-2-methyl-1H-indol-3-yl]-2-oxoacetamide
N-(3-chlorophenyl)-2-[1-(4-fluorobenzyl)-5-methoxy-2-methyl-1H-indol-3-yl]-2-oxoacetamide
N-(3-chlorophenyl)-2-[5-methoxy-1-(4-methoxybenzyl)-2-methyl-1H-indol-3-yl]-2-oxoacetamide
N-(3-fluorophenyl)-2-[5-methoxy-1-(4-methoxybenzyl)-2-methyl-1H-indol-3-yl]-2-oxoacetamide.

In other embodiments of the above methods, compositions, kits and uses, said FAAH inhibitor is a compound of formula XI or a pharmaceutically acceptable salt thereof:

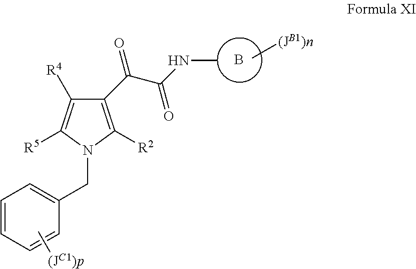

wherein:
ring B is selected from the group consisting of phenyl and a 5-6 membered monocyclic heteroaryl ring, wherein said monocyclic heteroaryl ring contains up to 3 ring heteroatoms selected from the group consisting of N, O or S;
n is an integer selected from the group consisting of 0, 1, 2 and 3;
each J^{B1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆ haloalkoxy and C₃₋₆ cycloalkoxy;
each J^{C1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆ haloalkoxy and C₃₋₆ cycloalkoxy;
p is an integer selected from the group consisting of 0, 1, 2 and 3;
R² is selected from the group consisting of halogen, —NO₂, —CN, C₁₋₆ aliphatic, phenyl, a 5-6 membered heteroaryl ring and a C₃₋₇ cycloalkyl, wherein said C₁₋₆ aliphatic, phenyl, 5-6 membered heteroaryl ring and C₃₋₇ cycloalkyl is optionally substituted by up to three instances of halogen;
R⁴ is selected from the group consisting of hydrogen, halogen, —CN, C₁₋₆ aliphatic, a C₃₋₇ cycloaliphatic ring, a 5-6 membered heteroaryl ring, phenyl, —OR^{Y} and —SR^{Y};
R⁵ is selected from the group consisting of hydrogen, halogen, —CN, C₁₋₆ aliphatic, a C₃₋₇ cycloaliphatic ring, a 5-6 membered heteroaryl ring, phenyl, —OR^{Y} and —SR^{Y} wherein said C₁₋₆ aliphatic, C₃₋₇ cycloaliphatic ring, 5-6 membered heteroaryl ring, and phenyl is optionally substituted with up to three instances of halogen, C₁₋₄ alkyl, C₁₋₄ haloalkyl, C₁₋₄ alkoxy or C₁₋₄ haloalkoxy; or

R⁴ and R⁵, together with the two carbon atoms to which they are attached, form a C₅₋₈ cycloaliphatic ring, a 5-8 membered heterocyclic ring or a 5 membered heteroaryl ring;

wherein said heterocyclic and heteroaryl ring formed by R⁴ and R⁵ contains up to three heteroatoms selected from the group consisting of N, O or S, and wherein said cycloaliphatic, heterocyclic and heteroaryl rings formed by R⁴ and R⁵ is optionally substituted by up to 3 instances of halogen, C₁₋₄ alkyl, C₁₋₄ haloalkyl, C₁₋₄ alkoxy or C₁₋₄ haloalkoxy; and

each R^{Y} is independently selected from the group consisting of C₁₋₆ aliphatic, C₃₋₇ cycloaliphatic, a 5-6 membered heteroaryl ring and phenyl, wherein each R^{Y} is optionally substituted by up to six instances of halogen, C₁₋₄ alkyl, C₁₋₄ haloalkyl, C₁₋₄ alkoxy or C₁₋₄ haloalkoxy.

In some embodiments of the above methods, compositions, kits and uses, the compound is not:

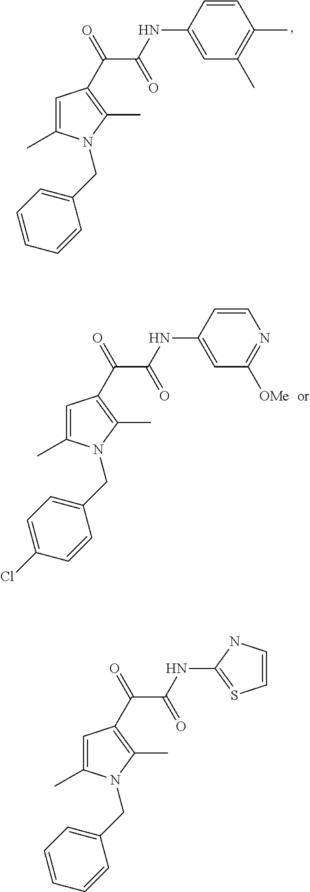

In other embodiments of the above methods, compositions, kits and uses, Ring B is an optionally substituted ring selected from the group consisting of phenyl, pyridine, pyrimidine, pyrazine, pyridazine, pyrrole, imidazole, pyrazole, furan, thiophene, triazole, tetrazole, thiazole, oxathiazole and oxazole in said compounds of Formula XI. In other embodiments, Ring B is an optionally substituted pyridine or an optionally substituted phenyl in said compound. In still other embodiments, Ring B is an optionally substituted pyridine in said compound. In further embodiments Ring B is an optionally substituted phenyl in said compound.

In some embodiments of the above methods, compositions, kits and uses, n is selected from the group consisting of 0 and 1 in said compounds of Formula XI.

In some embodiments of the above methods, compositions, kits and uses, J^{B1} is independently selected from the group consisting of halogen, C₁₋₄ alkyl, cyclopropyl, cyclopropyloxy, C₁₋₄ haloalkyl, C₁₋₄ alkoxy and C₁₋₄ haloalkoxy in said compound of Formula XI. In other embodiments, each J^{B1} is independently selected from the group consisting of halogen, methyl, ethyl, propyl, isopropyl, trifluoromethyl, methoxy, trifluoromethoxy, ethoxy, propyloxy and isopropyloxy in said compound. In still other embodiments,

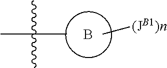

is selected from the group consisting of phenyl, 3-chlorophenyl, 3-pyridine, 4-pyridine and 3-methoxy-4-pyridine in said compound.

In some embodiments of the above methods, compositions, kits and uses, p is selected from the group consisting of 0, 1 and 2 in said compounds of Formula XI.

In some embodiments of the above methods, compositions, kits and uses, each J^{C1} is independently selected from the group consisting of halogen, C₁₋₄ alkyl, C₁₋₄ haloalkyl, cyclopropyl, cyclopropyloxy, C₁₋₄ alkoxy and C₁₋₄ haloalkoxy in said compound of Formula XI. In other embodiments, each J^{C1} is independently selected from the group consisting of halogen, methyl, ethyl, propyl, isopropyl, trifluoromethyl, methoxy, trifluoromethoxy, ethoxy, propyloxy and isopropyloxy in said compound. In still other embodiments, each J^{C1} is halogen in said compound. In further embodiments, J^{C1} is chlorine and p is 1 or 2 in said compound. In yet other embodiments, J^{C1} is fluorine and p is 1 in said compound. In yet other embodiments, J^{C1} is methoxy and p is 1 in said compound.

In some embodiments of the above methods, compositions, kits and uses, R² is selected from the group consisting of halogen, —NO₂, —CN, C₁₋₆ aliphatic or phenyl, wherein, each C₁₋₆ aliphatic and phenyl is optionally substituted with up to three instances of halogen in said compound of Formula XI. In other embodiments, R² is methyl, ethyl, propyl, isopropyl, butyl, isobutyl, t-butyl, pentyl or hexyl in said compound. In still other embodiments, R² is methyl in said compound. In further embodiments, R² is phenyl in said compound.

In some embodiments of the above methods, compositions, uses and kits, R⁴ is hydrogen, C₁₋₄ alkyl, a 5-6 membered heteroaryl or phenyl in said compound of Formula XI. In other embodiments, R⁴ is hydrogen in said compound. In still other embodiments, R⁴ is phenyl in said compound.

In some embodiments of the above methods, compositions, uses and kits, R⁵ is a C₁₋₄ alkyl, a 5-6 membered heteroaryl or phenyl in said compound of Formula XI. In other embodiments, R⁵ is methyl in said compound. In still other embodiments, R⁵ is phenyl in said compound.

In some embodiments of the above methods, compositions, kits and uses, R⁴ and R⁵, together with the two carbon atoms to which they are attached, form a C₅₋₈ cycloaliphatic ring, a 5-8 membered heterocyclic ring or a 5 membered heteroaryl ring, wherein said cycloaliphatic, heterocyclic and heteroaryl ring formed by R⁴ and R⁵ is optionally substituted with up to 3 instances of halogen, C₁₋₂ alkyl, C₁₋₂ haloalkyl, C₁₋₂ alkoxy or C₁₋₂ haloalkoxy in said compound of Formula XI. In other embodiments, R⁴ and R⁵, together with the two carbon atoms to which they are attached, form an optionally substituted C₅₋₈ cycloaliphatic ring in said compound. In still other embodiments, R⁴ and R⁵, together with the two carbon atoms to which they are attached, form the fused ring:

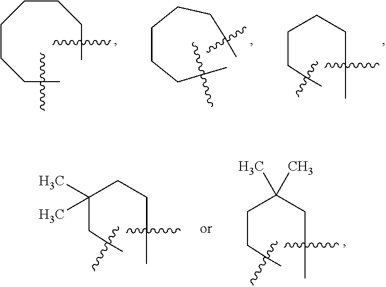

in said compound. In further embodiments, R⁴ and R⁵, together with the two carbon atoms to which they are attached, form an optionally substituted 5 membered heteroaryl ring in said compound. In yet other embodiments, R⁴ and R⁵, together with the two carbon atoms to which they are attached, form an optionally substituted thiophene ring in said compound. In yet other embodiments, R⁴ and R⁵, together with the pyrrole ring to which they are attached, form

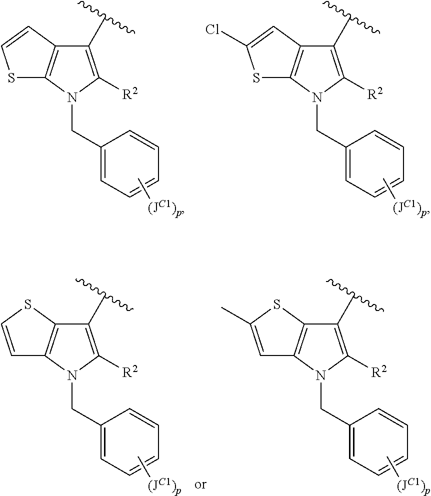

in said compound.

In some embodiments of the above methods, compositions, combinations, kits and uses, said FAAH inhibitor is represented by Formula XII or a pharmaceutically acceptable salt thereof:

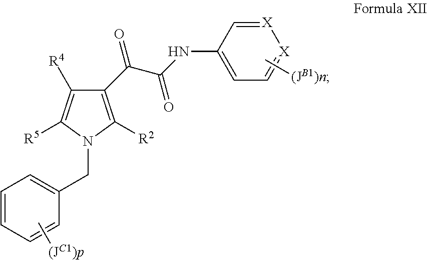

wherein each X is independently selected from the group consisting of C and N and the remaining of the variables are as described for Formula XI.

In some embodiments of the above methods, compositions, kits and uses, said FAAH inhibitor is represented by Formula XIII, or a pharmaceutically acceptable salt thereof:

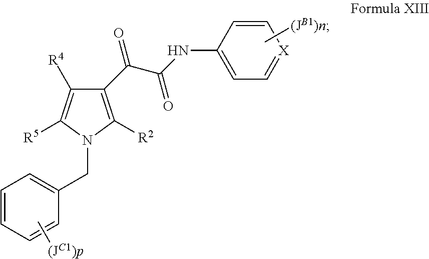

wherein n is selected from the group consisting of 0 or 1 and J^{B1} is selected from the group consisting of halogen and methoxy and wherein the remaining variables are as described above for Formula XI.

In some embodiments of the above methods, compositions, kits and uses, said FAAH inhibitor is represented by Formula XIV, or a pharmaceutically acceptable salt thereof:

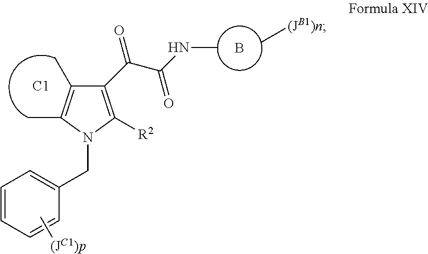

wherein ring C1 is an optionally substituted C₅₋₈ cycloaliphatic ring and the remaining variables are as described above for Formula XI. In other embodiments ring C1 is optionally substituted with up to two instances of methyl in said compound.

In some embodiments of the above methods, compositions, kits and uses, said FAAH inhibitor is represented by Formula XV, or a pharmaceutically acceptable salt thereof;

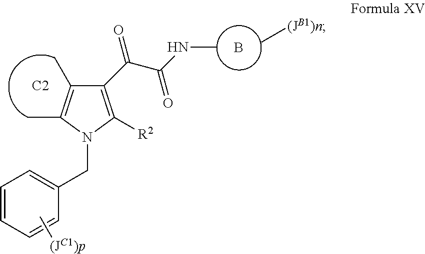

wherein ring C2 is an optionally substituted 5 membered heterocyclic ring. In other embodiments, ring C2 is an optionally substituted thiophene ring in said compound of Formula XV and the remaining variables are as described above for Formula XI. In other embodiments, ring C2 is optionally substituted with up to two instances of methyl or halogen in said compound.

In some embodiments of the above methods, compositions, kits and uses, said FAAH inhibitor is selected from those depicted below, or a pharmaceutically acceptable salt thereof:

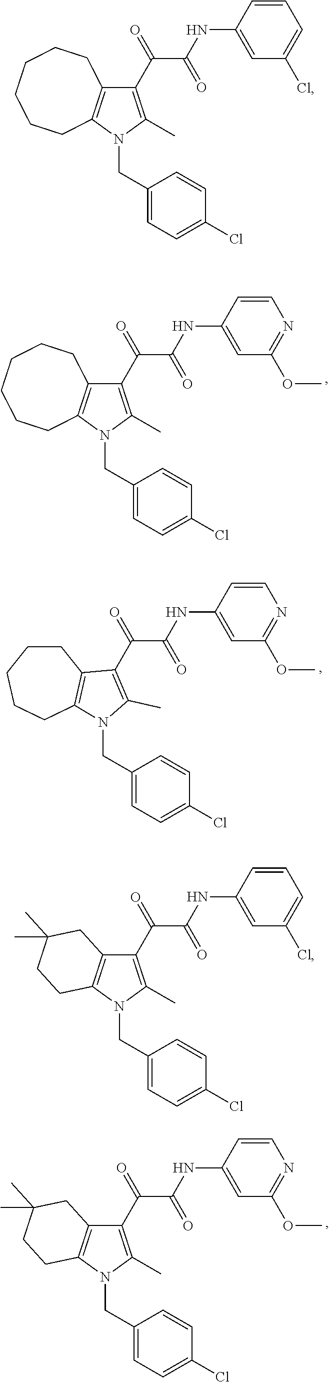

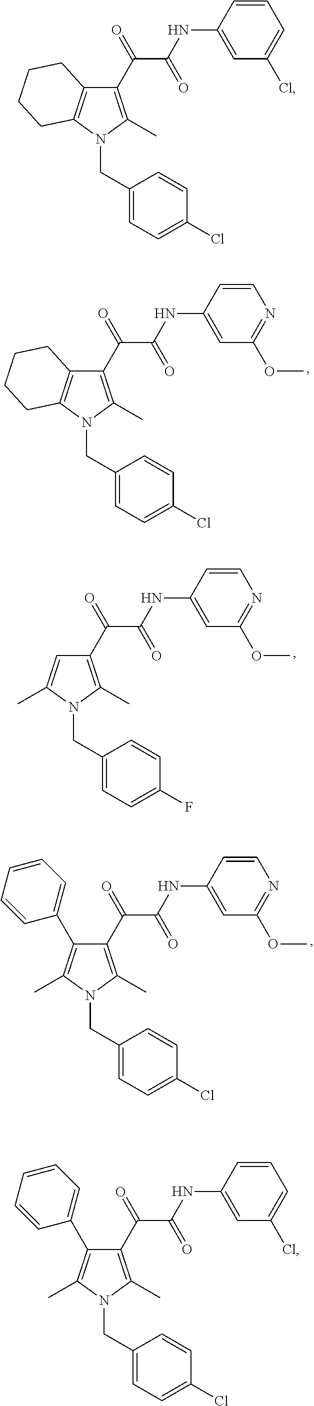

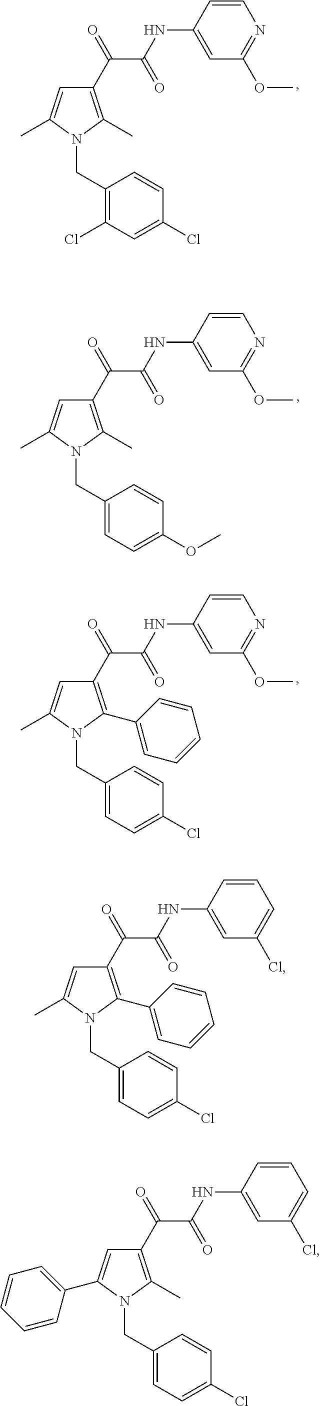

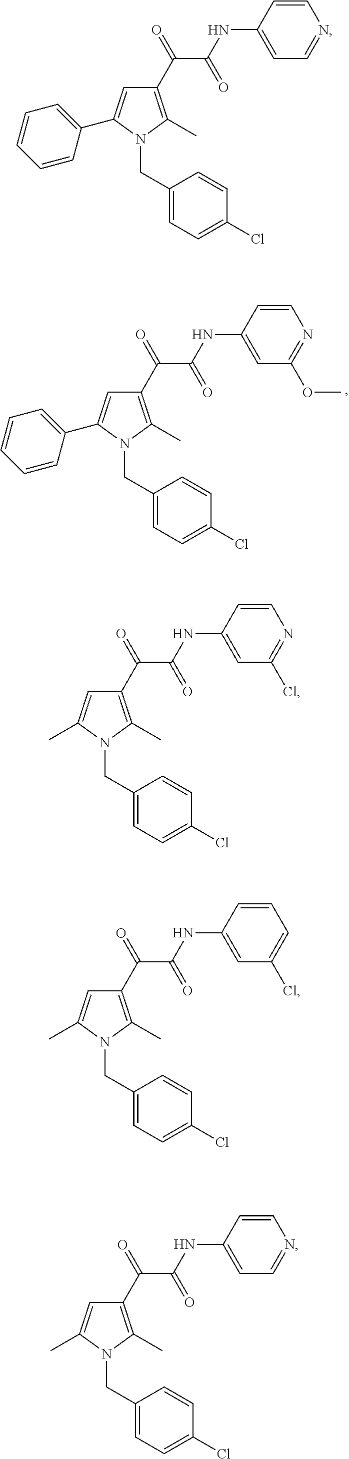

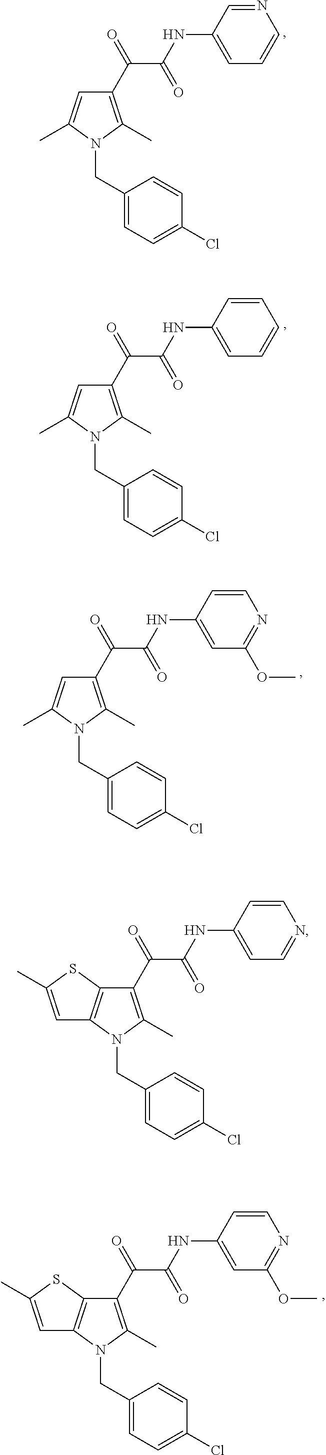

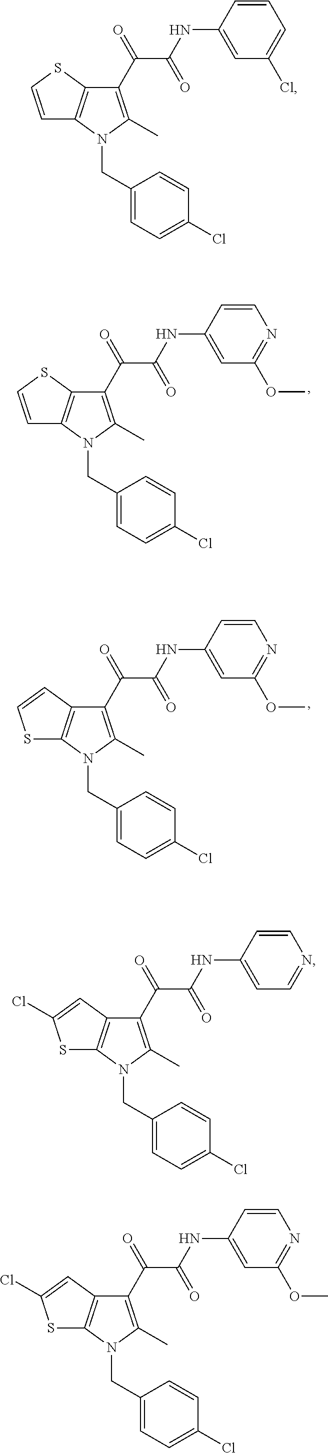

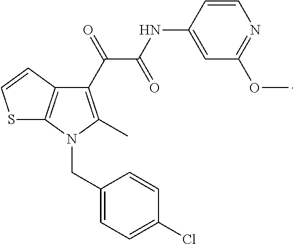 [SMILES: COc1cc(NC(=O)C(=O)c2c(C)n(Cc3ccc(Cl)cc3)c3sccc23)ccn1]

In some embodiments of the above methods and uses, the FAAH inhibitor is administered before a symptom of abdominal, visceral or pelvic pain develops in said patient. In other embodiments, it is administered after the symptom develops. In further embodiments, the FAAH inhibitor is administered prior to, at the same time or after the initiation of treatment with another therapeutic agent. In some embodiments of the above methods and uses, the FAAH inhibitor is administered after one or more symptoms of abdominal pain or IBS develops in said patient.

In some embodiments of the above methods and uses, the additional therapeutic agent and the FAAH inhibitor are administered simultaneously. In other embodiments of the above methods and uses, the additional therapeutic agent and the FAAH inhibitor are administered sequentially or separately.

In some embodiments, the above pharmaceutical compositions or kits comprise (a) a FAAH inhibitor as discussed above, a pharmaceutically acceptable salt thereof, a pharmaceutically acceptable solvate (e.g., hydrate) or co-crystal of the compound or salt thereof, and (b) a pharmaceutically acceptable carrier, vehicle or adjuvant. In some embodiments, the pharmaceutical composition or kit comprises (a) an additional therapeutic agent as discussed above, a pharmaceutically acceptable salt thereof, a pharmaceutically acceptable solvate (e.g., hydrate) or co-crystal of the compound or salt thereof, and (b) a pharmaceutically acceptable carrier, vehicle or adjuvant. In some embodiments, the pharmaceutical composition comprises (i) a FAAH inhibitor as discussed above, or a pharmaceutically acceptable salt thereof, (ii) an additional therapeutic agent as discussed above, or a pharmaceutically acceptable salt thereof, and (iii) a pharmaceutically acceptable carrier, vehicle or adjuvant. In a further embodiment, the pharmaceutical composition further comprises at least one additional therapeutic agent.

### Pharmaceutically Acceptable Salts, Co-Forms and Pro-Drugs

In some embodiments of the methods, uses, pharmaceutical compositions or kits, the FAAH inhibitor may be provided as (i) the compound itself (e.g., as the free base); (ii) a pharmaceutically acceptable salt of the compound; (iii) a pharmaceutically acceptable solvate (e.g., hydrate) or co-crystal of the FAAH inhibitor compound or salt thereof; or (iv) part of a pharmaceutical composition. In some embodiments of the above methods, uses, pharmaceutical compositions and kits, the additional therapeutic agent may be provided as (i) the compound itself (e.g., as the free base); (ii) a pharmaceutically acceptable salt of the compound; (iii) a pharmaceutically acceptable solvate (e.g., hydrate) or co-crystal of the therapeutic agent or salt thereof; or (iv) part of a pharmaceutical composition.

The phrase “pharmaceutically acceptable salt,” as used herein, refers to pharmaceutically acceptable organic or inorganic salts of a compound described herein. For use in medicine, the salts of the compounds described herein will be pharmaceutically acceptable salts. Other salts may, however, be useful in the preparation of the compounds described herein or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound described herein is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound described herein is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated herein by reference in its entirety.

In addition to the compounds described herein and their pharmaceutically acceptable salts, pharmaceutically acceptable solvates (e.g., hydrates) and co-crystals of these compounds and salts may also be employed in compositions to treat or prevent the herein identified disorders.

As used herein, the term “pharmaceutically acceptable solvate,” is a solvate formed from the association of one or more pharmaceutically acceptable solvent molecules to one of the compounds described herein. As used herein, the term “hydrate” means a compound described herein or a salt thereof that further includes a stoichiometric or non-stoichiometric amount of water bound by non-covalent intermolecular forces. The term solvate includes hydrates (e.g., hemihydrate, monohydrate, dihydrate, trihydrate, tetrahydrate, and the like).

“Pharmaceutically acceptable co-crystals” result when a pharmaceutically active compound crystallizes with another material (e.g. a carboxylic acid, a 4,4′-bipyridine or an excipient) that is also a solid at room temperature. Some pharmaceutically acceptable excipients are given, for example by the GRAS (Generally Regarded As Safe) and the EAFUS (Everything Added to Food in the U.S.) databases maintained by the U.S. Food and Drug Administration (F.D.A.).

### Pharmaceutical Compositions and Methods of Administration

The compounds herein disclosed, and their pharmaceutically acceptable salts, solvates, co-crystals and pro-drugs thereof may be formulated as pharmaceutical compositions or “formulations”.

A typical formulation is prepared by mixing a compound described herein, or a pharmaceutically acceptable salt, solvate, co-crystal or pro-drug thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound described herein is being formulated. Solvents are generally selected based on solvents recognized by persons skilled in the art as safe (e.g., one described in the GRAS database) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

The formulations may be prepared using conventional dissolution and mixing procedures. For example, the bulk drug substance (i.e., one or more of the compounds described herein, a pharmaceutically acceptable salt, solvate, co-crystal or pro-drug thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8.

A compound described herein or a pharmaceutically acceptable salt, solvate, co-crystal or pro-drug thereof is typically formulated into pharmaceutical dosage forms to provide an easily controllable dosage of the drug and to enable patient compliance with the prescribed regimen. Pharmaceutical formulations of compounds described herein, or a pharmaceutically acceptable salt, solvate, co-crystal or pro-drug thereof, may be prepared for various routes and types of administration. Various dosage forms may exist for the same compound. The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total composition (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 ml/hr can occur.

The pharmaceutical compositions described herein will be formulated, dosed, and administered in a fashion, i.e., amounts, concentrations, schedules, course, vehicles, and route of administration, consistent with good medical practice. Factors for consideration in this context include the particular disorder being treated, the particular human or other mammal being treated, the clinical condition of the individual patient, the cause of the disorder, the site of delivery of the agent, the method of administration, the scheduling of administration, and other factors known to medical practitioners, such as the age, weight, and response of the individual patient.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease or disorder or one or more of its symptoms.

The term “prophylactically effective amount” refers to an amount effective in preventing or substantially lessening the chances of acquiring a disorder or in reducing the severity of the disorder or one or more of its symptoms before it is acquired or before the symptoms develop.

In some embodiments, a prophylactically effective amount of a FAAH inhibitor is one that prevents the occurrence or reoccurrence of pain or irritable bowel syndrome. In further embodiments, a prophylactically effective amount of a FAAH inhibitor is one that prevents the occurrence or reoccurrence of pain, for example, pelvic pain, bladder pain, cancer pain, intestinal cramps, abdominal discomfort, abdominal pain, bowel pain, pancreas pain, stomach pain, gastrointestinal pain, referred pain, rectal pain, visceral pain, tissue pain, discomfort associated with irritable bowel syndrome, and the like.

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”.

“Sustained-release preparations” are the most common applications of controlled release. Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent disaggregation to fine particles. This provides a larger surface area exposed to the dissolution medium, resulting in a faster dissolution rate.

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. In particular embodiments, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound described herein that are suitable for oral administration may be prepared as discrete units such as tablets, pills, troches, lozenges, aqueous or oil suspensions, dispersible powders or granules, emulsions, hard or soft capsules, e.g. gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a water-soluble carrier such as polyethyleneglycol or an oil medium, for example peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g., for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending a compound described herein in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds described herein contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable drug-depot forms are made by forming microencapsuled matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Drug-depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl ester wax, cetearyl alcohol, 2 octyldodecanol, benzyl alcohol and water.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, between 0.075% and 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds described herein may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds described herein include Tween™-60, SpanM-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration may have a mean particle size in the range of, for example, 0.1 to 500 microns (including particles with a mean particle size in the range between 0.1 and 500 microns in increments such as 0.5, 1, 30, 35 microns, etc) which may be administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In another aspect, a compound described herein or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Therapeutic Methods

As used herein, the terms “subject” and “patient” are used interchangeably. The terms “subject” and “patient” refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of a condition that can be treated with a FAAH inhibitor, or the amelioration of one or more symptoms (preferably, one or more discernible symptoms) of said condition, resulting from the administration of one or more therapies (e.g., one or more therapeutic agents such as a compound or composition of the invention). In specific embodiments, the terms “treat”, “treatment” and “treating” refer to the amelioration of at least one measurable physical parameter of condition that can be treated with a FAAH inhibitor. In other embodiments the terms “treat”, “treatment” and “treating” refer to the inhibition of the progression of said condition, either physically by, e.g., stabilization of a discernible symptom, physiologically by, e.g., stabilization of a physical parameter, or both.

In some embodiments, the terms “treat”, “treatment” and “treating” as it pertains to the use of a FAAH inhibitor refers to ameliorating or alleviating pain in a patient that exhibits these symptoms.

As used herein, the terms “prevent”, “preventing” and “prevention” with regard to a disorder or disease refer to averting the cause and/or effects of a disease or disorder prior to the disease or disorder manifesting itself. The terms “prophylaxis” or “prophylactic use”, as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject. “Abdominal pain”, “visceral pain” or “pelvic pain” includes, for example:

(a) Gastrointestinal pain such as stomach pain, rectal pain, bowel pain, intestinal pain, intestinal cramps; pain and/or discomfort associated with gastroenteritis, appendicitis, gastritis, functional dyspepsia, esophagitis, diverticulitis, Crohn's disease, ulcerative colitis, microscopic colitis, hernias and other types of GI obstructions, abdominal angina, blood vessel compression, celiac disease and celiac artery compression syndrome, peptic ulcer, lactose intolerance, food allergies, Irritable bowel syndrome (IBS), BS-c, IBS-d, IBS-a, irritable bowel disease or digestive disorders; pain associated with gastro-esophageal reflux disease.

(b) Pain of the bile system, for example associated with cholecystitis, cholangitis, or tumors.

(c) Liver pain, such as, for example, pain associated with hepatitis or liver abscesses.

(d) Pancreatic pain, such as, for example, pain associated with pancreatitis or gallbladder pain.

(e) Renal or urological pain, such as, for example, kidney pain, ureter pain, bladder pain, prostate pain; pain associated with pyelonephritis, bladder infection, kidney stones, urolithiasis, urinary retention, interstitial cystitis, prostatitis, painful bladder syndrome, inflammatory pelvic disease, tumors or left renal vein entrapment.

(f) Gynecological or obstetric pain, such as, for instance, ovarian pain, uterine pain, labor pain, vulvar pain, vaginal pain or menstrual cramps; pain associated with pelvic inflammatory disease, post-menopausal pelvic pain, ovarian torsion, menstruation, dysmenorrhea, dyspareuinia, endometriosis, vulvodynia, fibroids, ovarian cysts, or ovarian cancer.

(g) Cardiac pain.

(h) Referred pain, such as pain referred from the thorax as a result of pneumonia, pulmonary embolism or ischemic heart disease, etc; for example referred from the site of pain as a result of radiculitis; and for example referred from the genitals as a result of testicular torsion.

(i) Pain resulting from infections by parasites, bacteria or viruses, surgery, trauma, cancer, exposure to noxious chemicals or medications.

“Visceral pain” includes, for example, pain associated with pancreatitis, peptic ulcer, interstitial cystitis, renal colic, angina, dysmenorrhea, menstrual cramps, menstruation, irritable bowel syndrome (IBS), myocardial ischemia, and non-ulcer dyspepsia. Visceral pain also includes gynecological pain, urinary bladder pain, kidney pain, non-cardiac chest pain, and chronic pelvic pain.

“Cancer pain” can be induced by or associated with tumors such as lymphatic leukemia, Hodgkin's disease, malignant lymphoma, osteosarcoma, bone cancer, lymphogranulomatoses, lymphosarcoma, solid malignant tumors, and extensive metastases. Chemotherapy pain is a side effect of chemotherapy treatments.

“Pelvic pain” can result, for example, from endometriosis, neurological hypersensitivity due to infections or post-infection, exaggerated bladder, bowel or uterine pain sensivity, ovarian cysts, uterine leiomyoma, ovarian torsion, appendicitis, pelvic girdle pain, dysmenorrhea, pelvic inflammatory disease, ovarian abnormalities, colitis, proctitis, or diseases of the prostate.

Compounds and compositions of the invention are also useful for veterinary treatment of companion animals, exotic animals and farm animals, including, without limitation, dogs, cats, mice, rats, hamsters, gerbils, guinea pigs, rabbits, horses, pigs and cattle.

### Combination Therapies

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one agent may be prior to, concurrent to, or subsequent to the administration of the other agent.

When co-administered with other agents, e.g., when co-administered with another pain medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.001 to about 100 mg/kg body weight/day, from about 0.001 to about 50 mg/kg body weight/day, from about 0.001 to about 30 mg/kg body weight/day, from about 0.001 to about 10 mg/kg body weight/day.

When “combination therapy” is employed, an effective amount can be achieved using a first amount of a compound described herein or a pharmaceutically acceptable salt, solvate (e.g., hydrate), co-crystal or pro-drug thereof and a second amount of an additional suitable therapeutic agent (e.g. an agent to treat pain).

In one embodiment of this invention, the compound described herein and the additional therapeutic agent, are each administered in an effective amount (i.e., each in an amount which would be therapeutically effective if administered alone). In another embodiment, the compound described herein and the additional therapeutic agent, are each administered in an amount which alone does not provide a therapeutic effect (a sub-therapeutic dose). In yet another embodiment, the compound described herein can be administered in an effective amount, while the additional therapeutic agent is administered in a sub-therapeutic dose. In still another embodiment, the compound described herein can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When co-administration involves the separate administration of the first amount of a compound described herein and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound described herein and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks prior), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks subsequent) the administration of a second therapy (e.g., a therapeutic agent) to a subject.

### Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for cotherapy use, a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or other compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

All references provided in the Examples are herein incorporated by reference in their entirety. As used herein, all abbreviations, symbols and conventions are consistent with those used in the contemporary scientific literature. See, e.g. Janet S. Dodd, ed., The ACS Style Guide: A Manual for Authors and Editors, 2^{nd} Ed., Washington, D.C.: American Chemical Society, 1997, herein incorporated by reference in its entirety.

### Colonic Hyperalgesia Animal Models

Hypersensitivity to colorectal distension is common in patients with IBS and may be responsible for the major symptom of pain. Both inflammatory and non-inflammatory animal models of visceral hyperalgesia to distension have been developed to investigate the effect of compounds on visceral pain in IBS and include, among others, the basal sensitivity to colorectal distension model, the partial restraint stress-induced hyperalgesia model and the cortagine-induced visceral hypersensitivity model. The Examples described below were conducted using the FAAH inhibitors [3′-(aminocarbonyl) biphenyl-3-yl cyclohexyl carbamate](URB597) and the compounds designated as compound A (2-(1-(4-chlorobenzyl)-5-methoxy-2-methyl-1H-indol-3-yl)-N-(3-chlorophenyl)-2-oxoacetamide) and compound B (2-(1-(4-chlorobenzyl)-2,5-dimethyl-1H-indol-3-yl)-N-(2-methoxypyridin-4-yl)-2-oxoacetamide).

### Example 1

### Effect of FAAH Inhibitors on Basal Sensitivity to Colorectal Distension Model

Female Wistar rats (weighing 200-250 g each), were surgically prepared for electromyography as previously described (Morteau et al., Dig. Dis. Sci. 1994: 39:1239-48; “Guanylate cyclase C-mediated antinociceptive effects of linaclotide in rodent models of visceral pain”, Eutamene H, Bradesi S, Larauche M, Theodorou V, Beaufrand C, Ohning G, Fioramonti J, Cohen M, Bryant A P, Kurtz C, Currie M G, Mayer E A, Bueno L, Neurogastroenterology and motility, 2010, Volume: 22 p. 312-322; “Stress-induced changes in intestinal transit in the rat: a model for irritable bowel syndrome”, Williams C L, Villar R G, Peterson J M, Burks T F, Gastroenterology—1988 p. 611-21) and were used to evaluate the effects of the FAAH inhibitors, compound A and compound B, on basal sensitivity to colorectal distension.

The basal sensitivity to colorectal distension was determined 1 day prior to conducting the partial restraint stress-induced hyperalgesia model described in Example 2. Rats were treated with compound A (30 mg/kg in DMSO/cremophor/isotonic saline (1:1:8 v:v:v), n=8), compound B (30 mg/kg in DMSO/cremophor/isotonic saline (1:1:8 v:v:v), n=9) or vehicle only (DMSO/cremophor/isotonic saline (1:1:8 v:v:v, 1 mL, n=9) by oral gavage. The effects of the administered compounds on basal sensitivity to colorectal distension (CRD) were measured 2 hrs later.

For the CRD procedure, rats were acclimatized to restraint in polypropylene tunnels (diameter: 7 cm; length: 20 cm) periodically for several days before CRD in order to minimize recording artifacts. The balloon used for distension was 4 cm long and made from a latex condom. It was fixed on a rigid catheter taken from an embolectomy probe (Fogarty). CRD was performed by insertion of the balloon in the rectum at 1 cm from the anus. The tube was fixed at the base of the tail. Isobaric distensions were performed from 0 to 60 mmHg, with each distension step lasting 5 minutes. The first distension was performed at a pressure of 15 mmHg and an increment of 15 mmHg was added at each following step, until a maximal pressure of 60 mmHg was attained. Electromyographic recordings commenced 5 days after surgery. Electrical activity was recorded with an electroencephalograph (Mini VIII, Alvar, Paris, France) using a short time constant (0.03 sec) to remove low-frequency signals (<3 Hz) and a paper speed of 3.6 cm/minute. Isobaric distensions of the colon were performed by connecting the balloon to a computerized barostat. Colonic pressure was continuously monitored on a potentiometric recorder (L6514, Linseis, Selb, Germany) with a paper speed of 1.0 cm/minute. The number of spike bursts, corresponding to abdominal contractions, was evaluated per 5-minute period.

### Statistics

Statistical analysis was performed using a one way analysis of variance (ANOVA) followed by an unpaired two-tailed Student's t test using GraphPad Prism 4.0. Any p values<0.05 were considered significantly different. The values were expressed as mean±SEM.

### Results and Conclusions

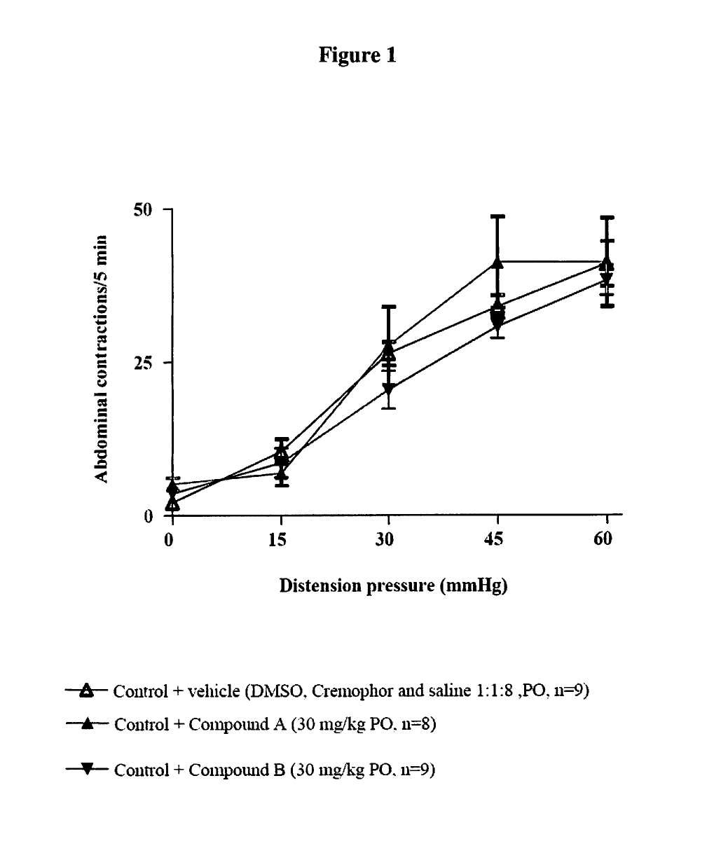

FIG. 1 shows the effects of compound A and compound B in the basal sensitivity to colorectal distension model. The number of abdominal contractions was recorded by electromyography per 5-minute period (y axis) for distension pressures of 0 mmHg, 15 mmHg, 30 mmHg, 45 mmHg and 60 mmHg (x axis). Data are mean values with SEM; the symbols [symbol] represent the results for the control+vehicle group (n=9); the symbols [symbol] represent the results for the control+compound A group (n=8); and the symbols [symbol] represent the results for the control+compound B group (n=9). The results indicate that neither compound A nor compound B alters basal sensitivity (normal responses to pain) in rats.

### Example 2

### Effect of FAAH Inhibitors in the Partial Restraint Stress-Induced Hyperalgesia Model

Female Wistar rats surgically prepared for electromyography as described in Example 1 were used to evaluate the effects of the FAAH inhibitors (a) compound A or (b) compound B, on colorectal sensitivity and compliance after a 2 hour partial restraint stress session.

Partial restraint stress (PRS), a relatively mild stress, was induced as previously described (Morteau et al. Dig. Dis. Sci. 1994: 39:1239-48; “Guanylate cyclase C-mediated antinociceptive effects of linaclotide in rodent models of visceral pain”, Eutamene H, Bradesi S, Larauche M, Theodorou V, Beaufrand C, Ohning G, Fioramonti J, Cohen M, Bryant A P, Kurtz C, Currie M G, Mayer E A, Bueno L, Neurogastroenterology and motility —2010 Volume: 22 p. 312-322; “Stress-induced changes in intestinal transit in the rat: a model for irritable bowel syndrome”, Williams C L, Villar R G, Peterson J M, Burks T F, Gastroenterology—1988 p. 611-21). Female rats were lightly anesthetized with diethyl ether and their shoulders, upper forelimbs and thoracic trunk were wrapped in a confining harness of paper tape to restrict, but not prevent body movements. Control sham-stress animals were anesthetized but not wrapped. Rats received a 2 hr PRS session where the test compound or the vehicle control was orally administered 1 hr 15 min into PRS. The effects of the administered compounds on basal sensitivity to colorectal distension (CRD) were measured 2 hrs later as described in Example 1.

### Treatments and Results

### (a) Compound A

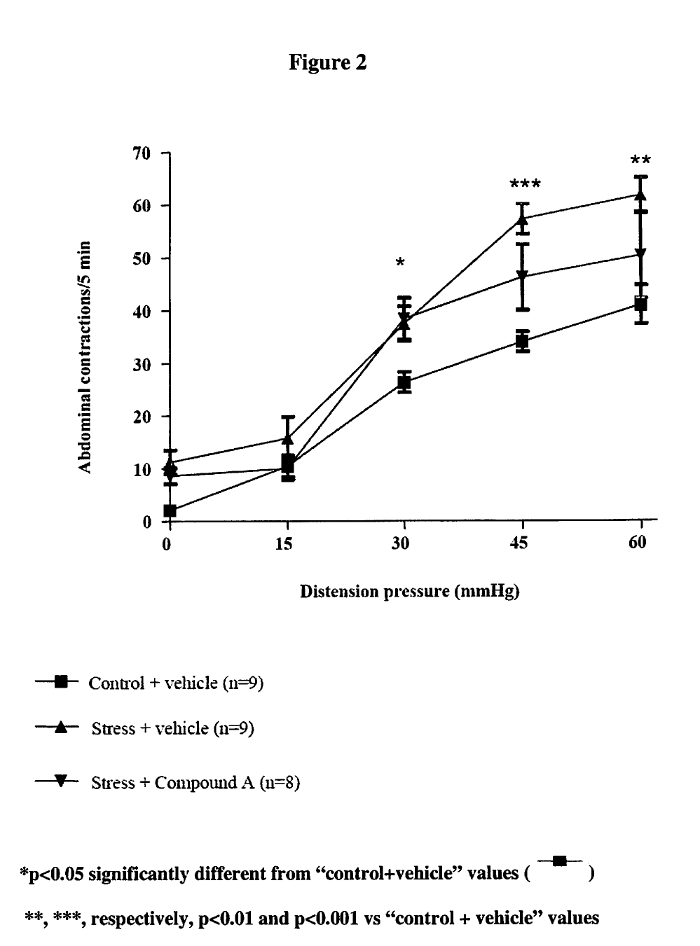

Rats were treated orally with compound A (30 mg/kg in DMSO/cremophor/isotonic saline (1:1:8 v:v:v), n=8), or vehicle only (DMSO/cremophor/isotonic saline (1:1:8 v:v:v), 1 mL, n=9) with or without 2 hour PRS sessions. The number of abdominal contractions was recorded by electromyography per 5-minute period (y axis) for each distension pressure of 0 mmHg, 15 mmHg, 30 mmHg, 45 mmHg and 60 mmHg (x axis); the [symbol] symbols represent the results for the control+vehicle group (n=9); the symbols [symbol] represent the results for the stress+vehicle group (n=9); the symbols [symbol] represent the results for the stress+compound A group (n=8). Data are mean values with SEM. Compound A showed a non-significant trend towards attenuation of abdominal contractions at the two highest distension pressures (45 and 60 mmHg) versus stress+vehicle control (FIG. 2).

### (b) Compound B

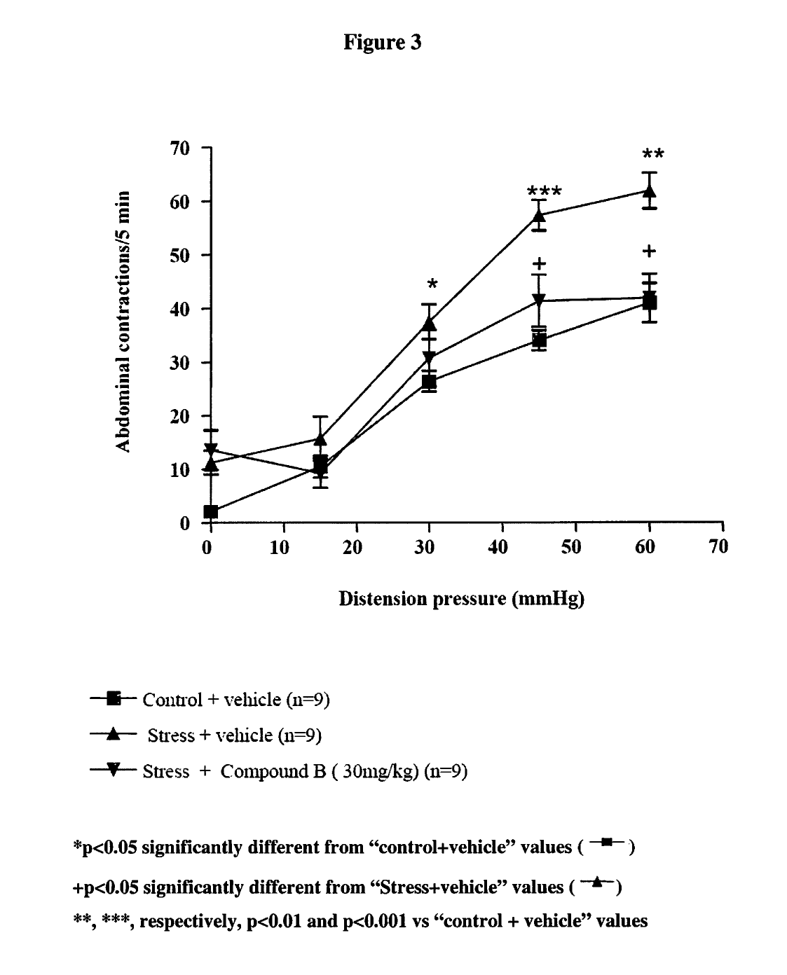

Rats were treated orally with compound B (30 mg/kg in DMSO/cremophor/isotonic saline (1:1:8 v:v:v), n=9), or vehicle only (DMSO/cremophor/isotonic saline (1:1:8 v:v:v), 1 mL, n=9) with or without 2 hour PRS sessions. The number of abdominal contractions was recorded by electromyography per 5-minute period (y axis) for each distension pressure of 0 mmHg, 15 mmHg, 30 mmHg, 45 mmHg and 60 mmHg (x axis); the symbols [symbol] represent the results for the control+vehicle group (n=9); the symbols [symbol] represent the results for the stress+vehicle group (n=9); and the symbols [symbol] represent the results for the stress+compound B group (n=9). Data are mean values with SEM. Compound B significantly reduced abdominal contractions at 45 and 60 mmHg versus stress+vehicle control (FIG. 3).

### Example 3

### Effect of FAAH Inhibitors on Cortagine-Induced Visceral Hypersensitivity in Rats

### Animals

Male Sprague Dawley (SD) rats (250-275 g, Harlan Labs, Indianapolis, Ind.) were kept under standard conditions of humidity and temperature and a 12-hour light/dark cycle (lights on 6.00 a.m.). Animals were group housed and had access to food ad libitum. Prior to the start of studies, animals were acclimatized to handling and administration of treatments (oral syringe feeding and subcutaneous injection). At the end of the experiments, animals were sacrificed by CO₂ gas inhalation followed by thoracotomy or isoflurane anesthesia followed by decapitation with appropriate approved animal protocols.

### Rodent Model

On the day of the experiments, rats were injected intraperitoneally (IP) with cortagine (10 μg/kg, 0.8 ml/kg in DMSO/cremophor/isotonic saline (1:1:8 v:v:v). Cortagine, a selective corticotropin releasing factor receptor 1 (CRF₁) agonist, prepared as described previously (Rivier et al. J. Med. Chem. 2007: 50:1668-1674), was stored in a powder form at −80° C. and prepared in sterile water (12.5 μg/ml) immediately before use. This dose had previously been established to show a significant increase in defecation, induction of diarrhea and increase in colonic motility, permeability and visceral pain in rats. (Larauche et al. Am. J. Physiol. Gastrointest. Liver Physiol. 2009: 297:G215-G227)

### Test Compounds

FAAH inhibitor compounds URB597 [3′-(aminocarbonyl)biphenyl-3-yl cyclohexyl carbamate], compound A and compound B were formulated as suspensions in DMSO/cremophor/isotonic saline (1:1:8 v:v:v). The concentration of the respective dosing FAAH inhibitor compound suspensions were 6 mg/ml for 30 mg/kg dose of compound A or compound B; 2 mg/ml for the 10 mg/kg dose of compound B; and 1.5 mg/ml for the 3 mg/kg dose of URB597. Compound A, compound B or vehicle treatment was administered to rats by per os (PO) route at dose-volume of 5 ml/kg. URB597 treatment was administered to rats by subcutaneous (SC) route at dose volume of 2 ml/kg. The vehicle for the PO route was DMSO/cremophor/isotonic saline (1:1:8 v:v:v). Vehicle (PO), URB597 (SC), compound A and compound B (PO) were administered to non-fasted rats, restrained by hand. The regimen of administration of URB597, compound A and compound B involved one delivery (PO or SC) performed 120 min before IP injection of cortagine.

### Measurement of Visceral Pain

Visceral pain was assessed using a non-invasive pressure transducer system referred to as “sensor balloon” as previously described (Larauche et al. Am. J. Physiol. Gastrointest. Liver Physiol. 2009: 297:G215-G227; Ness et al. Brain Res. 1988: 450:153-169). Briefly, in adult non-fasted SD rats, a 4-5 cm “sensor balloon” lubricated with surgical lubricant (Surgilube, Fougera, Melville) was inserted intra-anally under brief isoflurane anesthesia. The “sensor balloon” was positioned such that its distal end was 1 cm proximal to the anal verge and secured in place by tapping the balloon catheter to the tail. Rats were placed individually in Boolman's cage and allowed to recover from anesthesia and habituation. The colorectal procedure was performed using the Distender Series Ilir dual barostat (G&J Electronics Inc, Toronto, Ontario). The CRD protocol consisted of 2 CRD at 60 mmHg to unfold the balloon followed by 2 sets of CRD at 10, 20, 40 and 60 mmHg, 20 s duration, 4-min inter-stimulus interval. The intra-luminal colonic pressure (ICP) was recorded for 20 s before, during and after termination of CRD. The AUC of ICP during CRD over non-distended ICP (before CRD) was recorded as the VRM (visceromotor response, (Larauche et al. Am. J. Physiol. Gastrointest. Liver Physiol. 2009: 297:G215-G227). To examine the pressure-response relationship and adjust for inter-individual variation of the signal, ICP amplitudes were normalized as percent of the VRM response to the highest (60 mmHg) in the 1^{st} set of CRD for each rat. VRM to the 1^{st} set of CRD before treatment represents baseline VRM at different pressures of distention and is averaged for each group of rats. Rats were also visually observed for any other behavioral responses.

### Statistics

Each experimental group included 11-13 rats. Data are mean±SEM and were analyzed using ANOVA and multiple tests to assess the difference between treatment groups: the Grubb's test to determine any outlier rat (rat for which individual values appear to deviate markedly from the other members of the sample in which it occurred). Any p values<0.05 were considered significantly different.

### Experimental Protocol

All the experiments were performed on conscious male non-fasted SD rats and at the same time in the morning to avoid circadian variations that may influence experimental results.

Rats were habituated to oral gavage (once/day) and to Bollman's cages (4 h/day) for 3 consecutive days preceding the treatment. They were placed in a quiet rat room 48 h before the experimental day and were not disturbed outside of the training/gavage sessions. On the day of the experiment, at 6:30 am, animals were equipped with distension balloons and placed in Bollman's cages before being brought to the experimental room, where they were left 20 min to recover from anesthesia. At the end of the 20 min recovery period, a baseline CRD (CRD#1) of 40 min was performed at 10, 20, 40, 60 mmHg and the visceromotor response (VMR) assessed. Immediately after the end of the first CRD, rats received an oral gavage of vehicle (DMSO/cremophor/isotonic saline (1:1:8 v:v:v), 1.5 ml), compound A (30 mg/kg in vehicle), compound B (10 or 30 mg/kg in vehicle) or URB597 (3 mg/kg in vehicle, SC). Two hours after, cortagine (10 μg/kg in vehicle, IP) was injected. Fifteen minutes after cortagine injection, a second CRD (CRD#2) of 40 min was performed. At the end of the distension, the balloons were removed prior to placing the rats back into their home cages (˜15 min).

### Results: Effects of URB597, Compound a and Compound B Pre-Treatments or Vehicle on Cortagine-Induced Visceral Hypersensitivity to Colorectal Distension in Rats.

Treatments were randomized in the different groups of animals tested, so that each day of testing, each rat received a different test substance/dose.

During the experimental procedure, in the compound A group, one rat that had a normal baseline response, did not respond to the second set of CRD. The balloon and probes were working normally and the balloon was not out of the rat's colon. It is assumed that a fecal pellet may have stuck on the probe sensor and prevented recording pressure changes; thus this rat was excluded from analysis.

In the URB597 group one rat had an abnormal response to distension characterized by a total absence of response to the CRD in baseline. This rat was excluded from analysis.

In the compound B (10 mg/kg, PO) group, one rat also showed no response to the first set of CRD and was excluded from analysis.

In the compound B (30 mg/kg, PO) group, one rat lost its balloon during the course of the 2nd distension and could not be used for data analysis.

The analysis was conducted on the number of rats in each group as indicated below:
Vehicle, n=13
Compound A (30 mg/kg, PO), n=11
URB597 (3 mg/kg, SC), n=11
Compound B (10 mg/kg PO), n=11
Compound B (30 mg/kg, PO) n=11

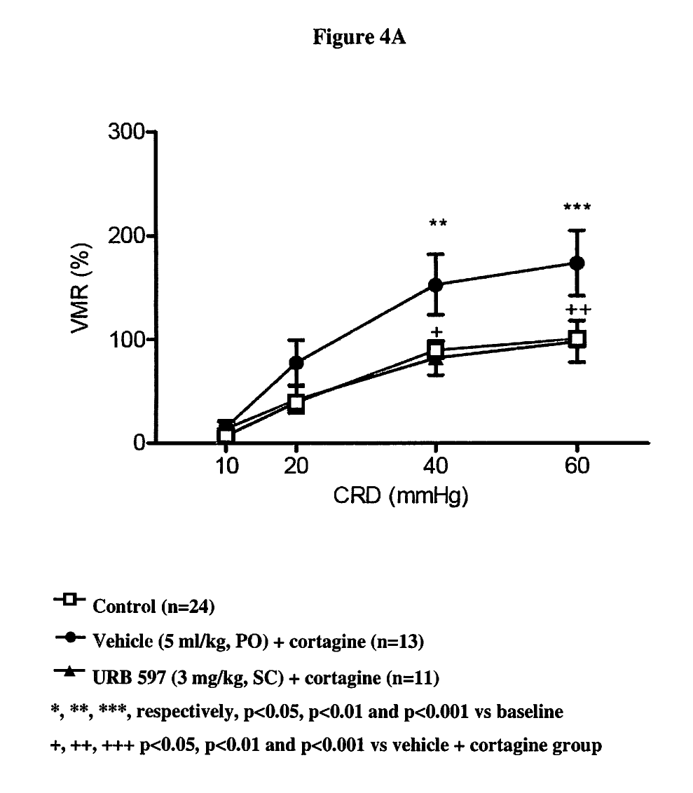

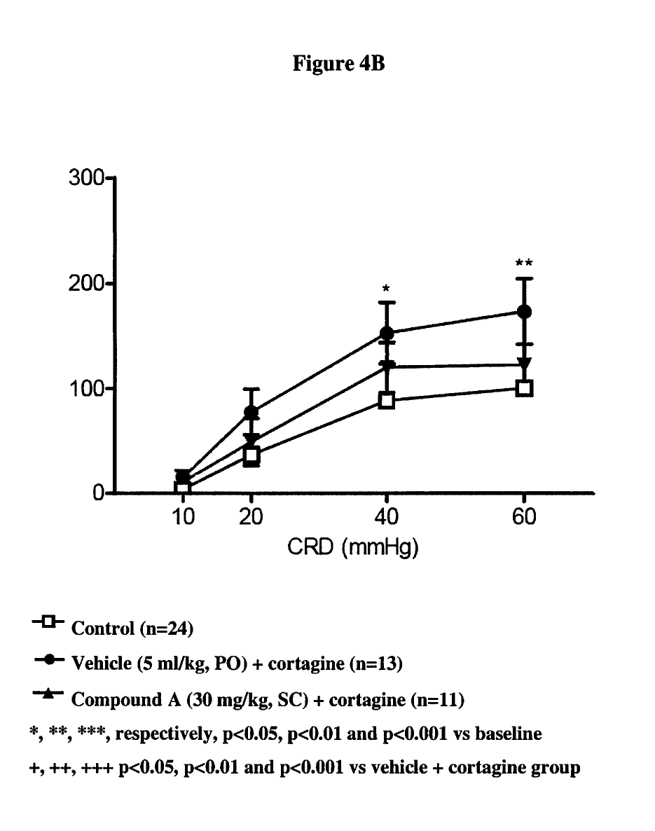

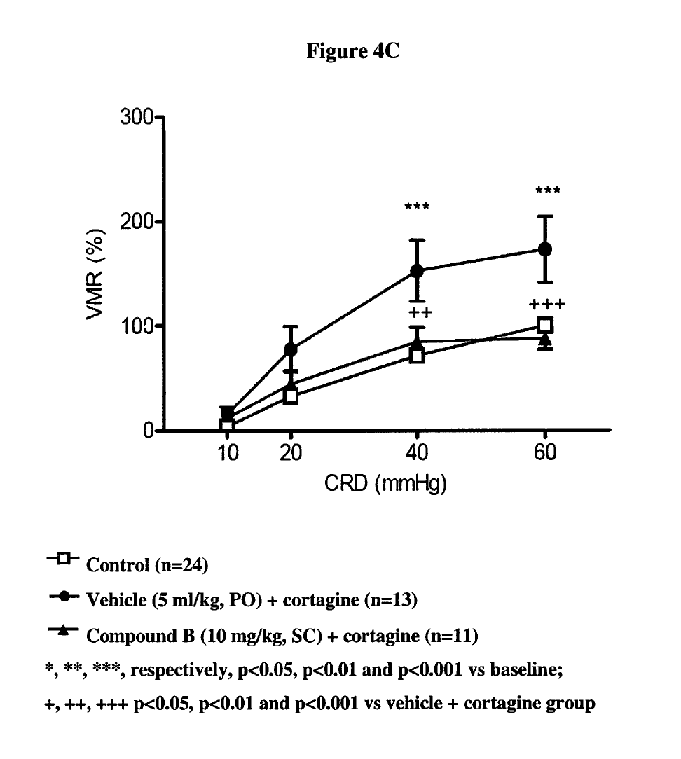

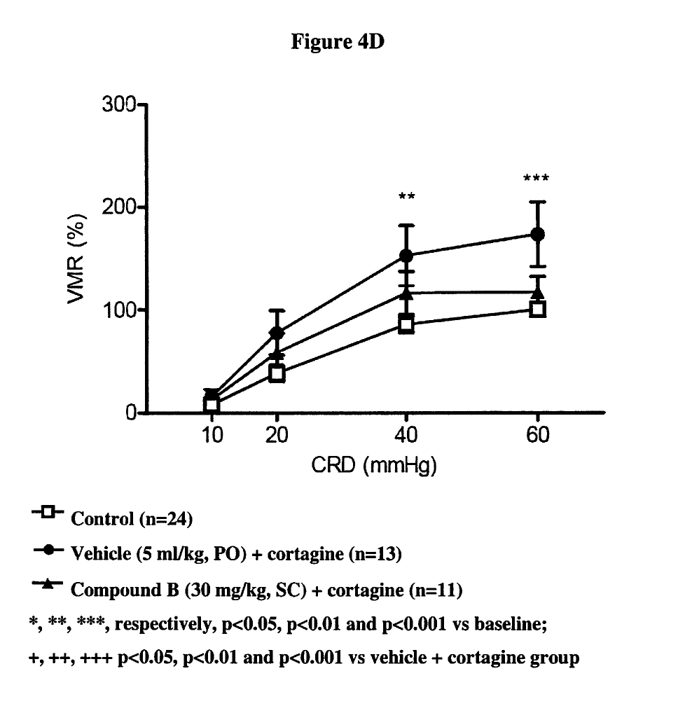

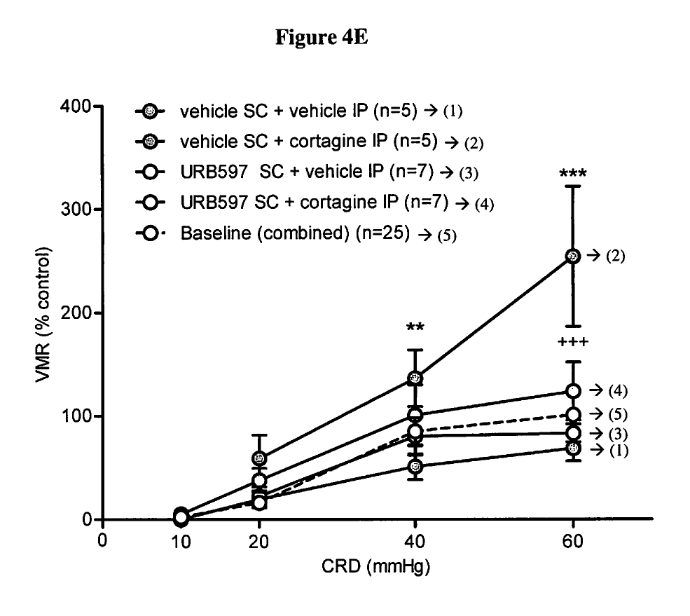

The visceromotor response (VMR) to baseline CRD in each of the five different groups of rats was similar at each pressure of distension of 10, 20 and 40 mmHg. At mmHg, data were 4.3±3.2, 4.3±4.0, 10.5±3.9, 2.9±2.0 and 10.8±3.9% for vehicle (n=13), compound A (n=11), URB597 (n=1), compound B 10 mg/kg (n=11) and compound B 30 mg/kg (n=1), respectively [F(4, 56)=1.164, p=0.3376]. At 20 mmHg, data were 42.2±10.9, 31.0±11.1, 33.4±8.4, 22.0±5.9 and 31.0±10.1% for vehicle, compound A, URB597, compound B 10 and 30 mg/kg, respectively [F(4, 56)-0.5893, p=0.6718]. At 40 mmHg, data were 87.5±7.5, 89.4±9.3, 89.4±14.6, 52.3±6.3 and 82.2±13.9% for vehicle, compound A, URB597, compound B 10 and 30 mg/kg, respectively [F(4, 56)=2.151, p=0.0875]. As baseline data were not significantly different between the different groups of rats, in each of the test conditions, (vehicle, compound A, URB597 and compound B 10 or mg/kg), the baseline of the sets of animals analyzed have been pooled together. In vehicle-treated rats (5 ml/kg, PO) (n=13), cortagine (10 μg/kg, IP) induced visceral hypersensitivity to colorectal distension. Compared to baseline, cortagine significantly increased the VMR to pressure distension of 40 and 60 mmHg (152.3±29.03 and 172.8±31.54 vs 87.45±7.55 and 100.0±0.0 for baseline, respectively, p<0.05) (FIGS. 4A-D). At 60 mmHg, 12/13 (92%) of rats presented an increase in VMR compared to baseline (i.e. visceral hypersensitivity in response to cortagine): 42% rats had increase between 0 and 50%, 33% increase between 51 and 100%, 17% increase over 101%.

The visceromotor response (VMR, y axis) was recorded for each colorectal distension pressure of 0 mmHg, 15 mmHg, 30 mmHg, 45 mmHg and 60 mmHg (x axis). FIG. 4A shows the results for baseline (n=24), represented by the symbols [symbol] ; the results for vehicle (5 ml/kg, PO)+cortagine (n=13), represented by the symbols [symbol] ; and the results for URB597 (3 mg/kg, SC)+cortagine (n=11), represented by the symbols [symbol] . URB597 (3 mg/kg, SC) (n=11) administered 2 h before cortagine injection abolished the visceral hypersensitivity in rats. URB597 blocked the increased VMR to CRD at 40 and 60 mmHg (81.89±16.38 and 97.62±20.02 vs 152.3±29.03 and 172.8±31.54 for cortagine, p<0.05 and p<0.01, respectively). At 60 mmHg, 7/11 (64%) of rats presented a decrease in VMR compared to baseline; 4/11 (36%) rats presented an increased VMR over baseline which ranged between 0 and 50% in 18% of rats, 51 and 100% in 9% of rats and over 101% in 9% of rats) (FIG. 4A). Baseline VMR (n=24) represents the pooled values of vehicle-treated rats (n=13) and URB597-treated rats (n=11).

FIG. 4B shows the results for baseline (n=24), represented by the symbols [symbol] ; the results for vehicle (5 ml/kg, PO)+cortagine (n=13), represented by the symbols [symbol] ; and the results for compound A (30 mg/kg, PO)+cortagine (n=11), represented by the symbols [symbol] . Compound A (30 mg/kg, PO) (n=1) administered 2 h before also showed a trend to decrease the cortagine-induced visceral hypersensitivity at both 40 and 60 mmHg and the number of rats that presented visceral hypersensitive responses at 60 mmHg (6/11, i.e. 55% rats), however the differences did not reach statistical significance (FIG. 4B). Out of these rats showing a reduced VMR at 60 mmHg, 18% exhibited increased VMR between 0 and 50%, 27% between 51 and 100% and 9 over 101% (FIG. 4B). Baseline VMR (n=24) represents the pooled values of baseline in vehicle-treated rats (n=13) and compound A-treated rats (n=11).

Compound B (10 mg/kg, PO) administered 2 h before cortagine injection abolished the visceral hypersensitivity in rats at all preures of distension. FIG. 4C shows the results for baseline (n=24), represented by the symbols [symbol] ; the results for vehicle (5 ml/kg, PO)+cortagine (n=13), represented by the symbols [symbol] ; and the results for compound B (10 mg/kg, PO)+cortagine (n=11), represented by the symbols [symbol] . FIG. 4D shows the results for baseline (n=24), represented by the symbols [symbol] ; the results for vehicle (5 ml/kg, PO)+cortagine (n=13), represented by the symbols [symbol] ; and the results for compound B (30 mg/kg, PO)+cortagine (n=11), represented by the symbols [symbol] . Data are mean±SEM. The symbols *, ** and *** represent values of p<0.05, p<0.01 and p<0.001, respectively, cf. baseline; the symbols +, ++ and +++ represent values of p<0.05, p<0.01 and p<0.001, respectively, cf. vehicle+cortagine group. ANOVA and multiple tests were used to assess the difference between treatment groups.

Compound B at 10 mg/kg PO decreased the increased VMR to CRD at 40 and 60 mmHg (84.74±13.54 and 87.75±10.96 vs 152.3±29.03 and 172.8±31.54 for cortagine, p<0.01 and p<0.001 respectively). At 60 mmHg, 7/11 (64%) of rats presented a decrease in VMR compared to baseline; 4/11 (36%) rats presented an increased VMR over baseline between 0 and 50% (FIG. 4C). Baseline VMR (n=24) represents the pooled values of vehicle-treated rats (n=13) and compound B (10 mg/kg)-treated rats (n=11).

Compound B (30 mg/kg, PO) administered 2 h before cortagine injection showed a trend to dampen the development of the visceral hypersensitivity in response to cortagine at both 40 mmHg and 60 mmHg, however the difference did not reach statistical significance. At 60 mmHg however, only 6/11 (55%) of rats presented an increase in VMR over baseline compared to the 92% of vehicle-treated rats with 45% (5/11) presenting an increased VMR between 0 and 50% and 9% increase over 101%) (FIG. 4D). Baseline VMR (n=24) represents the pooled values of vehicle-treated rats (n=13) and compound B (30 mg/kg)-treated rats (n=11).

### Second Trial of URB-597 Treatments

An additional experiment was performed on a separate rat population to test the effect of URB597 on cortagine-induced visceral hypersensitivity in rats, with the same protocol as given above except that an additional URB597+vehicle treatment was added to test any URB597 effect on vehicle-treated (i.e., non-hypersensitive) rats. Treatments and number of individuals in each treatment subpopulation were: (1) vehicle (sc)+vehicle (ip) (n=5); (2) vehicle (sc)+cortagine (ip) (n=5); (3) URB597 (sc)+vehicle (ip) (n=7), and (4) URB597 (sc)+cortagine (ip) (n=7). As above, on the day of the experiment, animals were equipped with distension balloons and placed in Bollman's cages before being brought to the experimental room, where they were left 20 min to recover from anesthesia. At the end of the min recovery period, a baseline CRD (CRD#1) of 40 min was performed at 10, 20, 40, 60 mmHg and the visceromotor response (VMR) assessed. Immediately after the end of the first CRD, rats received an oral gavage of vehicle (DMSO/cremophor/isotonic saline (1:1:8 v:v:v), 1.5 ml, n=10) or URB597 (3 mg/kg in vehicle, SC, n=14). Two hours after, half of the rats in each group received either injections of cortagine (10 μg/kg in vehicle, ip) or vehicle (ip). Fifteen minutes after these injections, a second CRD (CRD#2) of 40 min was performed. At the end of the distension, the balloons were removed prior to placing the rats back into their home cages (˜15 min).

### Conclusions

Cortagine injected IP induced visceral hypersensitivity to colorectal distension at 40 and 60 mmHg compared with baseline.

URB597 at 3 mg/kg injected SC as a 2 h pre-treatment prevented the visceral hypersensitivity induced by IP injection of cortagine at 40 and 60 mmHg (% hypersensitive rats: 55% versus 85% at 40 mmHg and 36% versus 92% at 60 mmHg).

In the second trial of URB597, Results of treatments (1)-(4) are shown on FIG. 4E along with the baseline CRD for all rats (n=25) for comparison. The trends were similar to the results shown in FIG. 4A. The VMR of vehicle+cortagine rats significantly differed from baseline at 40 mmHg CRD (p<0.01) and 60 mmHg CRD (p<0.001). Pretreatment of URB597 prior to administration of cortagine resulted in significant differences between VMR of the vehicle/cortagine rats at 60 mmHg CRD (p<0.001).

Compound A administered orally at 30 mg/kg as a 2 h pre-treatment showed a trend to reduce cortagine-induced visceral hypersensitivity at 60 mmHg which did not reach statistical significance (% hypersensitive rats: 55% versus 92% at 60 mmHg).

Compound B administered orally at 10 mg/kg as a 2 h pre-treatment prevented the visceral hypersensitivity by cortagine at 40 and 60 mmHg (% hypersensitive rats: 36% vs 85% at 40 mmHg and 36% versus 92% at 60 mmHg).

Compound B administered orally at 30 mg/kg as a 2 h pre-treatment shows a trend to reduce cortagine-induced visceral hypersensitivity at 60 mmHg which did not reach statistical significance (% hypersensitive rats: 55% versus 92% at 60 mmHg).

### Example 4

### Effect of a Single Administration of FAAH Inhibitor URB597 on Brain and GI Tissue Fatty Acid Amide Levels in Cortagine-Induced Visceral Hypersensitive Rats

Rats were treated with the FAAH inhibitor URB597 [3′-(aminocarbonyl) biphenyl-3-yl cyclohexylcarbamate](3 mg/kg, SC route) or vehicle (DMSO/cremophor/isotonic saline (1:1:8 v:v:v), PO route) in the cortagine-induced visceral hypersensitivity model as described in Example 3. Briefly Male Sprague-Dawley rats were habituated to Bollman cages for 3 days. The following day, rats were anesthetized with isoflurane and a 5 cm balloon pressure sensor was inserted into the distal colon 1 cm from the anus. After 20 min, a first colorectal distension (CRD) baseline was performed (10, 20, 40, 60 mmHg, 20 s duration, 4 min inter-stimulus) and the visceromotor response (VMR) assessed. After the first CRD, rats were treated with URB597 (3 mg/kg in vehicle, SC route) or vehicle (DMSO/cremophor/isotonic saline (1:1:8 v:v:v), PO route). Two hours later, rats were treated with cortagine (10 μg/kg in vehicle, IP route). After 15 min, a second CRD was performed.

Thirty min after the second CRD, rats were anesthetized with isoflurane, decapitated, and brains were collected rapidly, placed on sheets of aluminum foil sitting on dry ice to flash freeze. A 5 cm-long segment of jejunum and a 5 cm-long segment of ascending colon were collected, split open, dipped in ice-cold physiological saline to rinse away intestinal contents, placed on aluminum foil on top of dry ice to flash freeze. Then, brains, jejunum and ascending colon were placed in separate labeled Falcon tubes, respectively, and stored at −80° C.

On each experimental day, one naive untreated rat (n=6 total) (not treated with test compounds, not treated with cortagine and not subjected to CRD assay), kept in normal housing and handling conditions was also euthanized and its brain, jejunum and ascending colon collected as described above. The other rats were euthanized by CO₂ inhalation followed by thoracotomy.

Levels of the endocannabinoids N-arachidonoyl-ethanolamide (AEA, anandamide), N-oleoyl-ethanolamide (OEA) and N-palmitoyl-ethanolamide (PEA) in the brain, jejunum and ascending colon were measured by LC-MS/MS.

The fatty acid amides were extracted from the samples and standards by modifications of the method described in Richardson et al, Analytical Biochemistry, 2007, 360: 216-226, as described below.

### Rat Intestine Tissue Sample Preparation (for Colon and Jejunum Tissue Extraction)

Intestine samples were removed from the −80° C. freezer and placed on dry ice. Each intestine sample was weighed in a clean, tared 50 mL polypropylene BD Falcon tube and placed on wet ice after recording the weight. Using a glass pipette, 7 mL of (room temp.) 9:1 ethyl acetate:hexane were added to each intestine. Then, 2.5 μL of 5 μg/mL of IS (12.5 ng, prepared as indicated below) was added to each intestine sample. One conical container was removed at a time from the wet ice and homogenized until the tissue was uniformly minced. Using a homogenizer probe, the contents were homogenized for 30 seconds; 3 mL of water were added to each intestine sample, then homogenized for an additional 15 seconds. The homogenizer probe was rinsed with water between samples and with water and 70% EtOH between dose groups. The homogenates were vortexed for ˜5 sec then centrifuged at oC, 3500 rpm for 20 minutes. The ethyl acetate layer (top layer) was recovered and placed in 15 mL glass tubes. The solvent was evaporated under nitrogen (TurboVap, 55° C.) until dry and the tissue extracts were reconstituted in 0.25 mL of 1:3 CHCl₃:MeOH. Then the glass tubes were placed in a 37° C. shaking water bath for 5 min and vortexed again to re-suspend them. The samples were transferred to Eppendorf tubes and centrifuged (at room temp) at 13,000 rpm for 3 min. Then, 75 μL of each sample and standard was transferred to a 96 deep well plate on wet ice, and the remaining supernatant was stored at −80° C. The samples and standards were diluted to 1:1 using a multi-channel pipette by adding 75 μL of 100 ng/mL d-4-AEA in ice-cold methanol and pipetting up and down to mix. Diluted samples were placed in a chilled (6° C.) autosampler and analyzed by LC/MS/MS as described below. D4-AEA was purchased from Cayman Chemicals (catalog #10011178). A 5 μg/mL IS solution of N-palmitoyl propanolamide was prepared in methanol (using 1 mg/mL stock solution, stored −80° C.). 25 mL of a 100 ng/mL d-4-AEA solution in methanol were also prepared (using 1 mg/mL stock solution, stored −80° C.). Mixed solvent standards of 10×AEA, PEA and OEA at 10/100, 30/300, 100/1000, 300/3000, and 1000/10000 ng/mL were also prepared in methanol (from 1 mg/mL stock in DMSO, stored −80° C.). A 1×AEA, PEA and OEA mixed solvent standard curve was created by adding 50 μL of each 10×AEA, PEA or OEA standard to 450 μL of 1:3 CHCl₃:MeOH containing 50 ng/mL of N-palmitoyl propanolamide as internal standard (55 ng/mL IS diluent: 9.9 mL 1:3 CHCl₃:MeOH+110 μL of 5 μg/mL N-palmitoyl propanolamide IS as described above); final concentrations of 1×AEA, PEA and OEA standards in solvent were: 1/10, 3/30, 10/100, 30/300, and 100/1000 ng/mL.

### Brain Tissue Sample Preparation

Brain samples were removed from a −80° C. freezer and placed on dry ice. Individual brains were transferred to a clean, tared 50 mL capacity polypropylene conical tube and weights were recorded. A solution of ethyl acetate:hexanes (9:1) was immediately added to each conical tube along with internal standard (Palmitoyl Propanolamide). Samples were homogenized for 15 seconds using an electric-powered mechanical tissue disrupter (Omni Prep Multi-Sample Homogenizer Part Number: 06-021, Omni International, Kennesaw, Ga.) fitted with stainless steel probe (10 mm×110 mm Stainless Steel Omni Prep/THQ Homogenizer Probe, Omni International, Kennesaw, Ga.) washed with approximately 30% water and homogenized for 15 seconds more. Samples were vortexed and centrifuged at 1875×g for 30 minutes at 10° C. After centrifugation, the upper organic layer was recovered the samples were evaporated to dryness under nitrogen gas. Samples were not subjected to solid phase extraction. After reconstitution in 1 mL chloroform:methanol (1:3), samples were centrifuged (at 16000×g for 3 minutes at room temperature) to sediment any particulates. A 100 μL aliquot of each sample supernatant was transferred to individual wells on a 96 well plate. Each sample was diluted 1:1 with methanol containing internal standard (d-4-AEA) and analyzed by LC-MS/MS on a Waters Acquity/TQD system in positive ion (ES+) mode. Samples were maintained at 6° C. The final concentration of internal standards Palmitoyl Propanolamide and AEA-d4 in the samples was 50 ng/ml. Analyte quantification curves were generated using parallel sets of 4 unlabelled synthetic compounds serially diluted in a methanol diluent: AEA, PEA and OEA (each obtained from Cayman Chemical Inc, Ann Arbor Mich.). Two independent parallel standard curves were generated using analytes serially diluted using methanol as the diluent in the following manner: AEA (1-100 ng/mL), OEA (10-1000 ng/mL) and PEA (10-1000 ng/ml) in a single curve. The lower limit of quantitation was 1 ng/mL for AEA, 10 ng/mL for OEA and PEA. The samples were analyzed by LC-MS/MS on a Waters Acquity/TQD system in positive ion (ES+) mode. Samples were maintained at 6° C.

### Bioanalytical Assessment of AEA, OEA and PEA in Brain, Ascending Colon and Jejunum by LC-MS/MS

The concentrations of endogenous AEA, OEA and PEA levels were determined by LC-MS/MS using d-4-AEA, d-4-OEA and d-4-PEA stable isotope-labeled surrogate calibrators, with d8-AEA added as an internal standard (Cayman Chemicals, Ann Arbor, Mich.).

The samples were injected (10 μl) on a Clipeus C8 HPLC column (2.1 mm×30 mm dimensions; 5 μm particle size; with a Thermo BetaBasic 2.1×10 mm guard column; a column temperature of 40° C., a flow rate of 0.4 mL/min; a CTC PAL autosampler at 6° C.; Higgins Analytical, Mountain View, Calif.) and chromatographed under reverse phase conditions, using a gradient system with 5 mM ammonium acetate in water and 5 mM ammonium acetate in acetonitrile/isopropanol/water (80:15:5, v:v:v) and the gradient described in Table 1. The compounds were detected and quantified by tandem mass spectrometry in positive ion mode on an API4000 (Applied Biosystems; Framingham, Mass.). The limit of quantization for all three analytes was 0.3 ng/ml.

TABLE 1
Gradient protocol for the bioanalytical assessment of AEA, OEA
and PEA in brain, ascending colon and jejunum by LC-MS/MS.

| Time (min) | % A | % B |
|---|---|---|
| 0 | 70 | 30 |
| 0.5 | 70 | 30 |
| 2.5 | 5 | 95 |
| 4.5 | 5 | 95 |
| 5.0 | 70 | 30 |
| 7.0 | (end) | |

URB597 elevated levels of AEA, OEA and PEA in the brain, ascending colon and jejunum of cortagine-induced visceral hypersensitive rats.

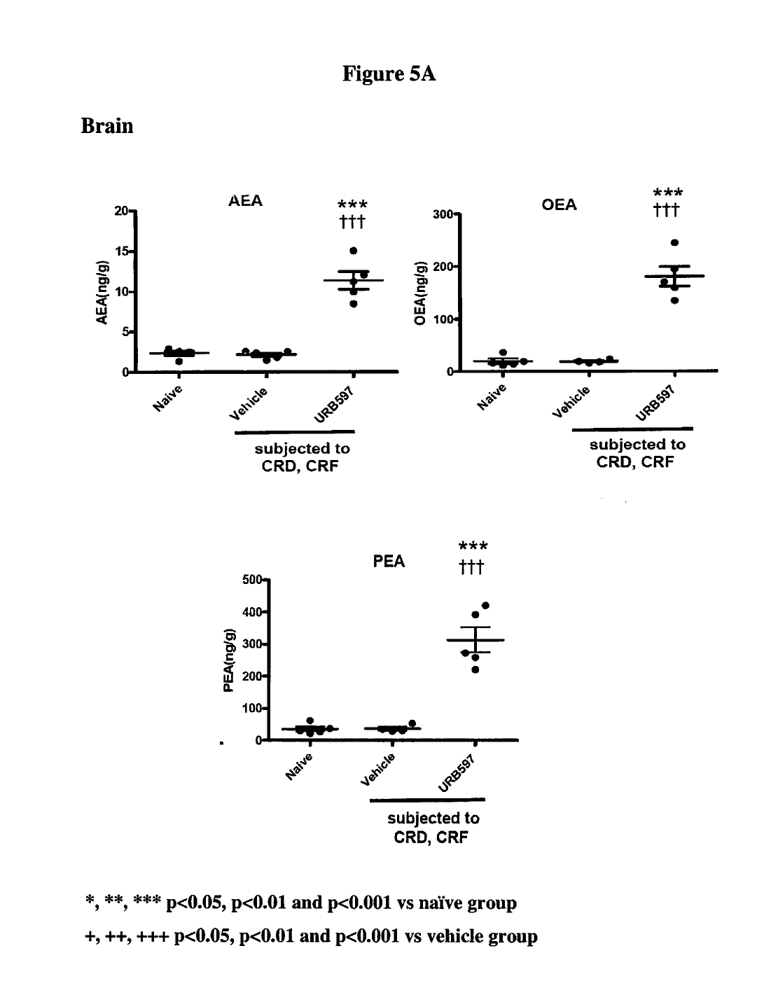

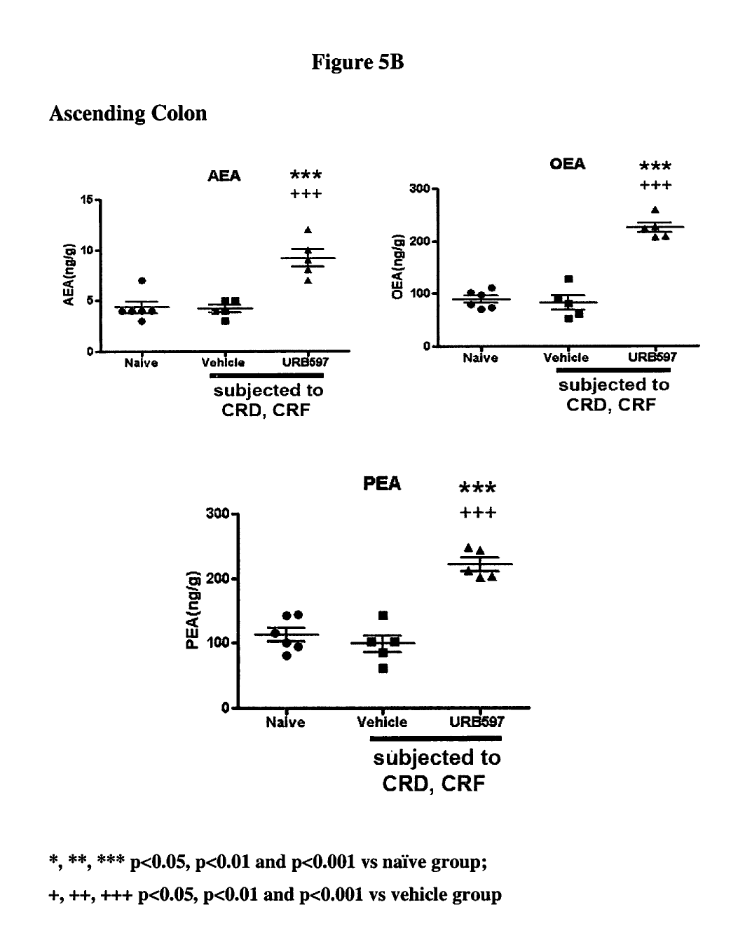

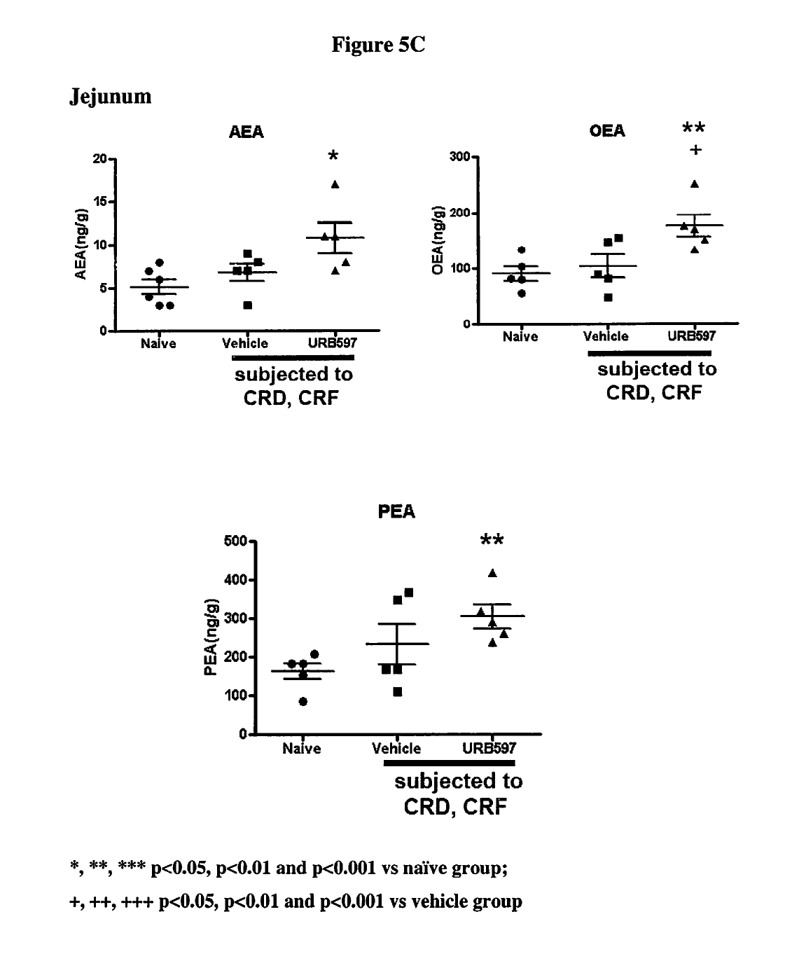

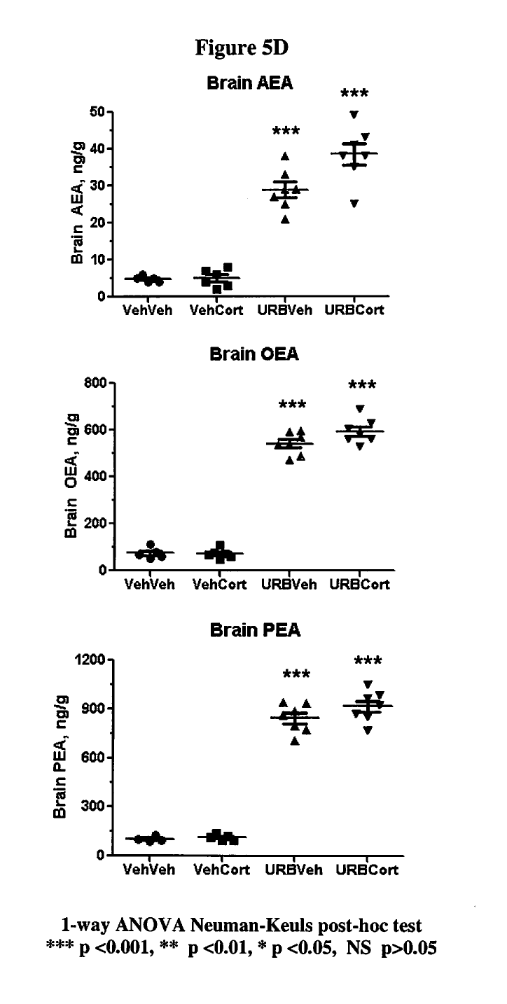

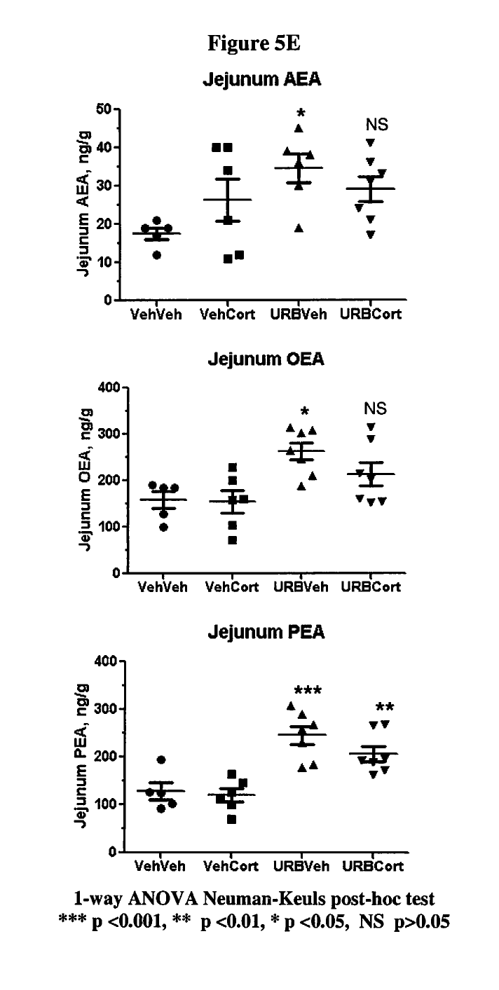

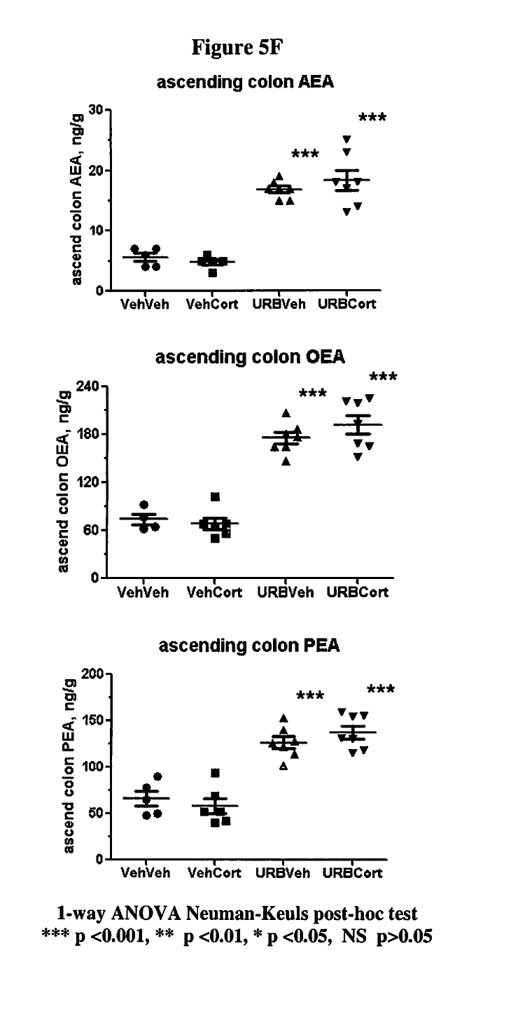

The FAAH inhibitor URB597 elevated levels of the three FAAH substrates (AEA, OEA, and PEA) in the brain, ascending colon and jejunum of cortagine-induced visceral hypersensitive rats (FIGS. 5A-C). AEA, OEA and PEA were quantified in brain, ascending colon and jejunum extracts by LC-MS/MS. FIG. 5A shows the AEA, OEA and PEA levels (y-axes, ng/g) in the brain of naïve, vehicle and URB597 treated rats. FIG. 5B shows the AEA, OEA and PEA levels (y-axes, ng/g) in the ascending colon of naïve, vehicle and URB597 treated rats. FIG. 5C shows the AEA, OEA and PEA levels (y-axes, ng/g) in the jejunum of naïve, vehicle and URB597 treated rats. The symbols *, ** and *** represent values of p<0.05, p<0.01 and p<0.001, respectively, vs. naïve group; the symbols +, ++ and +++ represent values of p<0.05, p<0.01 and p<0.001, respectively vs. vehicle group. Analysis by unpaired, two tailed Student's t test vs. respective vehicle or vs. naïve indicated that there was no statistical significance between vehicle and naïve rats for the levels of AEA, OEA, and PEA in the brain, jejunum and ascending colon. In the brain and ascending colon the levels of AEA, OEA, and PEA were statistically different from those in the vehicle treated and naïve rats (FIGS. 5A and 5B). In the jejunum, the levels of OEA were statistically different from those in the vehicle treated and naïve rats while the levels of AEA and PEA were statistically different from those in the naïve rats (FIG. 5C).

### Second Trial of URB597

An additional experiment was performed on a separate rat population to test the effect of URB597 and Cortagine on the three FAAH substrates (AEA, OEA, and PEA) in the brain, ascending colon and jejunum (FIGS. 5D-F) with the same protocol as given above except with the treatments were:vehicle (1:1:8 SC)+vehicle (saline IP); vehicle (1:1:8 SC)+Cortagine (10 ug/kg IP); URB597 (3 mg/kg SC)+vehicle (saline IP); URB597 (3 mg/kg SC)+Cortagine (10 ug/kg IP). FIG. 5D shows the AEA, OEA and PEA levels (y-axes, ng/g) in the brain of vehicle, cortagine, and URB597 treated rats. FIG. 5E shows the AEA, OEA and PEA levels (y-axes, ng/g) in the jejunum of vehicle, cortagine, and URB597 treated rats. FIG. 5F shows the AEA, OEA and PEA levels (y-axes, ng/g) in the ascending colon of vehicle, cortagine, and URB597 treated rats. Brain, jejunal, and ascending colon tissues were harvested from rats that underwent the second trial of URB597 discussed in Example 3. Harvesting protocols and assays for AEA, OEA, and PEA in each tissue were identical to those listed above.

Results for the brain, jejunum, and ascending colon tissue are shown in FIGS. 5D-5F. These results show that the FAAH inhibitor URB597 elevated levels of the three FAAH substrates (AEA, OEA, and PEA) in the brain, ascending colon, and jejunum of cortagine-induced visceral hypersensitive rats. The results also suggest that the direct administration of cortagine was not responsible for elevating the levels of the three FAAH substrates (AEA, OEA, and PEA) in the brain, ascending colon, and jejunum.

### Example 5

### Effect of the FAAH Inhibitor, URB 597, on Basal Sensitivity and the Partial Restraint Stress-Induced Hyperalgesia Model Experimental Protocols

The effect of FAAH inhibitor URB597 was tested at 3 mg/kg and 10 mg/kg in the PRS model of visceral hyperalgesia essentially as described in Examples 1 and 2. URB597 was formulated as a suspension in vehicle [DMSO/cremophor/isotonic saline (1:1:8 v:v:v)]. The concentration of URB597 was 1.5 mg/ml for the 3 mg/kg dose and 5 mg/ml for the 10 mg/kg dose.

### Treatments and Results

As described in Example 1, the effect of URB597 on basal sensitivity to colorectal distension was determined one day prior to conducting the partial restraint stress-induced hyperalgesia model. URB597 (3 mg/kg or 10 mg/kg) or vehicle was administered subcutaneously. The effects of the administered compounds on basal sensitivity to colorectal distension (CRD) were measured 3 hrs 15 minutes later. FIGS. 6A and 6B show the effects of either 3 mg/kg or 10 mg/kg URB597, respectively, in the basal sensitivity to colorectal distension model, indicating that URB597 does not alter basal sensitivity (normal responses to pain) in rats.

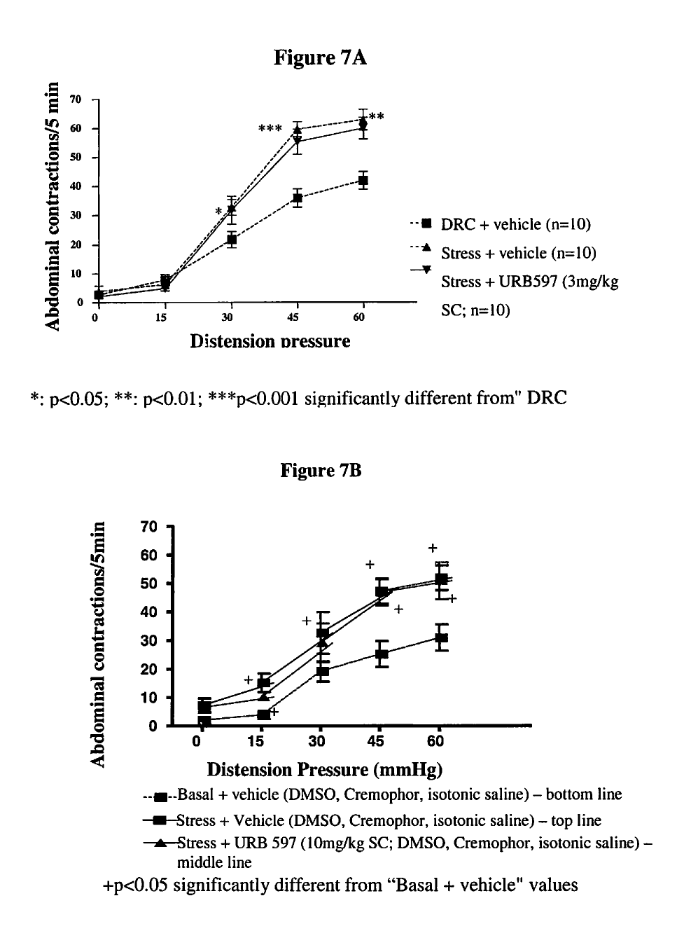

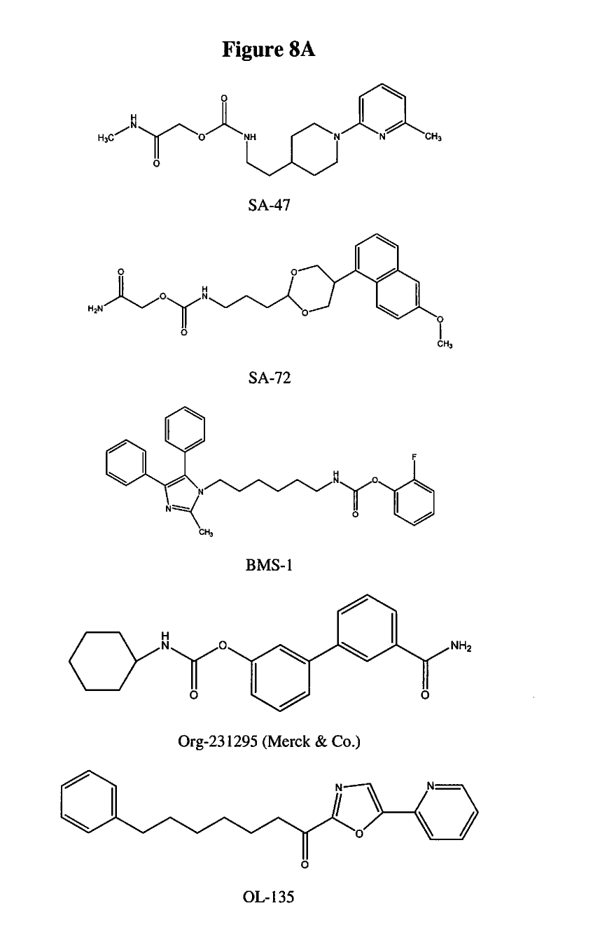

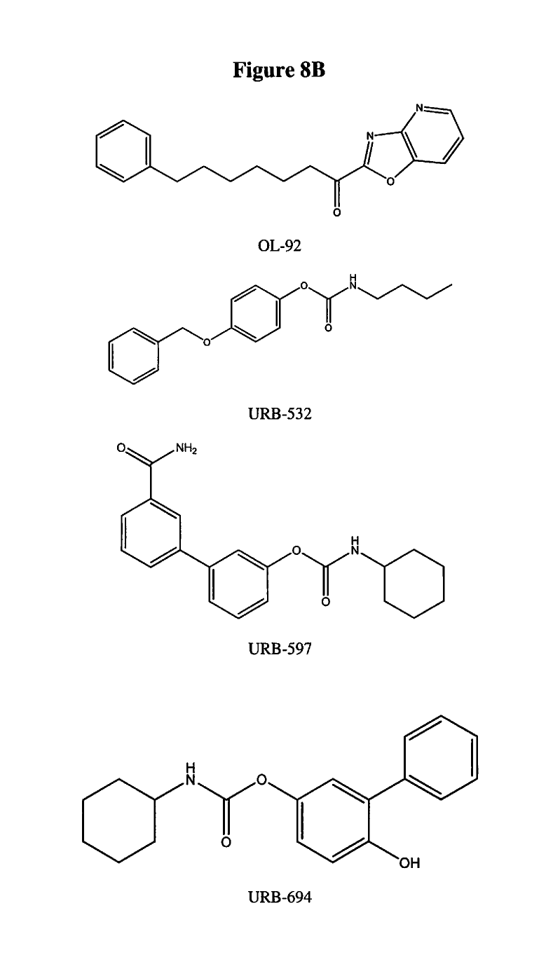

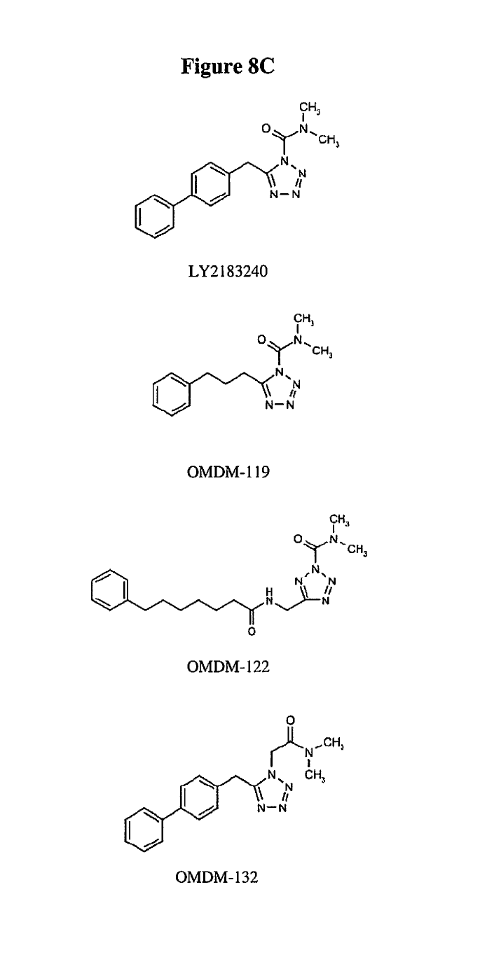

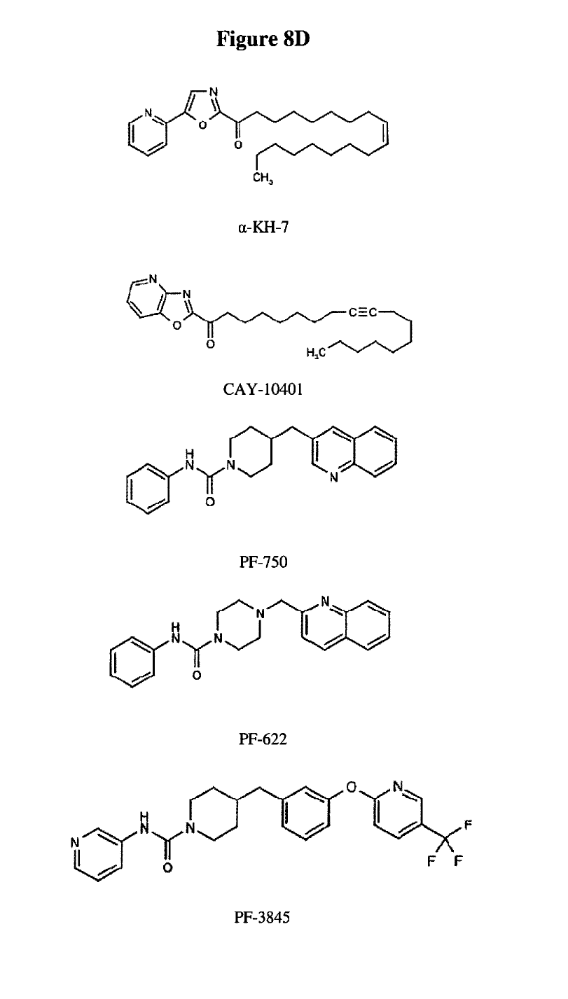

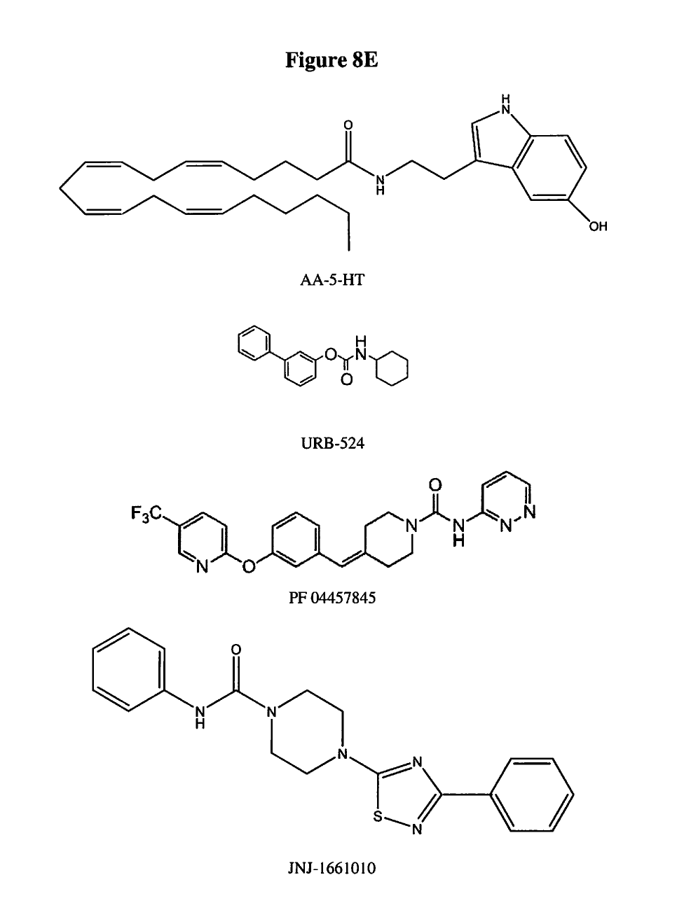

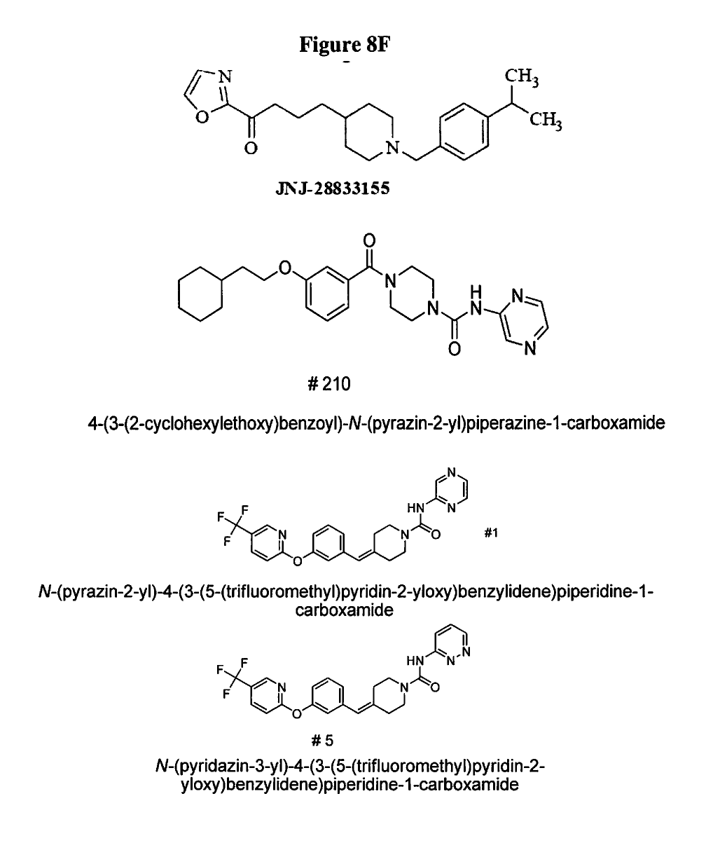

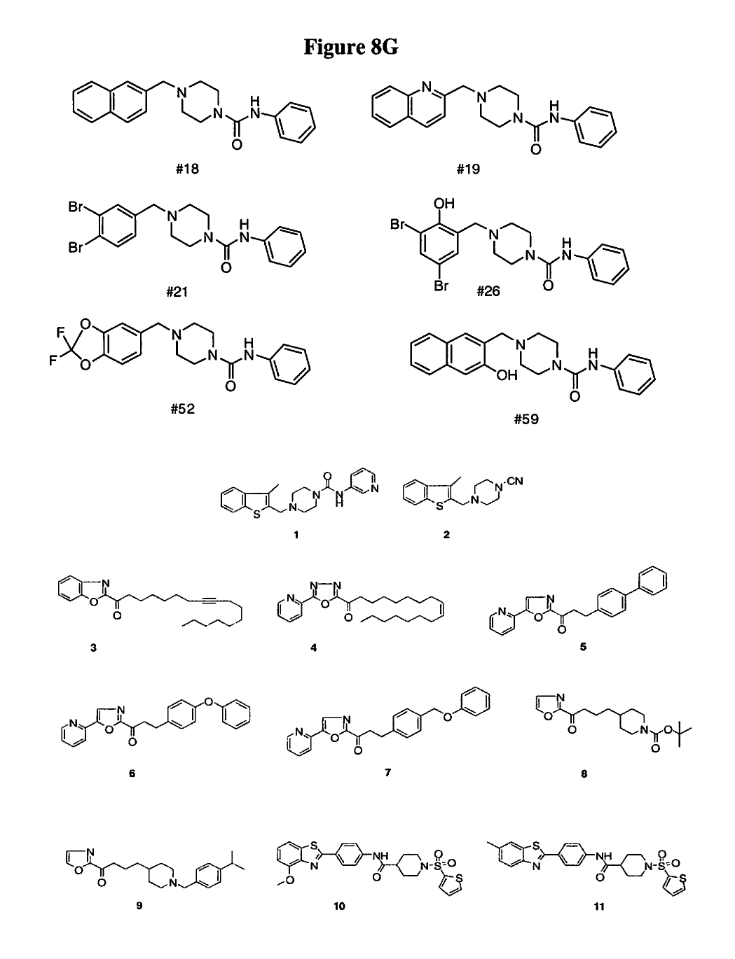

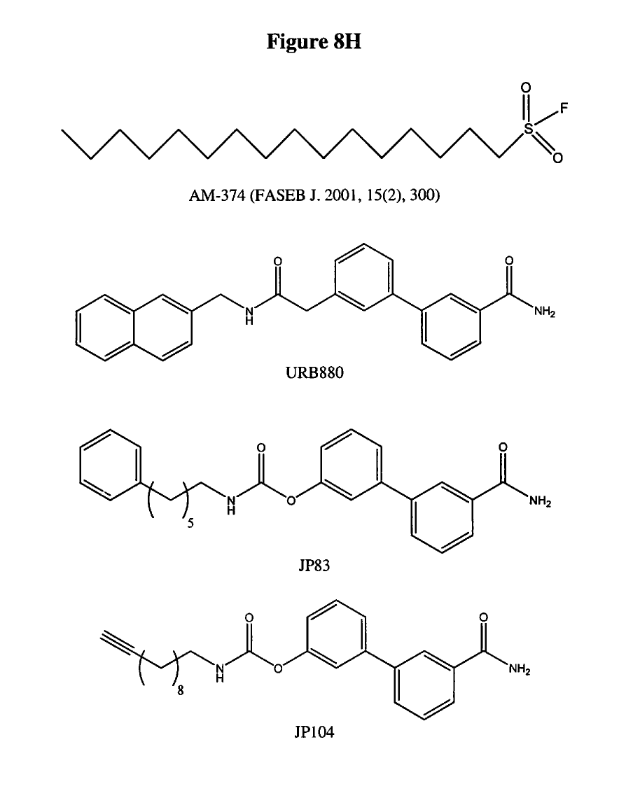

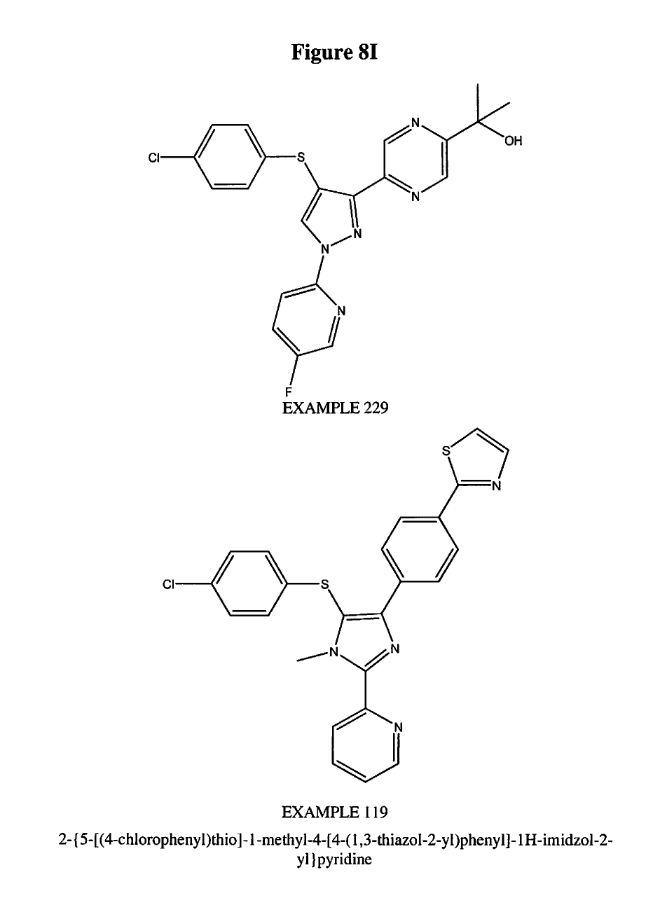

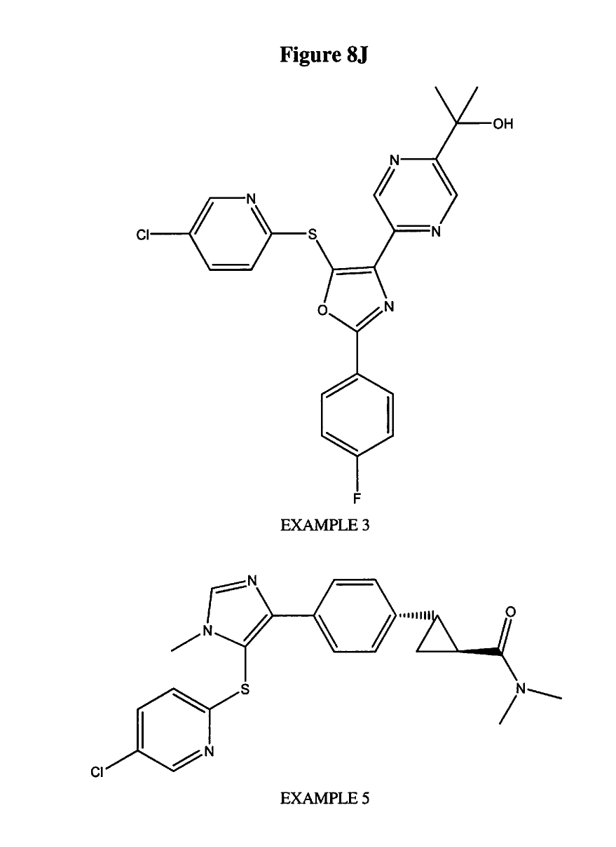

The following day, rats were administered either vehicle or URB597 one hour prior to PRS. Rats then received a two hour PRS session and CRD was conducted 15 minutes after the conclusion of the PRS. Under the tested conditions, no differences in number of abdominal contractions were observed for the vehicle or for URB-597 when tested at 3 mg/kg or 10 mg/kg (See FIGS. 7A and 7B).

### Conclusions

Although URB597 did not exhibit an antihyperalgesic effects in the PRS model, it did exhibit antihyperalgesic effects in the cortagine model of visceral pain (see Example 3). In addition, two other FAAH inhibitors, Compounds A and B, exhibit antihyperalgesic effects in both the PRS and cortagine models of visceral pain (see Examples 2 and 3). It is possible that no antihyperalgesic effect was observed in this experiment because the URB-597 formulation used did not deliver the expected dose. It is also possible that the pretreatment time with URB-597 was too long before CRD. (See Fegley et al. (2005), J. of Pharm. and Exp. Therapeutics 313:352-358).

Experiments are planned to evaluate fatty acid amide levels in brain and colon tissues of the animals in the PRS study of URB597 (3 mg/kg and 10 mg/kg SC) compared to vehicle (SC) to see if they were elevated when the CRD was performed.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

1-169. (canceled)

170. A method of treating or preventing abdominal, visceral or pelvic pain in a patient in need thereof, comprising administering a therapeutically or prophylactically effective amount of a FAAH inhibitor to said patient.

171. The method according to claim 170, wherein the pain is selected from:
(a) gastrointestinal pain: stomach pain, rectal pain, bowel pain, intestinal pain, intestinal cramps, pain and/or discomfort associated with irritable bowel syndrome (IBS), pain and/or discomfort associated with inflammatory bowel disease (IBD); pain and/or discomfort associated with functional dyspepsia, pain and/or discomfort associated with functional abdominal pain, pain and/or discomfort associated with ulcerative colitis, Crohn's disease or celiac disease; chest pain associated with gastro-esophageal reflux disease;
(b) pancreas pain, liver pain; cardiac pain;
(c) urological, renal or gynecological pain: kidney pain, ureter pain, bladder pain, prostate pain, gynecological pain, ovarian pain, uterine pain, labor pain, vulvar pain, vaginal pain, dysmenorrhea, dyspareunia, endometriosis, menstrual cramps, post-menopausal pelvic pain, pain and/or discomfort associated with vulvodynia, pain and/or discomfort associated with interstitial cystitis or painful bladder syndrome, pain and/or discomfort associated with prostatitis, pain associated with inflammatory pelvic disease.

172. The method according to claim 170, wherein the pain is abdominal, visceral or pelvic pain caused by cancer, by bacterial infections, viral infections, parasitic infections, surgery, trauma, exposure to noxious chemicals, medications, or digestive disorders; or wherein the pain is abdominal discomfort, soft-tissue pain, pain resulting from pancreatitis, gallbladder stones, kidney stones, diverticulitis, gastritis or is referred pain.

173. The method according to claim 170, wherein said FAAH inhibitor is a compound of Formula I or a pharmaceutically acceptable salt thereof: 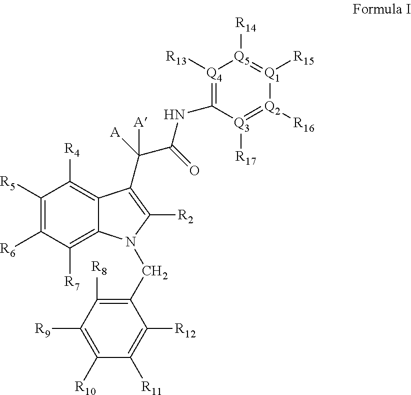
wherein:
each of Q₁, Q₂, Q₃, Q₄, and Q₅ are independently N or C;
A and A′ are independently: hydroxyl or an optionally independently substituted C₁ to C₃ alkoxy or A and A′ taken together are ═O, ═N(OH) or ═NOCH₃ or A and A′ together with the carbon to which they are attached form a cyclic ketal containing a total of 4 or 5 carbon atoms which can be optionally independently substituted;
R₂ is halogen, hydroxyl, —NO₂, an optionally independently substituted C₁-C₅ alkyl, an optionally independently substituted C₁-C₅ alkoxy, an optionally independently substituted C₂-C₅ alkenyl, an optionally independently substituted C₂-C₅ alkynyl, —CN, —C(O)OH, an optionally independently substituted cyclopropyl, —C(O)NR₂ₐR_{2b}, or —NR₂ₐR_{2b}, wherein R₂ₐ and R_{2b} are independently H or C₁-C₃ alkyl;
each of R₄, R₅, R₆ and R₇ is independently: H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C3-C6 cycloalkyl;
each of R₈, R₉, R₁₀, R₁₁ and R₁₂ is independently: H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
when Q₅ is C, R₁₄ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
when Q₅ is N, R₁₄ is missing;
when Q₂ is C, R₁₆ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, optionally independently substituted C₁-C₆ alkyl, or an optionally independently substituted C₃-C₆ cycloalkyl;
when Q₂ is N, R₁₆ is missing;
when Q₁ is C, R₁₅ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, optionally independently substituted C₁-C₆ alkyl, or an optionally independently substituted C₃-C₆ cycloalkyl;
when Q₁ is N, R₁₅ is missing;
when Q₄ is C, R₁₃ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, optionally independently substituted C₁-C₆ alkyl, or an optionally independently substituted C₃-C₆ cycloalkyl;
when Q₄ is N, R₁₃ is missing;
when Q₃ is C, R₁₇ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, optionally independently substituted C₁-C₆ alkyl, or an optionally independently substituted C₃-C₆ cycloalkyl;
and
when Q₃ is N, R₁₇ is missing.

174. The method of claim 173, wherein Q₁, Q₂, Q₃, Q₄, and Q₅ are C; R₂ is methyl; and
A and A′ taken together are ═O, then
(1) R₁₅ is not C(O)NH₂ and R₁₀ is not Cl;
(2) R₈, R₉, R₁₀, R₁₁, and R₁₂ are not all H and R₁₃ and R₁₇ are not both methyl; and
(3) R₈, R₉, R₁₀, R₁₁, R₁₂, R₁₃, R₁₄, R₁₅, R₁₆, R₁₇ are not all H; in said compounds of Formula I or pharmaceutically acceptable salts thereof.

175. The method according to claim 173, wherein said FAAH inhibitor is a compound of Formula A-2, Formula A-3 or Formula A-4 or a pharmaceutically acceptable salt thereof:

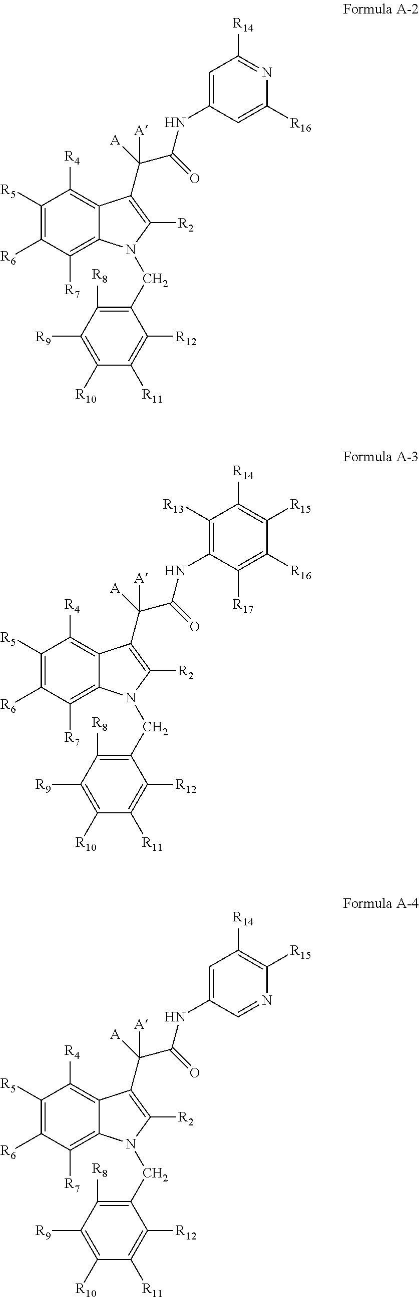

176. The method according to claim 173, wherein said FAAH inhibitor is a compound of Formula A-5 or Formula A-7 or a pharmaceutically acceptable salt thereof:

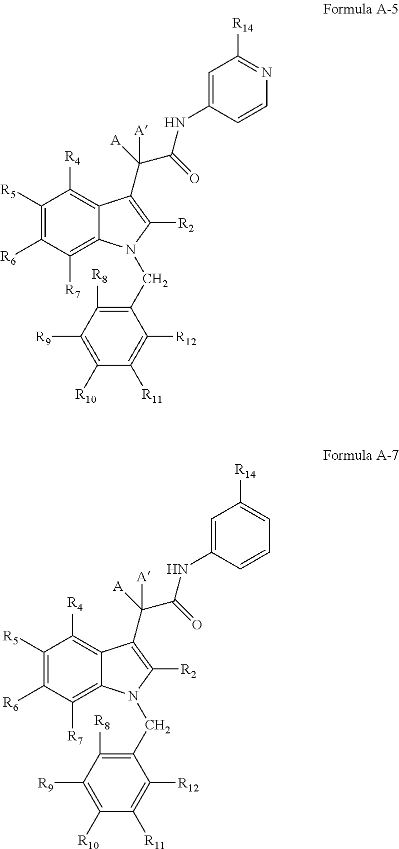

177. The method according to claim 173, wherein A and A′ taken together are ═O in said compound or pharmaceutically acceptable salt thereof.

178. The method according to claim 173, wherein R₂ is an optionally independently halogen substituted C₁-C₃ alkyl or cyclopropyl in said compound or pharmaceutically acceptable salt thereof.

179. The method according to claim 178, wherein R₂ is methyl in said compound or pharmaceutically acceptable salt thereof.

180. The method according to claim 173, wherein one or two of R₈, R₉, R₁₀, R₁₁ and R₁₂ are halogen and the rest are H in said compound or pharmaceutically acceptable salt thereof.

181. The method according to claim 180, wherein R₁₀ is Cl or F and R₈, R₉, R₁₁ and R₁₂ are H in said compound or pharmaceutically acceptable salt thereof.

182. The method according to claim 173, wherein R₄ and R₇ are H in said compound or pharmaceutically acceptable salt thereof.

183. The method according to claim 173, wherein R₆ is H in said compound or pharmaceutically acceptable salt thereof.

184. The method according to claim 173, wherein R₅ is selected from: ethoxy, methoxy, ethyl, methyl, halogen and H in said compound or pharmaceutically acceptable salt thereof.

185. The method according to claim 173, wherein each of R₁₃, R₁₅, R₁₆ and R₁₇ is independently selected from H, a halogen, —NO₂, —CN, —C(O)OH, hydroxyl, a C₁-C₅ alkyl, a C₂-C₅ alkenyl, a C₂-C₅ alkynyl, a C₁-C₅ alkoxy, —C(O)NRₐR_{b}, or —NRₐR_{b}, wherein Rₐ and R_{b} are independently H, a C₁-C₆ alkyl, or a C₃-C₆ cycloalkyl in said compound or pharmaceutically acceptable salt thereof.

186. The method according to claim 173, wherein R₁₄ is halogen or an optionally independently substituted methoxy and both R₁₃ and R₁₇ are H in said compound or pharmaceutically acceptable salt thereof.

187. The method according to claim 173, wherein said compound is selected from the following or a pharmaceutically acceptable salt thereof:
2-[1-(4-chlorobenzyl)-2-methyl-1H-indol-3-yl]-N-(2-chloropyridin-4-yl)-2-oxoacetamide
2-[1-(4-chlorobenzyl)-2-methyl-1H-indol-3-yl]-N-(3-methoxyphenyl)-2-oxoacetamide
2-[1-(4-chlorobenzyl)-5-methoxy-2-methyl-1H-indol-3-yl]-2-oxo-N-pyridin-2-ylacetamide
2-[2-chloro-1-(4-chlorobenzyl)-5-methoxy-1H-indol-3-yl]-2-oxo-N-pyridin-3-ylacetamide
2-[1-(4-chlorobenzyl)-5-methoxy-2-methyl-1H-indol-3-yl]-2-oxo-N-pyridin-4-ylacetamide
2-[1-(4-chlorobenzyl)-2,5-dimethyl-1H-indol-3-yl]-2-oxo-N-pyridin-4-ylacetamide
2-[1-(4-chlorobenzyl)-5-methoxy-2-methyl-1H-indol-3-yl]-2-oxo-N-phenylacetamide
2-[2-chloro-1-(4-chlorobenzyl)-5-methoxy-1H-indol-3-yl]-2-oxo-N-pyridin-4-ylacetamide
2-[2-chloro-1-(4-chlorobenzyl)-5-methoxy-1H-indol-3-yl]-2-oxo-N-pyrimidin-4-ylacetamide
2-[2-chloro-1-(4-chlorobenzyl)-5-methoxy-1H-indol-3-yl]-N-(2-chloropyridin-4-yl)-2-oxoacetamide
2-[1-(4-chlorobenzyl)-5-methoxy-2-methyl-1H-indol-3-yl]-N-(2-chloropyridin-4-yl)-2-oxoacetamide
2-[1-(4-chlorobenzyl)-5-ethoxy-2-methyl-1H-indol-3-yl]-2-oxo-N-phenylacetamide
2-[1-(4-chlorobenzyl)-5-methoxy-2-methyl-1H-indol-3-yl]-N-(3-methoxyphenyl)-2-oxoacetamide
2-[1-(4-chlorobenzyl)-2,5-dimethyl-1H-indol-3-yl]-2-oxo-N-phenylacetamide
2-[1-(2,4-dichlorobenzyl)-5-methoxy-2-methyl-1H-indol-3-yl]-2-oxo-N-pyridin-4-ylacetamide
2-[1-(4-chlorobenzyl)-5-methoxy-2-methyl-1H-indol-3-yl]-N-(3-chlorophenyl)-2-oxoacetamide
2-[1-(4-chlorobenzyl)-5-methoxy-2-methyl-1H-indol-3-yl]-2-oxo-N-pyrimidin-4-ylacetamide
2-[1-(4-chlorobenzyl)-2,5-dimethyl-1H-indol-3-yl]-2-oxo-N-pyridin-3-ylacetamide
2-[1-(4-chlorobenzyl)-5-methoxy-2-methyl-1H-indol-3-yl]-2-oxo-N-pyridin-3-ylacetamide
2-[1-(2,4-dichlorobenzyl)-5-methoxy-2-methyl-1H-indol-3-yl]-2-oxo-N-pyridin-3-ylacetamide
2-[1-(4-chlorobenzyl)-5-methoxy-2-methyl-1H-indol-3-yl]-N-(4-chlorophenyl)-2-oxoacetamide
2-[1-(4-chlorobenzyl)-5-methoxy-2-methyl-1H-indol-3-yl]-N-(4-methoxyphenyl)-2-oxoacetamide
2-[5-chloro-1-(4-chlorobenzyl)-2-methyl-1H-indol-3-yl]-2-oxo-N-pyridin-2-ylacetamide
2-[1-(4-chlorobenzyl)-2-isopropyl-5-methoxy-1H-indol-3-yl]-2-oxo-N-pyridin-4-ylacetamide
2-[1-(4-chlorobenzyl)-5-methoxy-2-methyl-1H-indol-3-yl]-N-(2-chlorophenyl)-2-oxoacetamide
2-[1-(4-chlorobenzyl)-2-isopropyl-5-methoxy-1H-indol-3-yl]-2-oxo-N-pyridin-3-ylacetamide
2-[1-(4-chlorobenzyl)-2-isopropyl-5-methoxy-1H-indol-3-yl]-2-oxo-N-phenylacetamide
2-[1-(4-chlorobenzyl)-5-methoxy-2-methyl-1H-indol-3-yl]-N-(2-methoxyphenyl)-2-oxoacetamide
2-[1-(4-chlorobenzyl)-5-ethoxy-2-methyl-1H-indol-3-yl]-2-oxo-N-pyridin-4-ylacetamide
2-[1-(4-chlorobenzyl)-2-methyl-1H-indol-3-yl]-2-oxo-N-pyridin-4-ylacetamide
2-[1-(4-chlorobenzyl)-5-hydroxy-2-methyl-1H-indol-3-yl]-2-oxo-N-phenylacetamide
2-[1-(4-chlorobenzyl)-2-methyl-1H-indol-3-yl]-2-oxo-N-pyridin-3-ylacetamide
2-[1-(4-chlorobenzyl)-2-methyl-1H-indol-3-yl]-2-oxo-N-phenylacetamide
N-(3-chlorophenyl)-2-[1-(2,4-dichlorobenzyl)-5-methoxy-2-methyl-1H-indol-3-yl]-2-oxoacetamide
2-[1-(2,4-dichlorobenzyl)-5-methoxy-2-methyl-1H-indol-3-yl]-N-(3-methoxyphenyl)-2-oxoacetamide
2-[1-(2,4-dichlorobenzyl)-5-methoxy-2-methyl-1H-indol-3-yl]-N-(5-methoxy-2-methylphenyl)-2-oxoacetamide
2-[1-(2,4-dichlorobenzyl)-5-methoxy-2-methyl-1H-indol-3-yl]-2-oxo-N-pyrimidin-4-ylacetamide
2-[1-(2,4-dichlorobenzyl)-5-methoxy-2-methyl-1H-indol-3-yl]-2-oxo-N-phenylacetamide
2-[1-(4-chlorobenzyl)-5-methoxy-2-methyl-1H-indol-3-yl]-N-(3-hydroxypyridin-2-yl)-2-oxoacetamide
2-[1-(4-chlorobenzyl)-2-methyl-1H-indol-3-yl]-2-oxo-N-pyrimidin-4-ylacetamide
2-[1-(4-chlorobenzyl)-2-methyl-1H-indol-3-yl]-N-(3-chlorophenyl)-2-oxoacetamide
2-[1-(4-chlorobenzyl)-5-hydroxy-2-methyl-1H-indol-3-yl]-2-oxo-N-pyridin-4-ylacetamide
2-[1-(4-chlorobenzyl)-5-methoxy-2-methyl-1H-indol-3-yl]-N-(2-fluorophenyl)-2-oxoacetamide
2-[1-(4-chlorobenzyl)-5-methoxy-2-methyl-1H-indol-3-yl]-N-(3,5-dichlorophenyl)-2-oxoacetamide
2-[1-(4-chlorobenzyl)-5-methoxy-2-methyl-1H-indol-3-yl]-N-(3-fluorophenyl)-2-oxoacetamide
2-[1-(4-chlorobenzyl)-5-methoxy-2-methyl-1H-indol-3-yl]-N-(4-fluorophenyl)-2-oxoacetamide
2-[1-(4-chlorobenzyl)-5-methoxy-2-methyl-1H-indol-3-yl]-N-(6-methoxypyrimidin-4-yl)-2-oxoacetamide
2-[2-chloro-1-(4-chlorobenzyl)-5-methoxy-1H-indol-3-yl]-N-(3-chlorophenyl)-2-oxoacetamide
2-[2-chloro-1-(4-chlorobenzyl)-5-methoxy-1H-indol-3-yl]-N-(3-methoxyphenyl)-2-oxoacetamide
2-[5-chloro-1-(4-chlorobenzyl)-2-methyl-1H-indol-3-yl]-2-oxo-N-pyridin-4-ylacetamide
2-[5-chloro-1-(4-chlorobenzyl)-2-methyl-1H-indol-3-yl]-2-oxo-N-pyrimidin-4-ylacetamide
2-[5-chloro-1-(4-chlorobenzyl)-2-methyl-1H-indol-3-yl]-N-(2-chloropyridin-4-yl)-2-oxoacetamide
2-[5-chloro-1-(4-chlorobenzyl)-2-methyl-1H-indol-3-yl]-N-(3-chlorophenyl)-2-oxoacetamide
2-[5-chloro-1-(4-chlorobenzyl)-2-methyl-1H-indol-3-yl]-N-(3-methoxyphenyl)-2-oxoacetamide
2-(1-benzyl-2,5-dimethyl-1H-indol-3-yl)-N-(2-methoxypyridin-4-yl)-2-oxoacetamide
2-(1-benzyl-2-methyl-1H-indol-3-yl)-N-(2-methoxypyridin-4-yl)-2-oxoacetamide
2-(1-benzyl-5-methoxy-2-methyl-1H-indol-3-yl)-N-(2-methoxypyridin-4-yl)-2-oxoacetamide
2-[1-(2,4-dichlorobenzyl)-2,5-dimethyl-1H-indol-3-yl]-N-(2-methoxypyridin-4-yl)-2-oxoacetamide
2-[1-(2,4-dichlorobenzyl)-2,5-dimethyl-1H-indol-3-yl]-N-(3-fluorophenyl)-2-oxoacetamide
2-[1-(2,4-dichlorobenzyl)-2-methyl-1H-indol-3-yl]-N-(2-methoxypyridin-4-yl)-2-oxoacetamide
2-[1-(2,4-dichlorobenzyl)-5-fluoro-2-methyl-1H-indol-3-yl]-N-(2-methoxypyridin-4-yl)-2-oxoacetamide
2-[1-(2,4-dichlorobenzyl)-5-methoxy-2-methyl-1H-indol-3-yl]-N-(2-methoxypyridin-4-yl)-2-oxoacetamide
2-[1-(2,4-difluorobenzyl)-2,5-dimethyl-1H-indol-3-yl]-N-(2-methoxypyridin-4-yl)-2-oxoacetamide
2-[1-(2,4-difluorobenzyl)-2-methyl-1H-indol-3-yl]-N-(2-methoxypyridin-4-yl)-2-oxoacetamide
2-[1-(2,4-difluorobenzyl)-5-methoxy-2-methyl-1H-indol-3-yl]-N-(2-methoxypyridin-4-yl)-2-oxoacetamide
2-[1-(2-chloro-4-fluorobenzyl)-2,5-dimethyl-1H-indol-3-yl]-N-(2-methoxypyridin-4-yl)-2-oxoacetamide
2-[1-(2-chloro-4-fluorobenzyl)-2-methyl-1H-indol-3-yl]-N-(2-methoxypyridin-4-yl)-2-oxoacetamide
2-[1-(2-chloro-4-fluorobenzyl)-5-methoxy-2-methyl-1H-indol-3-yl]-N-(2-methoxypyridin-4-yl)-2-oxoacetamide
2-[1-(2-chlorobenzyl)-5-methoxy-2-methyl-1H-indol-3-yl]-N-(2-methoxypyridin-4-yl)-2-oxoacetamide
2-[1-(3-chlorobenzyl)-5-methoxy-2-methyl-1H-indol-3-yl]-N-(2-methoxypyridin-4-yl)-2-oxoacetamide
2-[1-(4-chloro-2-fluorobenzyl)-2,5-dimethyl-1H-indol-3-yl]-N-(2-methoxypyridin-4-yl)-2-oxoacetamide
2-[1-(4-chloro-2-fluorobenzyl)-2-methyl-1H-indol-3-yl]-N-(2-methoxypyridin-4-yl)-2-oxoacetamide
2-[1-(4-chloro-2-fluorobenzyl)-5-methoxy-2-methyl-1H-indol-3-yl]-N-(2-methoxypyridin-4-yl)-2-oxoacetamide
2-[1-(4-chlorobenzyl)-2,5-dimethyl-1H-indol-3-yl]-2-oxo-N-pyrimidin-4-ylacetamide
2-[1-(4-chlorobenzyl)-2,5-dimethyl-1H-indol-3-yl]-N-(2-chloropyridin-4-yl)-2-oxoacetamide
2-[1-(4-chlorobenzyl)-2,5-dimethyl-1H-indol-3-yl]-N-(2-methoxypyridin-4-yl)-2-oxoacetamide
2-[1-(4-chlorobenzyl)-2,5-dimethyl-1H-indol-3-yl]-N-(3-chlorophenyl)-2-oxoacetamide
2-[1-(4-chlorobenzyl)-2,5-dimethyl-1H-indol-3-yl]-N-(3-methoxyphenyl)-2-oxoacetamide
2-[1-(4-chlorobenzyl)-2-methyl-1H-indol-3-yl]-N-(2-methoxypyridin-4-yl)-2-oxoacetamide
2-[1-(4-chlorobenzyl)-5-ethoxy-2-methyl-1H-indol-3-yl]-N-(2-chloropyridin-4-yl)-2-oxoacetamide
2-[1-(4-chlorobenzyl)-5-ethoxy-2-methyl-1H-indol-3-yl]-N-(2-methoxypyridin-4-yl)-2-oxoacetamide
2-[1-(4-chlorobenzyl)-5-ethoxy-2-methyl-1H-indol-3-yl]-N-(3-methoxyphenyl)-2-oxoacetamide
2-[1-(4-chlorobenzyl)-5-fluoro-2-methyl-1H-indol-3-yl]-2-oxo-N-pyridin-4-ylacetamide
2-[1-(4-chlorobenzyl)-5-fluoro-2-methyl-1H-indol-3-yl]-2-oxo-N-pyrimidin-4-ylacetamide
2-[1-(4-chlorobenzyl)-5-fluoro-2-methyl-1H-indol-3-yl]-N-(2-chloropyridin-4-yl)-2-oxoacetamide
2-[1-(4-chlorobenzyl)-5-fluoro-2-methyl-1H-indol-3-yl]-N-(2-methoxypyridin-4-yl)-2-oxoacetamide
2-[1-(4-chlorobenzyl)-5-fluoro-2-methyl-1H-indol-3-yl]-N-(3-chlorophenyl)-2-oxoacetamide
2-[1-(4-chlorobenzyl)-5-fluoro-2-methyl-1H-indol-3-yl]-N-(3-methoxyphenyl)-2-oxoacetamide
2-[1-(4-chlorobenzyl)-5-methoxy-2-methyl-1H-indol-3-yl]-2-oxo-N-[3-(trifluoromethoxy)phenyl]acetamide
2-[1-(4-chlorobenzyl)-5-methoxy-2-methyl-1H-indol-3-yl]-2-oxo-N-[3-(trifluoromethyl)phenyl]acetamide
2-[1-(4-chlorobenzyl)-5-methoxy-2-methyl-1H-indol-3-yl]-N-(2,6-difluorophenyl)-2-oxoacetamide
2-[1-(4-chlorobenzyl)-5-methoxy-2-methyl-1H-indol-3-yl]-N-(2-ethoxypyridin-4-yl)-2-oxoacetamide
2-[1-(4-chlorobenzyl)-5-methoxy-2-methyl-1H-indol-3-yl]-N-(2-fluoropyridin-4-yl)-2-oxoacetamide
2-[1-(4-chlorobenzyl)-5-methoxy-2-methyl-1H-indol-3-yl]-N-(2-methoxypyridin-4-yl)-2-oxoacetamide
2-[1-(4-chlorobenzyl)-5-methoxy-2-methyl-1H-indol-3-yl]-N-(3-chloro-4-fluorophenyl)-2-oxoacetamide
2-[1-(4-chlorobenzyl)-5-methoxy-2-methyl-1H-indol-3-yl]-N-(3-ethoxyphenyl)-2-oxoacetamide
2-[1-(4-chlorobenzyl)-5-methoxy-2-methyl-1H-indol-3-yl]-N-(3-ethylphenyl)-2-oxoacetamide
2-[1-(4-chlorobenzyl)-5-methoxy-2-methyl-1H-indol-3-yl]-N-(3-fluoropyridin-4-yl)-2-oxoacetamide
2-[1-(4-chlorobenzyl)-5-methoxy-2-methyl-1H-indol-3-yl]-N-(3-methylphenyl)-2-oxoacetamide
2-[1-(4-chlorobenzyl)-5-methoxy-2-methyl-1H-indol-3-yl]-N-(4-methoxypyridin-2-yl)-2-oxoacetamide
2-[1-(4-chlorobenzyl)-5-methoxy-2-methyl-1H-indol-3-yl]-N-(5-methoxypyridin-2-yl)-2-oxoacetamide
2-[1-(4-chlorobenzyl)-5-methoxy-2-methyl-1H-indol-3-yl]-N-(6-ethoxypyridin-3-yl)-2-oxoacetamide
2-[1-(4-chlorobenzyl)-5-methoxy-2-methyl-1H-indol-3-yl]-N-(6-methoxypyridin-2-yl)-2-oxoacetamide
2-[1-(4-chlorobenzyl)-5-methoxy-2-methyl-1H-indol-3-yl]-N-(6-methoxypyridin-3-yl)-2-oxoacetamide
2-[1-(4-fluorobenzyl)-2,5-dimethyl-1H-indol-3-yl]-N-(2-methoxypyridin-4-yl)-2-oxoacetamide
2-[1-(4-fluorobenzyl)-2-methyl-1H-indol-3-yl]-N-(2-methoxypyridin-4-yl)-2-oxoacetamide
2-[1-(4-fluorobenzyl)-5-methoxy-2-methyl-1H-indol-3-yl]-N-(2-methoxypyridin-4-yl)-2-oxoacetamide
2-[1-(4-fluorobenzyl)-5-methoxy-2-methyl-1H-indol-3-yl]-N-(3-fluorophenyl)-2-oxoacetamide
2-[2-chloro-1-(4-chlorobenzyl)-5-methoxy-1H-indol-3-yl]-N-(2-methoxypyridin-4-yl)-2-oxoacetamide
2-[2-chloro-1-(4-chlorobenzyl)-5-methyl-1H-indol-3-yl]-2-oxo-N-pyridin-4-ylacetamide
2-[2-chloro-1-(4-chlorobenzyl)-5-methyl-1H-indol-3-yl]-2-oxo-N-pyrimidin-4-ylacetamide
2-[2-chloro-1-(4-chlorobenzyl)-5-methyl-1H-indol-3-yl]-N-(2-chloropyridin-4-yl)-2-oxoacetamide
2-[2-chloro-1-(4-chlorobenzyl)-5-methyl-1H-indol-3-yl]-N-(2-methoxypyridin-4-yl)-2-oxoacetamide
2-[2-chloro-1-(4-chlorobenzyl)-5-methyl-1H-indol-3-yl]-N-(3-chlorophenyl)-2-oxoacetamide
2-[2-chloro-1-(4-chlorobenzyl)-5-methyl-1H-indol-3-yl]-N-(3-fluorophenyl)-2-oxoacetamide
2-[2-chloro-1-(4-chlorobenzyl)-5-methyl-1H-indol-3-yl]-N-(3-methoxyphenyl)-2-oxoacetamide
2-[2-chloro-1-(4-fluorobenzyl)-5-methoxy-1H-indol-3-yl]-2-oxo-N-pyridin-4-ylacetamide
2-[2-chloro-1-(4-fluorobenzyl)-5-methoxy-1H-indol-3-yl]-2-oxo-N-pyrimidin-4-ylacetamide
2-[2-chloro-1-(4-fluorobenzyl)-5-methoxy-1H-indol-3-yl]-N-(2-chloropyridin-4-yl)-2-oxoacetamide
2-[2-chloro-1-(4-fluorobenzyl)-5-methoxy-1H-indol-3-yl]-N-(2-methoxypyridin-4-yl)-2-oxoacetamide
2-[2-chloro-1-(4-fluorobenzyl)-5-methoxy-1H-indol-3-yl]-N-(3-chlorophenyl)-2-oxoacetamide
2-[2-chloro-1-(4-fluorobenzyl)-5-methoxy-1H-indol-3-yl]-N-(3-fluorophenyl)-2-oxoacetamide
2-[2-chloro-1-(4-fluorobenzyl)-5-methoxy-1H-indol-3-yl]-N-(3-methoxyphenyl)-2-oxoacetamide
2-[5-chloro-1-(4-chlorobenzyl)-2-methyl-1H-indol-3-yl]-N-(2-methoxypyridin-4-yl)-2-oxoacetamide
2-[5-chloro-1-(4-fluorobenzyl)-2-methyl-1H-indol-3-yl]-N-(2-methoxypyridin-4-yl)-2-oxoacetamide
2-[5-fluoro-1-(4-fluorobenzyl)-2-methyl-1H-indol-3-yl]-N-(2-methoxypyridin-4-yl)-2-oxoacetamide
2-[5-methoxy-1-(4-methoxybenzyl)-2-methyl-1H-indol-3-yl]-N-(2-methoxypyridin-4-yl)-2-oxoacetamide
2-[5-methoxy-2-methyl-1-(4-methylbenzyl)-1H-indol-3-yl]-N-(2-methoxypyridin-4-yl)-2-oxoacetamide
2-{5-methoxy-2-methyl-1-[4-(trifluoromethoxy)benzyl]-1H-indol-3-yl}-N-(2-methoxypyridin-4-yl)-2-oxoacetamide
2-{5-methoxy-2-methyl-1-[4-(trifluoromethyl)benzyl]-1H-indol-3-yl}-N-(2-methoxypyridin-4-yl)-2-oxoacetamide
N-(2-chloropyridin-4-yl)-2-[1-(2,4-dichlorobenzyl)-5-methoxy-2-methyl-1H-indol-3-yl]-2-oxoacetamide
N-(2-chloropyridin-4-yl)-2-[1-(4-fluorobenzyl)-5-methoxy-2-methyl-1H-indol-3-yl]-2-oxoacetamide
N-(2-chloropyridin-4-yl)-2-[5-methoxy-1-(4-methoxybenzyl)-2-methyl-1H-indol-3-yl]-2-oxoacetamide
N-(3-chlorophenyl)-2-[1-(4-fluorobenzyl)-5-methoxy-2-methyl-1H-indol-3-yl]-2-oxoacetamide
N-(3-chlorophenyl)-2-[5-methoxy-1-(4-methoxybenzyl)-2-methyl-1H-indol-3-yl]-2-oxoacetamide
N-(3-fluorophenyl)-2-[5-methoxy-1-(4-methoxybenzyl)-2-methyl-1H-indol-3-yl]-2-oxoacetamide.

188. The method according to claim 170, wherein said FAAH inhibitor is administered before or after a symptom of pain develops in said patient.

189. A method of treating or preventing pain in a patient in need thereof, comprising administering a therapeutically or prophylactically effective amount of a FAAH inhibitor to said patient in combination with a therapeutically or prophylactically effective amount of one or more additional therapeutic agents.

190. The method according to claim 189, wherein the additional therapeutic agent is selected from: a painkiller, a Mu opioid receptor agonist, a non-steroidal anti-inflammatory drug (NSAID), a pain relieving agent, an opiate receptor agonists, a cannabinoid receptor agonist, an anti-infective agent, a sodium channel blocker, a N-type calcium channel blocker, a local anesthetic, a VR1 agonist, an anti-inflammatory and/or immunosuppressive agent, an antidepressant, an anti-emetic agent, a corticosteroid, a proton pump inhibitor, a leukotriene antagonist, a nicotinic acetylcholine receptor agonist, a P2X3 receptor antagonist, a NGF agonist and antagonist, a NK1 and NK2 antagonist, a NMDA antagonist, a GABA modulator, an anti-cancer agent, an anti-hyperlipidemia drug, an appetite suppressing agent, an anti-diabetic medication, a serotonergic and noradrenergic modulator, a GI agent, a GCC (Guanylate Cyclase C) agonist, a 5HT4 agonist, a 5HT3 antagonist, a bile acid sequestrant, a mast cell stabilizer, an anti-diarrheal compound, or a combination of two or more of the above thereof.

191. The method according to claim 190, wherein said FAAH inhibitor is administered prior to, simultaneously, or after the initiation of treatment by an additional therapeutic agent.

192. The method according to claim 190, wherein
(a) said painkiller is acetaminophen or paracetamol;
(b) said Mu opioid receptor agonist is loperamide;
(c) said non-steroidal anti-inflammatory drug is selected from: propionic acid derivatives (e.g., alminoprofen, benoxaprofen, bucloxic acid, carprofen, fenhufen, fenoprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (meclofenamic acid, mefe-namic acid, and tolfenamic acid), biphenyl-carboxylic acid derivatives, oxicams (isoxicam, meloxicam, piroxicam, sudoxicam and tenoxican), salicylates (acetyl salicylic acid, sulfasalazine), pyrazolones (apazone, bezpiperylon, feprazone, mofebutazone, oxyphenbutazone, phenylbutazone), or a COX-2 inhibitor, such as, for example, a COX-2 inhibitor in the coxibs family (celecoxib, deracoxib, valdecoxib, rofecoxib, parecoxib, nimesulide, etoricoxib);
(d) said other pain relieving agent is gabapentin, topical capsaicin, tanezumab, esreboxetine or pregabalin;
(e) said opiate receptor agonist is morphine, propoxyphene (Darvon™), tramadol, hydrocodone, oxycodoneor buprenorphin;
(f) said cannabinoid receptor agonist is Dronabinol™, Δ9-THC, CP-55940, WIN-55212-2, HU-210, cannabis, marijuana, marijuana extract, levonatradol, Sativex™, nabilone, ajulemic acid or Cannador®
(g) said sodium channel blocker is carbamazepine, mexiletine, lamotrigine, lidocaine, tectin, NW-1029 or CGX-1002;
(h) said N-type calcium channel blocker is ziconotide, NMED-160, SPI-860; serotonergic and noradrenergic modulators such as SR-57746, paroxetine, duloxetine, clonidine, amitriptyline or citalopram; or an anticonvulsant such as gabapentin and pregabalin.
(i) said VR1 agonist and antagonist is NGX-4010, WL-1002, ALGRX-4975, WL-10001 or AMG-517;
(j) said anti-inflammatory and/or immunosuppressive agent is methotrexate, cyclosporin A (including, for example, cyclosporin microemulsion), tacrolimus, corticosteroids, statins, interferon beta, Remicade (Infliximab™), Enbrel (Etanercept™) or Humira (Adalimumab™);
(k) said antidepressant is an SSRIs (e.g., fluoxetine, citalopram, femoxetine, fluvoxamine, paroxetine, indalpine, sertraline, zimeldine), a combined SSRI and 5HT1A partial agonist (e.g., vilazodone), a tricyclic antidepressant (e.g., imipramine, amitriptiline, chlomipramine and nortriptiline), a therapeutic antidepressant (e.g., bupropion and amineptine) or an SNRIs (e.g., venlafaxine and reboxetine);
(l) said 5HT3 antagonist is ondansetron (Zofran™), granisetronmetoclopramide, ramosetron (Irribow™) or alosetron (Lotronex™);
(m) said corticosteroid is betamethasone, budesonide, cortisone, dexamethasone, hydrocortisone, methylprednisolone, prednisolone, prednisone or triamcinolone;
(n) said proton pump inhibitor is omeprazole, lansoprazole, rabeprazole, esomeprazole or pantroprazole.
(o) said leukotriene antagonist is zafirlukast, montelukast, pranlukast.
(oo) said 5-lipoxygenase inhibitors is zileuton or PF-04191834;
(p) said nicotinic acetylcholine receptor agonist is ABT-202, A-366833, ABT-594; BTG-102, A-85380 or CGX1204;
(q) said P2X3 receptor antagonist is A-317491, ISIS-13920 or AZD-9056;
(r) said NGF agonist and antagonist is tazenumab, RI-724, RI-1024, AMG-819, AMG-403 or PPH 207;
(s) said NK1 and NK2 antagonist is DA-5018, R-116301; CP-728663 or ZD-2249;
(t) said NMDA antagonist is NER-MD-11, CNS-5161, EAA-090, AZ-756, CNP-3381; potassium channel modulators is CL-888, ICA-69673 or retigabine;
(u) said GABA modulator is lacosamide or propofol;
(v) said anti-cancer agent is tyrosine kinase inhibitors imatinib (Gleevec/Glivec™) or gefitinib (Iressa™), fluorouracil, 5-FU (Adrucil™), bevacizumab (Avastin™), irinotecan (Camptosar™), oxaliplatin (Eloxatin™), cetuximab (Erbitux™), panitumumab (Vectibix™), leucovorin (Wellcovorin™) or capecitabine (Xeloda™);
(w) said anti hyperlipidemia drug is a statin, ezetimibe or niacin;
(x) said appetite suppressing agent is sibutramine, taranabant or rimonabant;
(y) said anti-diabetic medication is insulin, tolbutamide (Orinase™), acetohexamide (Dymelor™), tolazamide (Tolinase™), chlorpropamide (Diabinese™), glipizide (Glucotrol™), glyburide (Diabeta™, Micronase™, Glynase™), glimepiride (Amaryl™), gliclazide (Diamicron™), repaglinide (Prandin™), nateglinide (Starlix™), pramlintide (Symlin™) or exanatide (Byetla™);
(z) said serotonergic or noradrenergic modulator is SR-57746, paroxetine, duloxetine, clonidine, amitriptyline, citalopram, or flibanserin;
(aa) said GI agent is a laxative (e.g. lubiprostone (Amitiza™), Fybogel®, Regulan®, Normacol® and the like), a gastrointestinal agent used for the treatment of idiopathic chronic constipation and constipation-predominant IBS, a GI motility stimulant (e.g. domperidone, metoclopramide, mosapride, itopride) or an antispasmodic drug (e.g. anticholinergics, hyoscyamine or dicyclomine);
(bb) said GCC (Guanylate Cyclase C) agonists is linaclotide;
(cc) said 5HT4 agonist is tegasarod;
(dd) said bile acid sequestrant is questran, cholesevelan, sevelamer, cholestipol or cholestyramine;
(ee) said mast cell stabilizer is cromolyn or nedocromil; and
(ff) said anti-diarrhea compound is octreotide, an antiperistaltic agent (e.g. loperamide (Imodium™, Pepto Diarrhea™)), tamoxifen, a bulking agent, an anti-estrogen (e.g. droloxifene, TAT-59 orraloxifene), tormentil root extract (Potejntilla tormentilla) from the family Rosaceae, bismuth subsalicylate (e.g. Pepto-Bismol™), diphenoxylate, diphenoxylate with atropine (Lomotil™, Lomocot™), oat bran, psyllium, calcium carbonate or an astringent (e.g., tannins).
